(19) United States
(12) Patent Application Publication
(10) Pub. No.: US 2011/0099650 A1
(43) Pub. Date: Apr. 28, 2011

(54) COMPOSITIONS AND METHOD FOR MODULATING PLANT ROOT HAIR DEVELOPMENT

(75) Inventors: Liam Dolan, Norwich (GB); Benoit Menand, Marseille (FR); Keke Yi, Norwich (GB)

(73) Assignee: PLANT BIOSCIENCE LIMITED, Norwich Norfolk (GB)

(21) Appl. No.: 12/451,574

(22) PCT Filed: Apr. 9, 2008
(86) PCT No.: PCT/GB2008/001246

§ 371 Date: Nov. 19, 2009

(30) Foreign Application Priority Data
May 22, 2007 (GB) 0709835.3

(51) Int. Cl. A01H 5/00; A01H 1/06; C07H 21/04

(52) U.S. Cl. 800/260; 800/278; 800/298; 536/23.6

(57) ABSTRACT

This invention relates to the modulation of root hair development in plants by altering the expression of RHD6-related genes, for example to increase the number, length and/or longevity of root hairs in the plant. This may be useful, for example, in improving the ability of plants to extract nutrients from the soil.

### FIELD OF INVENTION

This invention relates to the modulation of root hair development in plants.

### BACKGROUND OF THE INVENTION

In 1990, Schiefelbein and Somerville” published a paper describing their work with Arabidopsis thaliana mutants in their efforts to understand genetic control of root hair development. They examined roots from 12,000 mutagenized Arabidopsis seedlings, leading to identification of more than 40 mutants impaired in root hair morphogenesis. Mutants were characterized as belonging to four phenotypic classes which genetically were produced from single nuclear recessive mutations in four different genes designated RHD1, RHDP, RHD3, and RHD4. As a result of the phenotypic analysis of the mutants and homozygous double mutants, a model for root hair development was proposed, including the stages at which the genes are normally required. The RHD1 gene product appears to be necessary for proper initiation of root hairs, whereas the RHDS, RHD3, and RHD4 gene products are required for normal hair elongation. These authors concluded that the results they obtained demonstrate that root hair development in Arabidopsis is amenable to genetic dissection and should prove to be a useful model system to study the molecular mechanisms governing cell differentiation in plants.

In 1994, Masucci and Schiefelbein⁷ extended those results by identifying another mutant, the rhd6 mutant, concluding that root-hair initiation in Arabidopsis thaliana provides a model for studying cell polarity and its role in plant morphogenesis. They observed that root hairs normally emerge at the apical end of root epidermal cells, implying that these cells are polarized. The rhd6 mutant was characterized as displaying three defects: (a) a reduction in the number of root hairs, (b) an overall basal shift in the site of root hair emergence, and (c) a relatively high frequency of epidermal cells with multiple root hairs. They concluded that these defects implicate the RHD6 gene in root-hair initiation and indicate that RHD6 is normally associated with the establishment of, or response to, root epidermal cell polarity. Similar alterations in the site of root-hair emergence, although less extreme, were also discovered in roots of the auxin-, ethylene-, abscisic acid-resistant mutant axr2 and the ethylene-resistant mutant etrl. All three rhd6 mutant phenotypes were rescued when either auxin (indoleacetic acid) or an ethylene precursor (1-aminocyclopropane-1-carboxylic acid) was included in the growth medium. The rhd6 root phenotypes could be phenocopied by treating wild-type seedlings with an inhibitor of the ethylene pathway (aminoethoxyvinylglycine). These results indicate that RHD6 is normally involved in directing the selection or assembly of the root-hair initiation site through a process involving auxin and ethylene.

Root hairs play important roles in plant nutrition and water uptake. In most soils they are important for phosphate and iron uptake. In drought conditions they are important in the uptake of other nutrients such as nitrate. Therefore the manipulation of root hair traits will be important in developing crops that can effectively extract nutrients from the soil. Until now this has been difficult since no gene with a function limited to the root hair has been identified.

EP0803572B1 discloses the identification, isolation, cloning, and characterization of the CPC gene of Arabidopsis thaliana, for regulating initiation of root hair formation, as well as transgenic plants over-expressing the CPC gene. The CPC gene is not responsible for the rhd mutant phenotypes described above. This is confirmed, for example, in U.S. Pat. No. 661,749, as well as EP0803572B1 itself.

### SUMMARY OF INVENTION

The present invention relates to the finding that the over-expression of ROOT HAIR DEFECTIVE 6 (RHD6) genes in plants alters root hair development, for example leading to plants with an increased number, length and/or longevity of root hairs. Furthermore, over-expression of a different gene family (ROOT HAIR DEFECTIVE SIX LIKE1 (RSL) genes) produces a similar effect. Modulation of the expression of these genes (collectively termed ‘RHD6-related genes’) in plants may be useful, for example, in manipulating root hair traits in diverse groups of plant species (including crops) to improve their ability to extract nutrients from the soil.

An aspect of the invention provides an isolated ROOT HAIR DEFECTIVE 6 (RHD6)-related gene.

RHD6-related genes include both ROOT HAIR DEFECTIVE 6 (RHD6) genes and ROOT HAIR DEFECTIVE SIX LIKE1 (RSL1) genes, and functional homologues thereof, as described herein. RHD6-related genes include genes capable of complementing the rhd6 mutation in plants.

Another aspect of the invention provides an isolated gene encoding an amino acid sequence encoded by the RHD6-related gene or a gene product that is sufficiently homologous thereto to permit, on production thereof in an rhd6 mutant cell a functional complementation of said mutation.

Another aspect of the invention provides an isolated product of the expression of an isolated RHD6-related gene.

Another aspect of the invention provides an isolated polynucleotide which encodes a gene product comprising an amino acid sequence shown in SEQ ID NO: 1, 3, 5, 7, 9, 11, 13 to 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112 and 114.

Another aspect of the invention provides an isolated polynucleotide which has at least 40% nucleic acid sequence identity with one or more of SEQ ID NOS: 2, 4, 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115.

Other aspects of the invention provide expression constructs, plant cells, and plants or plant progeny, including seeds, which comprise an isolated RHD6-related gene or polynucleotide described herein.

Another aspect of the invention provides a method of modulating root hair development in a plant comprising;
increasing the expression of an RHD6-related polypeptide within cells of said plant relative to control plants.

An RHD6-related polypeptide may, for example, comprise an amino acid sequence shown in SEQ ID NO: 1, 3, 5, 7, 9, 11, 13 to 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112 or 114.

Another aspect of the invention provides a method of improving the tolerance of a plant to nutrient-deficient conditions comprising;
increasing the expression of an RHD6-related polypeptide within cells of said plant relative to control plants.

Another aspect of the invention provides a method of increasing the production of a root-secreted phytochemical in a plant comprising;
increasing the expression of an RHD6-related polypeptide within cells of a plant which produces the root-secreted phytochemical.

In some embodiments, expression of an RHD6-related polypeptide may be increased in a plant by expressing a heterologous nucleic acid encoding said RHD6-related polypeptide within cells of said plant.

In some embodiments, expression of an RHD6-related polypeptide may be increased in a plant by;
crossing a first and a second plant to produce a population of progeny plants;
determining the expression of the RHD6-related polypeptide in the progeny plants in the population, and
identifying a progeny plant in the population in which expression of the RHD6-related polypeptide is increased relative to controls.

In some embodiments, expression of an RHD6-related polypeptide may be increased in a plant by;
exposing a population of plants to a mutagen,
determining the expression of the RHD6-related polypeptide in one or more plants in said population, and;
identifying a plant with increased expression of the RHD6-related polypeptide.

Plants identified as having increased expression of the RHD6-related polypeptide may be sexually or asexually propagated or grown to produce off-spring or descendants showing increased expression of the RHD6-related polypeptide.

Another aspect of the invention provides a method of producing a plant with altered root-hair development comprising:
incorporating a heterologous nucleic acid which alters the expression of a RHD6-related polypeptide into a plant cell by means of transformation, and;
regenerating the plant from one or more transformed cells.

Another aspect of the invention provides a plant produced by a method described herein which displays altered root-hair development relative to controls.

### BRIEF DESCRIPTION OF THE DRAWINGS

FIG. 1 shows that AtRHD6 is a positive regulator of root hairs development in Arabidopsis. FIG. 1a shows roots of Atrhd6-1, Atrhd6-2 and Atrhd6-3 mutants with their respective wild type and complementation of the Atrhd6-3 mutant with a genomic AtRHD6p::GFP:AtRHD6 fusion. FIG. 1b shows a fluorescent image of the genomic AtRHD6p::GFP:AtRHD6 fusion in the Atrhd6-3 background showing AtRHD6 protein in hair cells nuclei. FIG. 1c shows the expression of the Atrhd6-2 enhancer trap GUS gene in root cross section. FIG. 1d shows a whole mount longitudinal view of the expression of the enhancer trap GUS gene in Atrhd6-2 and in different backgrounds (cpc, wer, ttg1 and gl2). H, hair cell; N, non hair cells; C, cortex. Scales bars, 500 μm (a), 50 μm (b), 25 μm (c) and 100 μm (d).

FIG. 2 shows that AtRSL1 positively regulates root hairs development in Arabidopsis. FIG. 2a shows roots of WT, Atrhd6-3 single mutant, Atrsl1-1 single mutant, Atrhd6-3 Atrsl1-1 double mutant and Atrhd6-3 Atrsl1-1 double mutant bearing the AtRSL1p::GFP:AtRSL1 transgene. Plants were grown on MS media with sucrose overlaid with a cellophane disc to increase root hairs production in the Atrhd6-3 mutant. FIG. 2b shows a fluorescent image of the genomic AtRSL1p::GFP:AtRSL1 fusion in the Atrhd6-3 Atrsl1-1 background showing AtRSL1 protein in hair cells nuclei. H, hair cell; N, non hair cells. Scale bars, 500 μm (a) and 50 μm (b).

FIG. 3 shows the relationship between RHD6-LIKE proteins from Arabidopsis and Physcomitrella. The tree is a strict consensus tree of 12 most parsimonious tree generated using the alignment of bHLH domains amino acids sequences shown in Tables 1 and 2. The Arabidopsis genes used are the members of bHLH subfamily VIIIc, except AtIND (INDEHISCENT)/At4g00120 which was used as out-group and belongs to the bHLH subfamily VIIIb^{8, 10, 26}. Physcomitrella PpRSL1 to 7 sequences were obtained by BLAST of the Physcomitrella genomic sequence. PpIND1 is a Physcomitrella sequence similar to AtIND and a putative member of family VIIIb in Physcomitrella. Numbers are bootstrap values and indicates an 82% level of confidence for the occurrence of the AtRHD6 clade. The brackets indicates the AtRHD6 clade and the sister clade.

FIG. 4 shows that PpRSL1 and PpRSL2 positively control the development of caulonemal cells and rhizoids in Physcomitrella and PpRSL1 and AtRHD6 have a conserved molecular function. FIGS. 4a and b show eighteen day old protonema from WT, Pprsl1 and Pprsl2 single mutants, and Pprsl1 Pprsl2 double mutant, grown from spores on 0.8% agar. FIG. 4a shows whole protonema growing from a single spore. FIG. 4b shows dissected filaments from protonema shown in FIG. 4a. FIG. 4c shows isolated one month old gametophores. FIG. 4d shows roots of the Arabidopsis Atrhd6-3 mutant carrying the 35S::PpRSL1 transgene compared to WT and Atrhd6-3 roots. ca, caulonemal cell; ch, chloronemal cell; rh, rhizoid. Scale bars, 1 mm (a), 100 μm (b), 1 mm (c), and 500 μm (d).

FIG. 5 shows the phenotype for the transformants:35S::RHD6 FIG. 5A shows col-0 rhd6/rsl1 with 35S::RHD6; FIG. 5B shows col-0 rhd6/rsl1 with 35S::RHD6; FIG. 5C. rhd6/rsl1 with 35S::RHD6

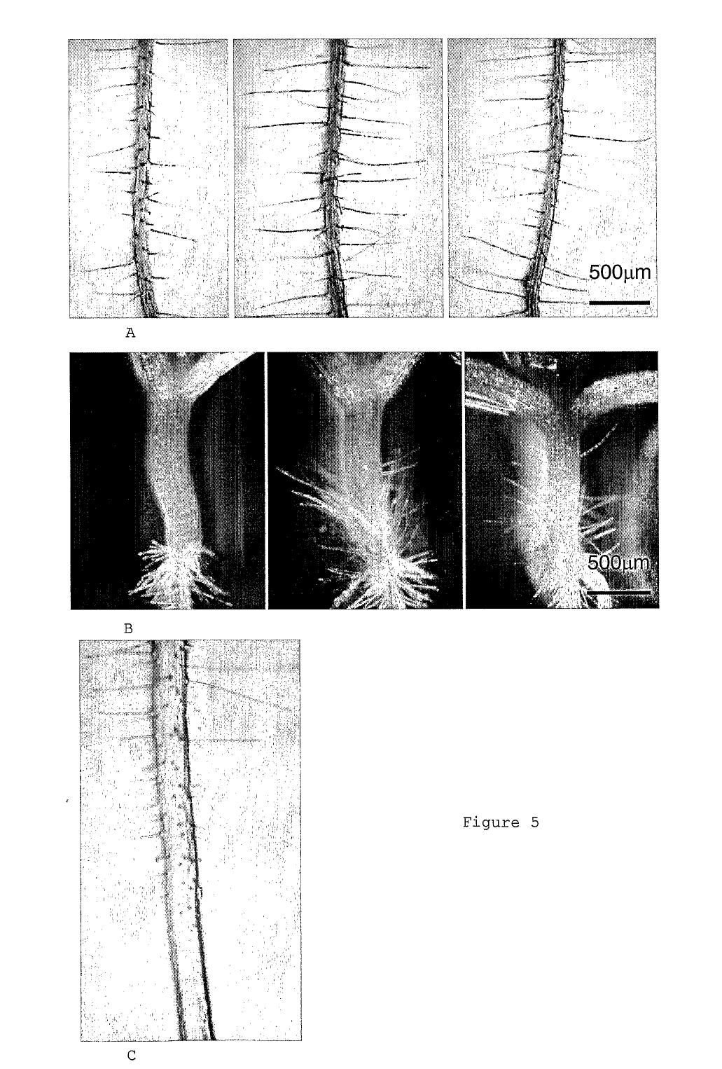

FIG. 6 shows the phenotype for the transformants:35S::RSL2 and 35S::RSL3 FIG. 6A shows col-0 rhd6/rsl1 with 35S::RSL2/3; FIG. 6B shows root hypocotyls

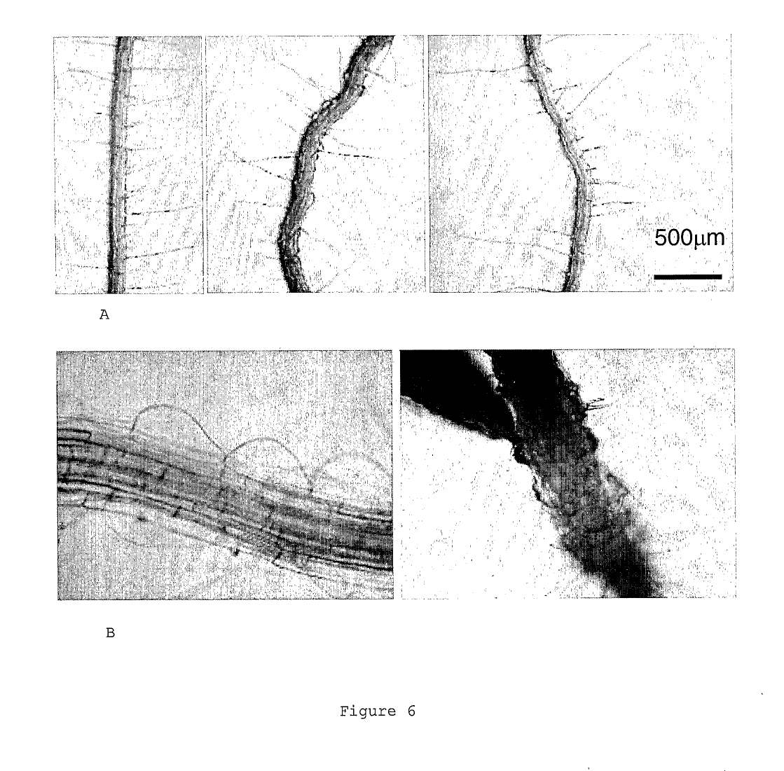

FIG. 7 shows the molecular basis of mutations in A. thaliana AtRHD6 (A) and AtRSL1 (B) genes. White boxes correspond to coding regions (black boxes for the bHLH domain encoding region). Grey triangles indicate the position of each insertion. Numbers in brackets indicate the distance between each T-DNA insertion and the start codon. (C) RT-PCR showing that Atrhd6-3, Atrsl1-1 and Atrhd6-3 Atrsl1-1 are RNA null mutants. AtAPT1, Adenine phosphoribosyltransferase 1.

FIG. 8 shows that the Atrhd6-3 and Atrsl1-1 single mutants and the Atrhd6 Atrsl1 double mutant have no detectable pollen tube growth defects. Ratios of resistance to antibiotic of F2 plants from Atrhd6-3 Atrsl1-1 double mutant backcrossed to WT are 76.7% (n=1404; X²(3/1)=2.19; P>0.05) for Sulfadiazin (resistance carried by the Atrhd6-3 allele) and 74.9% (n=1289; X²(3/1)=0.0023; P>0.05) for phosphinothricin (resistance carried by the Atrsl1-1 allele) showing normal segregation of the single mutants and double mutant gametes. (FIG. 8A shows pollen of the genotype indicated below each picture was used to pollinate WT stigma. Carpels were stained with aniline blue 4 hours after pollination. The growth of each mutant pollen tubes in the WT carpel is revealed by callose staining in blue (white arrows). Similar pollen tube growth is observed in WT and mutant pollen tubes. FIG. 9B shows in vitro pollen tube growth experiment. WT and mutants pollens were germinated on agar plates. Representative plates are shown with the germination ratio (mean of 600 pollen grains per line, with standard error). Similar germination ratios are observed between WT and mutants pollen (Student's-t-test p values are 0.554 for Atrhd6-3 versus WT, 0.904 for Atrsl1-1 versus WT and 0.87 for Atrhd6-3 Atrsl1-1 versus WT). Scale bars, 200 μm (FIGS. 8A and B).

FIG. 9 shows the molecular basis of P. patens Pprsl1, Pprsl2 and Pprsl1 Pprsl2 mutations (three independent mutants, named 1 to 3, are shown in each case). FIGS. 9A and D show the structure of the PpRSL1 (A) and PpRSL2 (D) genes (up), and the expected result of the homologous recombination (down). White boxes correspond to coding regions (black boxes for the bHLH domain encoding region) and the grey boxes correspond to the resistance gene cassette (NptII and AphIV). The regions of homology used for gene replacement are delimited by grey lines. The distance between the restrictions sites used for Southern blots and the position of the probes used are also shown. FIGS. 9B, C, E and F show southern blots of WT and mutants DNA digested with ScaI (B and C) or NcoI (E and F) and hybridized with the probe indicated below the picture. Blots C and F are hybridization of the same membrane used for blot B and E respectively, after stripping of the gene specific probe. The replacement of the WT band by a larger band of expected size (see A and D) in mutants lines when hybridization is performed with the gene specific probe (B and E), and the hybridization of only the mutant band with the resistance gene probe (C and F), demonstrate the presence of single insertions in the PpRSL1 and PpRSL2 loci. (G) RT-PCR showing that the mutants are RNA null mutants. PpGAPDH, glyceraldehyde 3-phosphate dehydrogenase. In each case, the three independent single insertion mutants presented have the same phenotype and only the mutant 1 is shown in FIG. 4.

FIG. 10 shows the root hair system of an Arabidopsis plant over-expressing RSL4 and displaying a root morphology resembling a fungal symbiont, such as Mycorrhizae.

### DETAILED DESCRIPTION OF THE PREFERRED EMBODIMENTS OF THE INVENTION

The present disclosure demonstrates the identification, isolation, cloning and expression of the ROOT HAIR DEFECTIVE 6 (RHD6) and ROOT HAIR DEFECTIVE SIX LIKE (RSL) genes (collectively termed ‘RHD6-related genes’ herein) in plants. It shows complementation of mutations by distantly related genes, providing the function of root hair development in plants in which the distantly related gene has been inactivated. Accordingly, those skilled in the art will appreciate that, for the first time, the gene responsible for previously identified mutant phenotypes has been isolated and cloned according to this invention. It will also be appreciated that from this disclosure functional benefits may be conferred on plants by means of introduction into plants and expression of these genes in such plants. Methods known in the art may be utilized for this purpose. Thus, for example, those skilled in the art will appreciate that the methods, for example, for achieving the expression of the RHD6 and RSL genes of this invention may be achieved according methods disclosed herein, and by methods, for example, disclosed in, but not limited to, EP0803572B1, which discloses the cloning and expression of the cpc gene, which, like the RHD6 and RSL genes of this invention, is also related to the control of root hair development in plants, albeit at a different stage of plant and root hair development.

In various aspects, the invention provides ROOT HAIR DEFECTIVE 6 (RHD6)-related polypeptides encoded by ROOT HAIR DEFECTIVE 6 (RHD6)-related genes and nucleic acid sequences described herein.

ROOT HAIR DEFECTIVE 6 (RHD6)-related polypeptides include both ROOT HAIR DEFECTIVE 6 (RHD6) polypeptides and ROOT HAIR DEFECTIVE 6-LIKE 1 (RSL1) polypeptides, and functional homologues thereof, as described herein. RHD6-related polypeptides include may be capable of complementing the rhd6 mutation upon expression in plants.

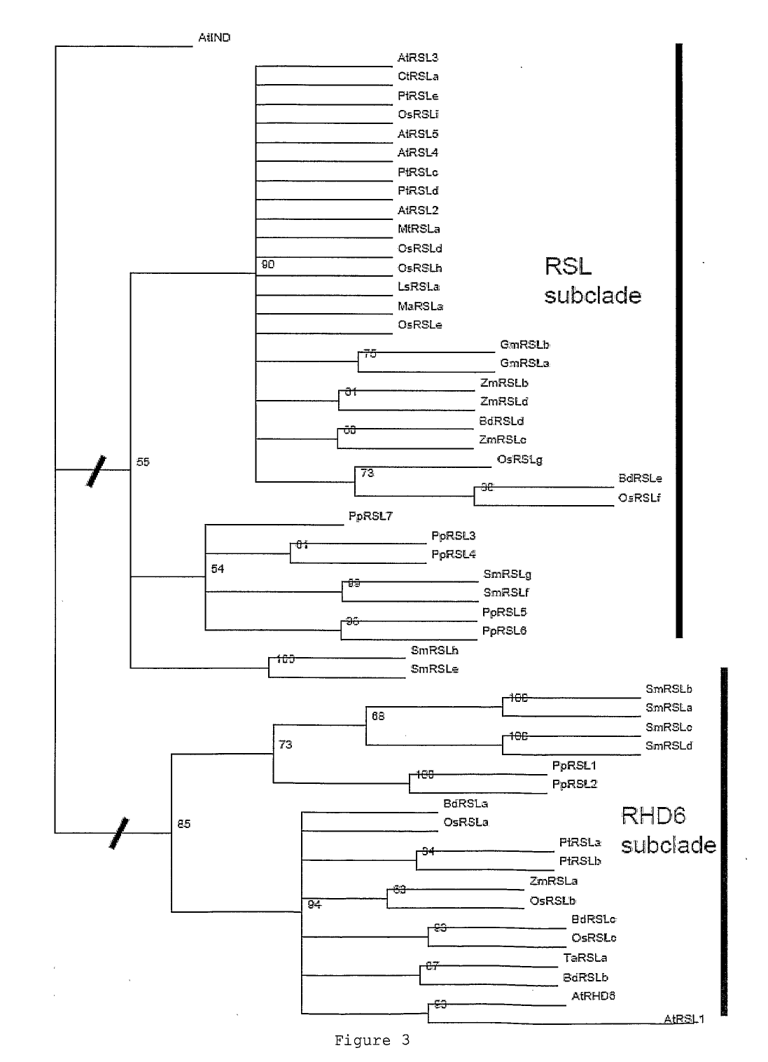

A ROOT HAIR DEFECTIVE 6 (RHD6)-related polypeptide may fall within the RHD6 clade comprising AtRHD6, AtRSL1, PpRSL1, PpRSL2, BdRSLb, TaRSLa, OsRSLc, BdRSLc, OsRSLb, ZmRSLa, PtRSLa, PrRSLb, OsRSLa, BdRSLa, SmRSLa, SmRSLb, SmRSLc and SmRSLd (the ROOT HAIR DEFECTIVE 6 (RHD6) clade) in a cladogram of protein sequences, for example using the sequences of AtIND and PpINDa as an outgroup (see FIG. 3).

Alternatively, ROOT HAIR DEFECTIVE 6 (RHD6)-related polypeptide may fall within the RSL clade comprising AtRSL3, CtRSLa, PtRSLe, OsRSLi, AtRSL5, AtRSL4, PtRSLc, PtRSLd, AtRSL2, MtRSLa, OsRSLd, OsRSLh, LsRSLa, MaRSLa, OsRSLe, GmRSLb, GmRSLa, ZmRSLb, ZmRSLd, BdRSLd, ZmRSLc, OsRSLg, BdRSLe, OsRSLf, PpRSL3, PpRSL4, PpRSL5, PpRSL6, PpRSL7, SmRSLg, SmRSLf, SmRSLh and SmRSLe (the ROOT HAIR DEFECTIVE SIX LIKE (RSL) clade) in a cladogram of protein sequences, for example using the sequences of AtIND and PpINDa as an outgroup (see FIG. 3).

A cladogram may be produced using conventional techniques. For example, a cladogram may be calculated using ClustalW to align the protein sequences, Phylip format for tree output, with 1000 bootstrap replicates and TreeViewX (version 0.5.0) for visualisation.

A suitable ROOT HAIR DEFECTIVE 6 (RHD6)-related polypeptide may comprise the amino acid sequence shown of SEQ ID NO: 1, 3, 5, 7, 9, 11, 13 to 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112 or 114 or may be a fragment or variant of one of these sequences which retains RHD6 activity.

In some preferred embodiments, the ROOT HAIR DEFECTIVE 6 (RHD6)-RELATED polypeptide may be a ROOT HAIR DEFECTIVE 6 (RHD6) polypeptide having the amino acid sequence of SEQ ID NO:1 (At1g66470; NP_{—}176820.1 GI: 15219658) or may be a fragment or variant of this sequence which retains RHD6 activity.

In other embodiments, the ROOT HAIR DEFECTIVE 6 (RHD6)-RELATED polypeptide may be a ROOT HAIR DEFECTIVE SIX LIKE (RSL) polypeptide having the amino acid sequence of any one of SEQ ID NOS: 5, 7, 9, and 11 or may be a fragment or variant of any of these sequences which retains RHD6 activity.

A ROOT HAIR DEFECTIVE 6 (RHD6)-RELATED polypeptide which is a variant a reference sequence set out herein, such as SEQ ID NO: 1, 3, 5, 7, 9, 11, 13 to 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112 or 114, may comprise an amino acid sequence which shares greater than 20% sequence identity with the reference amino acid sequence, preferably greater than 30%, greater than 40%, greater than 50%, greater than 60%, greater than 65%, greater than 70%, greater than 80%, greater than 90% or greater than 95%.

Particular amino acid sequence variants may differ from a RHD6-related polypeptide sequence as described herein by insertion, addition, substitution or deletion of 1 amino acid, 2, 3, 4, 5-10, 10-20 20-30, 30-50, or more than 50 amino acids.

Sequence identity is commonly defined with reference to the algorithm GAP (Wisconsin Package, Accelerys, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4.

Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm (Nucl. Acids Res. (1997) 25 3389-3402) may be used.

Sequence comparison may be made over the full-length of the relevant sequence described herein.

Certain domains of a RHD6-related polypeptide may show an increased level of identity with domains of a reference sequence, such as SEQ ID NO: 1, 3, 5, 7, 9, 11, 13 to 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112 or 114, relative to the RHD6-related polypeptide sequence as a whole. For example, a RHD6-related polypeptide may comprise one or more domains or motifs consisting of an amino acid sequence which has at least 70%, at least 75%, at least 80%, at least 90%, at least 95%, or at least 98% sequence identity or similarity, with an amino acid sequence selected from the group consisting of SEQ ID NOS: 13 to 25 or other RHD6-related polypeptide domain shown in tables 1 and 2.

In some preferred embodiments, a RHD6-related polypeptide may comprise one or more domains or motifs consisting of an amino acid sequence which is selected from the group consisting of SEQ ID NOS: 13 to 25 or other RHD6-related polypeptide domain shown in tables 1 and 2.

In various aspects, the invention provides ROOT HAIR DEFECTIVE 6 (RHD6)-related genes and nucleic acid sequences which encode ROOT HAIR DEFECTIVE 6 (RHD6)-related polypeptides, as described herein.

A nucleic acid encoding a RHD6-related polypeptide may comprise or consist of the nucleotide sequence of any one of SEQ ID NOS: 2, 4, 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115 or may be a variant or fragment of any one of these sequences which encodes a polypeptide which retains RHD6 activity.

In some preferred embodiments, a nucleic acid encoding a RHD6-related polypeptide may comprise or consist of the nucleotide sequence of SEQ ID NO: 2 or may be a variant or fragment of any one of these sequences which encodes a polypeptide which retains RHD6 activity.

A variant sequence may be a mutant, homologue, or allele of any one of SEQ ID NOS: 2, 4, 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115 and may differ from one of these sequences by one or more of addition, insertion, deletion or substitution of one or more nucleotides in the nucleic acid, leading to the addition, insertion, deletion or substitution of one or more amino acids in the encoded polypeptide. Of course, changes to the nucleic acid that make no difference to the encoded amino acid sequence are included. A nucleic acid encoding a RHD6-related polypeptide, which has a nucleotide sequence which is a variant of an RHD6-related nucleic acid sequence set out herein may comprise a sequence having at least 30% sequence identity with the nucleic acid sequence of any one of SEQ ID NOS: 2, 4, 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115, for example, preferably greater than 40%, greater than 50%, greater than 60%, greater than 65%, greater than 70%, greater than 80%, greater than 90% or greater than 95%. Sequence identity is described above.

A fragment or variant may comprise a sequence which encodes a functional RHD6-related polypeptide i.e. a polypeptide which retains one or more functional characteristics of the polypeptide encoded by the wild-type RHD6 gene, for example, the ability to stimulate or increase root hair number, growth or longevity in a plant or to complement the rhd6 mutation.

In other embodiments, a nucleic acid encoding a RHD6 polypeptide, which has a nucleotide sequence which is a variant of the sequence of any one of SEQ ID NOS: 2, 4, 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115 may selectively hybridise under stringent conditions with this nucleic acid sequence or the complement thereof.

Stringent conditions include, e.g. for hybridization of sequences that are about 80-90% identical, hybridization overnight at 42° C. in 0.25M Na₂HPO₄, pH 7.2, 6.5% SDS, 10% dextran sulfate and a final wash at 55° C. in 0.1×SSC, 0.1% SDS. For detection of sequences that are greater than about 90% identical, suitable conditions include hybridization overnight at 65° C. in 0.25M Na₂HPO₄, pH 7.2, 6.5% SDS, 10% dextran sulfate and a final wash at 60° C. in 0.1×SSC, 0.1% SDS.

An alternative, which may be particularly appropriate with plant nucleic acid preparations, is a solution of 5×SSPE (final 0.9 M NaCl, 0.05M sodium phosphate, 0.005M EDTA pH 7.7), 5× Denhardt's solution, 0.5% SDS, at 50° C. or 65° C. overnight. Washes may be performed in 0.2×SSC/0.1% SDS at 65° C. or at 50-60° C. in 1×SSC/0.1% SDS, as required.

Nucleic acids as described herein may be wholly or partially synthetic. In particular, they may be recombinant in that nucleic acid sequences which are not found together in nature (do not run contiguously) have been ligated or otherwise combined artificially. Alternatively, they may have been synthesised directly e.g. using an automated synthesiser.

The nucleic acid may of course be double- or single-stranded, cDNA or genomic DNA, or RNA. The nucleic acid may be wholly or partially synthetic, depending on design. Naturally, the skilled person will understand that where the nucleic acid includes RNA, reference to the sequence shown should be construed as reference to the RNA equivalent, with U substituted for T.

ROOT HAIR DEFECTIVE 6 (RHD6)-related polypeptides and nucleic acids may be readily identified by routine techniques of sequence analysis in a range of plants, including agricultural plants selected from the group consisting of Lithospermum erythrorhizon, Taxus spp, tobacco, cucurbits, carrot, vegetable brassica, melons, capsicums, grape vines, lettuce, strawberry, oilseed brassica, sugar beet, wheat, barley, maize, rice, soyabeans, peas, sorghum, sunflower, tomato, potato, pepper, chrysanthemum, carnation, linseed, hemp and rye.

A RHD6-related nucleic acid as described herein may be operably linked to a heterologous regulatory sequence, such as a promoter, for example a constitutive, inducible, root-specific or developmental specific promoter.

“Heterologous” indicates that the gene/sequence of nucleotides in question or a sequence regulating the gene/sequence in question, has been linked to the RHD6 related nucleic acid using genetic engineering or recombinant means, i.e. by human intervention. Regulatory sequences which are heterologous to an RHD6 related nucleic acid may be regulatory sequences which do not regulate the RHD6 related nucleic acid in nature or are not naturally associated with the RHD6 related nucleic acid. “Isolated” indicate that the isolated molecule (e.g. polypeptide or nucleic acid) exists in an environment which is distinct from the environment in which it occurs in nature. For example, an isolated nucleic acid may be substantially isolated with respect to the genomic environment in which it naturally occurs.

Many suitable regulatory sequences are known in the art and may be used in accordance with the invention. Examples of suitable regulatory sequences may be derived from a plant virus, for example the Cauliflower Mosaic Virus 35S (CaMV 35S) gene promoter that is expressed at a high level in virtually all plant tissues (Benfey et al, (1990) EMBO J 9: 1677-1684). Other suitable constitutive regulatory elements include the cauliflower mosaic virus 19S promoter; the Figwort mosaic virus promoter; and the nopaline synthase (nos) gene promoter (Singer et al., Plant Mol. Biol. 14:433 (1990); An, Plant Physiol. 81:86 (1986)). For example, RHD6-related genes such as AtRHD6, AtRSL1 and AtRSL4 may be expressed using constitutive promoters.

Constructs for expression of RHD6 and RSL genes under the control of a strong constitutive promoter (the 35S promoter) are exemplified below. Expression of AtRHD6, AtRSL1 and AtRSL4 from the 35S promoter is shown to modulate root hair development in plants without causing additional phenotypic changes.

However, those skilled in the art will appreciate that a wide variety of other promoters may be employed to advantage in particular contexts. Thus, for example, one might select an epidermal or root-specific promoter to ensure expression of these constructs only in roots. Suitable root-specific promoters are described for example in Qi et al PNAS (2006) 103(49) 18848-18853. For example, RHD6-related genes such as AtRSL2 and ATRSL3, may be expressed using root-specific promoters.

Alternatively, or in addition, one might select an inducible promoter. In this way, for example, in a cell culture setting, production of a particular gene product of interest may be enhanced or suppressed by induction of the promoter driving expression of the genes described herein. Inducible promoters include the alcohol inducible alc gene-expression system (Roslan et al., Plant Journal; 2001 October; 28(2):225-35) may be employed.

RHD6-related nucleic acid may be contained on a nucleic acid construct or vector. The construct or vector is preferably suitable for transformation into and/or expression within a plant cell.

A vector is, inter alia, any plasmid, cosmid, phage or Agrobacterium binary vector in double or single stranded linear or circular form, which may or may not be self transmissible or mobilizable, and which can transform prokaryotic or eukaryotic host, in particular a plant host, either by integration into the cellular genome or exist extrachromasomally (e.g. autonomous replicating plasmid with an origin of replication).

Specifically included are shuttle vectors by which is meant a DNA vehicle capable, naturally or by design, of replication in two different organisms, which may be selected from actinomyces and related species, bacteria and eukaryotic (e.g. higher plant, mammalia, yeast or fungal) cells.

A construct or vector comprising nucleic acid as described above need not include a promoter or other regulatory sequence, particularly if the vector is to be used to introduce the nucleic acid into cells for recombination into the genome.

Constructs and vectors may further comprise selectable genetic markers consisting of genes that confer selectable phenotypes such as resistance to antibiotics such as kanamycin, hygromycin, phosphinotricin, chlorsulfuron, methotrexate, gentamycin, spectinomycin, imidazolinones, glyphosate and d-amino acids.

Those skilled in the art are well able to construct vectors and design protocols for recombinant gene expression, in particular in a plant cell. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 3rd edition, Sambrook & Russell, 2001, Cold Spring Harbor Laboratory Press.

Those skilled in the art can construct vectors and design protocols for recombinant gene expression, for example in a microbial or plant cell. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 3rd edition, Sambrook et al, 2001, Cold Spring Harbor Laboratory Press and Protocols in Molecular Biology, Second Edition, Ausubel et al. eds. John Wiley & Sons, 1992. Specific procedures and vectors previously used with wide success upon plants are described by Bevan, Nucl. Acids Res. (1984) 12, 8711-8721), and Guerineau and Mullineaux, (1993) Plant transformation and expression vectors. In: Plant Molecular Biology Labfax (Croy R R D ed) Oxford, BIOS Scientific Publishers, pp 121-148.

When introducing a chosen gene construct into a cell, certain considerations must be taken into account, well known to those skilled in the art. The nucleic acid to be inserted should be assembled within a construct that contains effective regulatory elements that will drive transcription. There must be available a method of transporting the construct into the cell. Once the construct is within the cell membrane, integration into the endogenous chromosomal material either will or will not occur. Finally, the target cell type is preferably such that cells can be regenerated into whole plants.

Those skilled in the art will also appreciate that in producing constructs for achieving expression of the genes according to this invention, it is desirable to use a construct and transformation method which enhances expression of the RHD6 gene, the RSL gene or a functional homolog thereof. Integration of a single copy of the gene into the genome of the plant cell may be beneficial to minimize gene silencing effects. Likewise, control of the complexity of integration may be beneficial in this regard. Of particular interest in this regard is transformation of plant cells utilizing a minimal gene expression construct according to, for example, EP Patent No. EP 1 407 000 B1, herein incorporated by reference for this purpose.

Techniques well known to those skilled in the art may be used to introduce nucleic acid constructs and vectors into plant cells to produce transgenic plants with the properties described herein.

Agrobacterium transformation is one method widely used by those skilled in the art to transform woody plant species, in particular hardwood species such as poplar. Production of stable, fertile transgenic plants is now routine in the art: (Toriyama, et al. (1988) Bio/Technology 6, 1072-1074; Zhang, et al. (1988) Plant Cell Rep. 7, 379-384; Zhang, et al. (1988) Theor Appl Genet 76, 835-840; Shimamoto, et al. (1989) Nature 338, 274-276; Datta, et al. (1990) Bio/Technology 8, 736-740; Christou, et al. (1991) Bio/Technology 9, 957-962; Peng, et al. (1991) International Rice Research Institute, Manila, Philippines 563-574; Cao, et al. (1992) Plant Cell Rep. 11, 585-591; Li, et al. (1993) Plant Cell Rep. 12, 250-255; Rathore, et al. (1993) Plant Molecular Biology 21, 871-884; Fromm, et al. (1990) Bio/Technology 8, 833-839; Gordon-Kamm, et al. (1990) Plant Cell 2, 603-618; D'Halluin, et al. (1992) Plant Cell 4, 1495-1505; Walters, et al. (1992) Plant Molecular Biology 18, 189-200; Koziel, et al. (1993) Biotechnology 11, 194-200; Vasil, I. K. (1994) Plant Molecular Biology 25, 925-937; Weeks, et al. (1993) Plant Physiology 102, 1077-1084; Somers, et al. (1992) Bio/Technology 10, 1589-1594; WO92/14828; Nilsson, O. et al (1992) Transgenic Research 1, 209-220).

Other methods, such as microprojectile or particle bombardment (U.S. Pat. No. 5,100,792, EP-A-444882, EP-A-434616), electroporation (EP 290395, WO 8706614), microinjection (WO 92/09696, WO 94/00583, EP 331083, EP 175966, Green et al. (1987) Plant Tissue and Cell Culture, Academic Press), direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d)) may be preferred where Agrobacterium transformation is inefficient or ineffective, for example in some gymnosperm species.

Physical methods for the transformation of plant cells are reviewed in Oard, 1991, Biotech. Adv. 9: 1-11.

Alternatively, a combination of different techniques may be employed to enhance the efficiency of the transformation process, e.g. bombardment with Agrobacterium coated microparticles (EP-A-486234) or microprojectile bombardment to induce wounding followed by co-cultivation with Agrobacterium (EP-A-486233).

Following transformation, a plant may be regenerated, e.g. from single cells, callus tissue or leaf discs, as is standard in the art. Almost any plant can be entirely regenerated from cells, tissues and organs of the plant. Available techniques are reviewed in Vasil et al., Cell Culture and Somatic Cell Genetics of Plants, Vol I, II and III, Laboratory Procedures and Their Applications, Academic Press, 1984, and Weissbach and Weissbach, Methods for Plant Molecular Biology, Academic Press, 1989.

The particular choice of a transformation technology will be determined by its efficiency to transform certain plant species as well as the experience and preference of the person practising the invention with a particular methodology of choice. It will be apparent to the skilled person that the particular choice of a transformation system to introduce nucleic acid into plant cells is not essential to or a limitation of the invention, nor is the choice of technique for plant regeneration.

Other aspects of the invention relate to the modulation of plant root hair development using RHD6 related polypeptides and nucleic acids as described herein.

A method of modulating root hair development or altering the root hair phenotype in a plant may comprise;
increasing the expression of a RHD6-related polypeptide within cells of said plant relative to control plants.

Modulation of root hair development in a plant may include increasing one or more of: root-hair growth, number of root-hairs, length of root-hairs, rate of growth of root-hairs, and longevity of individual root-hairs on the plant.

RHD6-related polypeptides are described in more detail above.

Expression of an RHD6-related polypeptide may be increased by any suitable method. In some embodiments, the expression of a RHD6-related polypeptide may be increased by expressing a heterologous nucleic acid encoding the RHD6-related polypeptide within cells of said plant.

Suitable controls will be readily apparent to the skilled person and may include plants in which the expression of the RHD6-related polypeptide is not increased.

A method of producing a plant with altered root hair phenotype may comprise:
incorporating a heterologous nucleic acid which alters the expression of a RHD6-related polypeptide into a plant cell by means of transformation, and;
regenerating the plant from one or more transformed cells.

Suitable RHD6-related polypeptides are described in more detail above.

In some embodiments, the plant may be a plant whose roots are not naturally colonised by symbiotic fungi, such as Mycorrhizae. Plants whose roots are not naturally colonised by fungi include non-mycorrhizal plants such as Brassicas.

Plants for use in the methods described herein preferably lack mutations in RHD6-related genes. For example the plant may be a wild-type plant.

A plant with altered root hair phenotype produced as described above may show improved tolerance to nutrient-deficient growth conditions, increased production of phytochemicals and/or increased phytoremediation properties, such as absorption of heavy metals.

Nucleic acid encoding RHD6-related polypeptides and their expression in plants is described in more detail above.

In other embodiments, the expression of an RHD6-related polypeptide may be increased by increasing the expression of an endogenous nucleic acid encoding the RHD6-related polypeptide within cells of said plant.

The expression of an endogenous nucleic acid encoding the RHD6-related polypeptide within cells of said plant may be increased by recombinant means, such as the targeted insertion of regulatory factors,

The expression of an endogenous nucleic acid encoding the RHD6-related polypeptide within cells of said plant may be increased by non-recombinant means. For example, expression of the RHD6-related polypeptide may be increased in a plant by selective plant breeding methods which employ the RHD6-related amino acid or nucleic acid sequence as a molecular marker in order to produce a plant having an altered root hair phenotype, for example increased size, number or longevity of root hairs relative to controls.

A method of producing a plant having altered root hair phenotype may comprise:
providing a population of plants,
determining the amount of expression of a RHD6-related polypeptide as described herein in one or more plants in the population, and
identifying one or more plants in the population with increased expression of the RHD6-related polypeptide relative to other members of said population.

The identified plants may be further propagated or crossed, for example, with other plants having increased RHD6-related polypeptide expression or self-crossed to produce inbred lines. The expression of an RHD6-related polypeptide in populations of progeny plants may be determined and one or more progeny plants with reduced expression of the RHD6-related polypeptide identified.

The expression of an RHD6-related polypeptide in a plant may be determined by any convenient method. In some embodiments, the amount of expression of the RHD6-related polypeptide may be determined at the protein level. A method of producing a plant with altered root hair development may comprise:
providing a population of plants,
determining the amount of RHD6-related polypeptide in one or more plants of said population, and
identifying one or more plants in the population with increased amounts of an RHD6-related polypeptide relative to other members of said population.

The amount of RHD6-related polypeptide may be determined in one or more cells of the plant, preferably cells from a below-ground portion or tissue of the plant, such as the root.

The amount of RHD6-related polypeptide may be determined using any suitable technique. Conveniently, immunological techniques, such as Western blotting may be employed, using antibodies which bind, to the RHD6-related polypeptide and show little or no binding to other antigens in the plant. For example, the amount of an RHD6-related polypeptide in a plant cell may be determined by contacting a sample comprising the plant cell with an antibody or other specific binding member directed against the RHD6-related polypeptide, and determining binding of the RHD6-related polypeptide to the sample. The amount of binding of the specific binding member is indicative of the amount of RHD6-related polypeptide which is expressed in the cell.

In other embodiments, the expression of the RHD6-related polypeptide may be determined at the nucleic acid level. For example, the amount of nucleic acid encoding an RHD6-related polypeptide may be determined. A method of producing a plant having altered root hair development may comprise:
providing a population of plants,
determining the level or amount of nucleic acid, for example mRNA, encoding the RHD6-related polypeptide in a cell of one or more plants of said population, and,
identifying one or more plants in the population with increased amount of nucleic acid encoding an RHD6-related polypeptide relative to other members of said population.

The level or amount of encoding nucleic acid in a plant cell may be determined for example by detecting the amount of transcribed encoding nucleic acid in the cell. Numerous suitable methods for determining the amount of a nucleic acid encoding an RHD6-related polypeptide in a plant cell are available in the art, including, for example, Northern blotting or RT-PCR (see for example Molecular Cloning: a Laboratory Manual: 3rd edition, Sambrook & Russell (2001) Cold Spring Harbor Laboratory Press NY; Current Protocols in Molecular Biology, Ausubel et al. eds. John Wiley & Sons (1992); DNA Cloning, The Practical Approach Series (1995), series eds. D. Rickwood and B. D. Hames, IRL Press, Oxford, UK and PCR Protocols: A Guide to Methods and Applications (Innis, et al. 1990. Academic Press, San Diego, Calif.).

A suitable cell may be from a below-ground portion or tissue of the plant, such as the root.

A progeny plant identified as having increased RHD6-related polypeptide expression may be tested for altered root hair development relative to controls, for example increased growth, number or longevity of root hairs, or may be tested for other properties, such as increased resistance to nutrient deficient conditions, increased phytochemical production, increased phytoremediation properties or a constitutive low phosphate response.

A method of producing a plant having an altered root hair phenotype may comprise:
crossing a first and a second plant to produce a population of progeny plants;
determining the expression of a RHD6-related polypeptide in the progeny plants in the population, and
identifying a progeny plant in the population in which expression of the RHD6-related polypeptide is increased relative to controls.

A progeny plant having an altered root hair phenotype may show increased growth, number or longevity of root hairs relative to controls (e.g. other members of the population with a wild-type phenotype).

The identified progeny plant may be further propagated or crossed, for example with the first or second plant (i.e. backcrossing) or self-crossed to produce inbred lines.

The identified progeny plant may be tested for increased tolerance to nutrient-deficient conditions relative to controls.

Other aspects of the invention provide the use of an RHD6-related polypeptide or encoding nucleic acid as described herein as a marker for the selective breeding of a plant which has an altered root hair phenotype relative to control plants, and a method of selective breeding of a plant which has an altered root hair phenotype relative to control plants, which employs the RHD6-related amino acid or encoding nucleic acid sequence.

In some embodiments, plants having reduced expression of the RHD6-related polypeptide may be produced by random mutagenesis, followed by screening of mutants for reduced RHD6-related polypeptide expression. Suitable techniques are well known in the art and include Targeting Induced Local Lesions IN Genomes (TILLING). TILLING is a high-throughput screening technique that results in the systematic identification of non-GMO-derived mutations in specific target genes (Comai and Henikoff, The Plant Journal (2006) 45, 684-694 Till et al_BMC Plant Biol. 2007 Apr. 7, 19.

Those skilled in the art will also appreciate that, based on the genetic information disclosed herein, Targeted Induced Local Lesions IN Genomes (“TILLING”, e.g. utilizing PCR-based screening of plants generated through chemical mutagenesis (generally via ethyl methane sulfonate (EMS) treatment), often resulting in the isolation of missense and nonsense mutant alleles of the targeted gene(s); TILLING permits the high-throughput identification of mutations in target genes without production of genetically modified organisms and it can be an efficient way to identify mutants in a specific gene that might not confer a strong phenotype by itself), may be carried out to produce plants and offspring thereof with a change in the RHD6 or RSL gene, thereby permitting identification of plants with specific phenotypes relevant to plant root hair production.

A method of producing a plant having an altered root hair phenotype may comprise:
exposing a population of plants to a mutagen,
determining the expression of a RHD6-related polypeptide or nucleic acid in one or more plants in said population, and
identifying a plant with increased expression of the RHD6-related polypeptide relative to other members of said population.

Suitable mutagens include ethane methyl sulfonate (EMS).

Methods for determining the expression of RHD6-related polypeptide or nucleic acid in plants is described in more detail above.

The identified plant may be further tested for increased tolerance or resistance to low-nutrient conditions relative to controls, increased production of phytochemicals or increased phytoremediation.

A plant identified as having increased expression of the RHD6-related polypeptide relative to controls (e.g. other members of the population) may display increased growth, number or longevity of root hairs relative to the controls.

A plant produced or identified as described above may be sexually or asexually propagated or grown to produce off-spring or descendants. Off-spring or descendants of the plant regenerated from the one or more cells may be sexually or asexually propagated or grown. The plant or its off-spring or descendants may be crossed with other plants or with itself.

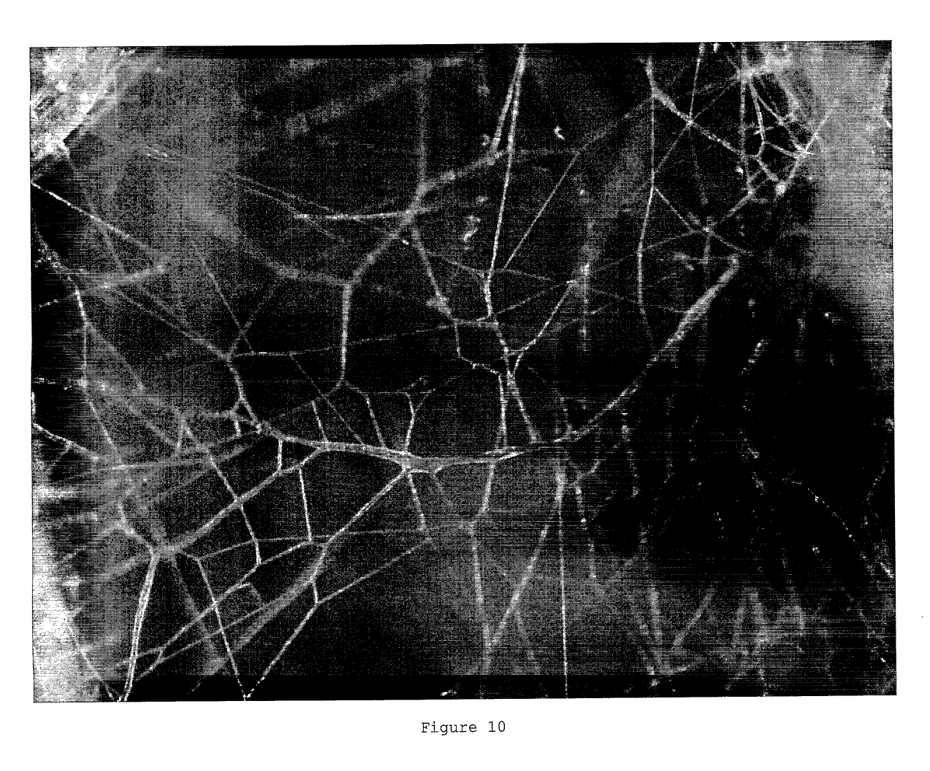

Expression of RHD6-related genes such as RSL4 is shown herein to produce a phenotype in which the root hairs display a fungus-like morphology. This morphology is characterised by extensive indeterminate masses of growing cells which resemble fungal like colonies (FIG. 10) and results in a greatly increased surface area of root hairs. This phenotype may confer significantly enhanced root uptake of phosphate and iron, which is largely limited by the length and surface area of the root hair.

By means of expression of the genes described herein in a plant, a plant may be made to exhibit enhanced absorption of otherwise less efficiently absorbed nutrients. Thus, for example, it is known in the art that phosphate and iron absorption from the soil is achieved primarily by plant root hairs. By enhancing the number, length, time of production or duration of survival of plant root hairs, by expression, overexpression, or targeted misexpression (expression in cells that otherwise may not produce root hairs) of RHD6-related genes, absorption of iron or phosphate or both, as well as other nutrients, may be enhanced. In particular, absorption may be increased by the fungus-like root hair morphology produced by overexpression of RHD6-related genes such as AtRSL4 (see FIG. 10). Thus, it has been shown in the literature that rhd6 mutants are compromised in their ability to absorb phosphate, see Plant Growth and Phosphorus Accumulation of Wild Type and Two Root Hair Mutants of Arabidopsis thaliana (Brassicaceae)“, Terence R. Bates and Jonathan P. Lynch, American Journal of Botany 87(7): 958-963. 2000. This effect may be reversed by supplementation with the functional gene described herein. Aspects of the invention would be of particular benefit, for example, in low-iron or low-phosphate containing soils, such as those found in China, sub-Saharan Africa and Australia

A method of improving the tolerance or resistance of a plant to nutrient deficient conditions may comprise;
increasing the expression of an RHD6-related polypeptide within cells of said plant relative to control plants.

Nutrient deficient conditions include conditions which contain levels of one or more nutrients such as nitrate, phosphate and/or iron, which are insufficient to fulfill the nutritional requirements of the wild-type plant. A wild-type plant subjected to nutrient deficient conditions may adopt a nutrient deficient phenotype, such as reduced growth resulting in greatly reduced yield and crop quality.

For example, the plant may show improved growth in soil which contains low levels of one or more nutrients such as nitrate, phosphate and/or iron, relative to control plants (i.e. plants in which RHD6-related polypeptide expression is unaltered).

Furthermore, phosphate deficiency increases the expression of RHD6-related polypeptides such as AtRHD6 and AtRSL1 in the root epidermis of a plant. Expression of a RHD6-related polypeptide in the root epidermis may therefore be useful in producing a constitutive “low phosphate” response in a plant.

The genes described herein may be utilized to achieve enhanced production of compounds of interest, including medicinally relevant compounds. Thus, for example, it is known that plant root hairs are responsible for production of antibiotic compounds. In nature, these compounds are secreted by plant roots and especially plant root hairs, to thereby modify or otherwise control the microflora and microfauna surrounding the plant roots. Production of these phytochemicals is enhanced in plants in which the number, length, duration of production, time of production and other characteristics of root hair development and growth may be modified at will according to the methods of this invention.

A method of increasing the production or secretion of a root-secreted phytochemical in a plant may comprise;
increasing the expression of a RHD6-related polypeptide within cells of a plant which secretes the phytochemical through its roots.

Root-secreted phytochemicals include shikonin (Brigham L A, et al Plant Physiol. 1999 February; 119(2):417-28) which may be produced by Lithospermum erythrorhizon, and paclitaxel, which may be produced by Taxus spp.

Heavy metals are an important environmental pollutant and may be removed by growing plants on contaminated soils. In phytoremediation or phytoextraction, plants absorb contaminating substances such as heavy metals from the soil and the plants are harvested at maturity, thereby removing these contaminants from the area. The long root hair phenotypes conferred by increased expression of RHD6-related polypeptides may enhance the phytoremediation properties of plant species.

A method of reducing the amount of a contaminating substance in soil comprising;
increasing the expression of a RHD6-related polypeptide within cells of a plant which absorbs the contaminating substance through its roots,
growing the plant or a descendent thereof in soil which comprises the contaminating substance such that the plant or descendent absorbs the contaminating substance from the soil, and
harvesting said plant or descendent thereof.

Contaminating substances include uranium, polychlorinated biphenyls, salt, arsenic and heavy metals such as cadmium, zinc and lead.

A plant suitable for use in the present methods is preferably a higher plant, for example an agricultural plant selected from the group consisting of Lithospermum erythrorhizon, Taxus spp, tobacco, cucurbits, carrot, vegetable brassica, melons, capsicums, grape vines, lettuce, strawberry, oilseed brassica, sugar beet, wheat, barley, maize, rice, soyabeans, peas, sorghum, sunflower, tomato, potato, pepper, chrysanthemum, carnation, linseed, hemp and rye.

In embodiments relating to phytochemical production, Lithospermum erythrorhizon and Taxus spp may be preferred.

In embodiments relating to phytoremediation, sunflower (Helianthus annuus), Chinese Brake fern, alpine pennycress (Thlaspi caerulescens), Indian mustard (Brassica juncea), Ragweed (Ambrosia artemisiifolia) Hemp Dogbane (Apocymun cannabinum) and Poplar may be preferred.

Another aspect of the invention provides a plant which is produced by a method described herein, wherein said plant shows altered root hair phenotype relative to controls.

For example, a plant may display increased growth, number or longevity of root hairs relative to controls (e.g. other members of the population with a wild-type phenotype).

A plant may display increased tolerance to nutrient deficient conditions and/or increased production of root-secreted phytochemicals.

Also provided is any part or propagule of such a plant, for example seeds, selfed or hybrid progeny and descendants.

A plant according to the present invention may be one which does not breed true in one or more properties. Plant varieties may be excluded, particularly registrable plant varieties according to Plant Breeders Rights.

In addition to a plant produced by a method described herein, the invention encompasses any clone of such a plant, seed, selfed or hybrid progeny and descendants, and any part or propagule of any of these, such as cuttings and seed, which may be used in reproduction or propagation, sexual or asexual. Also encompassed by the invention is a plant which is a sexually or asexually propagated off-spring, clone or descendant of such a plant, or any part or propagule of said plant, off-spring, clone or descendant.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof.

However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

All documents mentioned in this specification are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures described above and table described below.

Tables 1 and 2 show a sequence alignment of bHLH amino acid sequences (Heim et al. Mol. Biol. Evol. 2003) generated by ClustalW (http://www.ebi.ac.uk).

Table 3 shows % identities of RHD6-related proteins as determined by DNA Strider (Christain Mark, Center. d'Etudes de Saclay).

Table 4 shows relative identities of the bHLH domains of RHD6-related proteins to RHD6.

Table 5 shoes the correspondence between the names on the tree of FIG. 3 and the alignments of tables 1 and 2 with respective species and locus or GI accession number.

### EXAMPLES

Root hairs are highly polarised cells that increase the surface area of the plant that is in contact with the soil. They play important roles in nutrient acquisition and anchorage in those land plants that have roots^{1, 2}. Other tip growing cells such as rhizoids and caulonemal cells have a similar function in more basal groups of land plants that lack roots^{3, 4}. Here we identify and characterise two basic helix loop helix transcription factors that control the development of root hair cells in Arabidopsis sporophyte and show that their closest homologs in Physcomitrella patens are required for the development of both rhizoids and caulonemal cells in the gametophyte of this moss. This indicates that an ancient mechanism controls the development of functionally and morphologically similar but non-homologous cell types in these divergent groups of land plants. This suggests that the evolution of the land plant body over the past 475 million years^{5, 6} has resulted at least in part from the independent recruitment of genes from the gametophyte to the sporophyte.

Unless, stated otherwise, standard techniques were as follows:

RT-PCR

Total RNA (5 μg for A. thaliana and 1 μg for P. patens) was reverse transcribed with the Superscript First Strand synthesis system (Invitrogen, Carlsbad, USA) in a 20 μl reaction containing oligo d (T )12-18 primer. One μl of this product was used for PCR in 20 μl reactions containing primers described herein.

Pollen Growth Experiments

In vivo and in vitro pollen tube growth experiments were done as described previously (44, 45).

Southern Analysis of Inserts

Southern blots were performed with the DIG System for PCR labelling of DNA probes (Roche Diagnostics, Penzberg, Germany) according to manufacturer protocol Hybridization was done at 42° C. in DIG Easy Hyb hybridization buffer.

Arabidopsis Growth Conditions.

Arabidopsis thaliana (L.) Heyn. lines were grown vertically for 4 days on MS medium+2% sucrose solidified with 0.5% Phytagel at 24° C. under continuous illumination. For the cellophane disc experiment, the agar was overlaid with a cellophane disk (AA packaging, Preston, UK) before application of the seeds.

Enhancer Trapping and Cloning of the AtRHD6 Gene

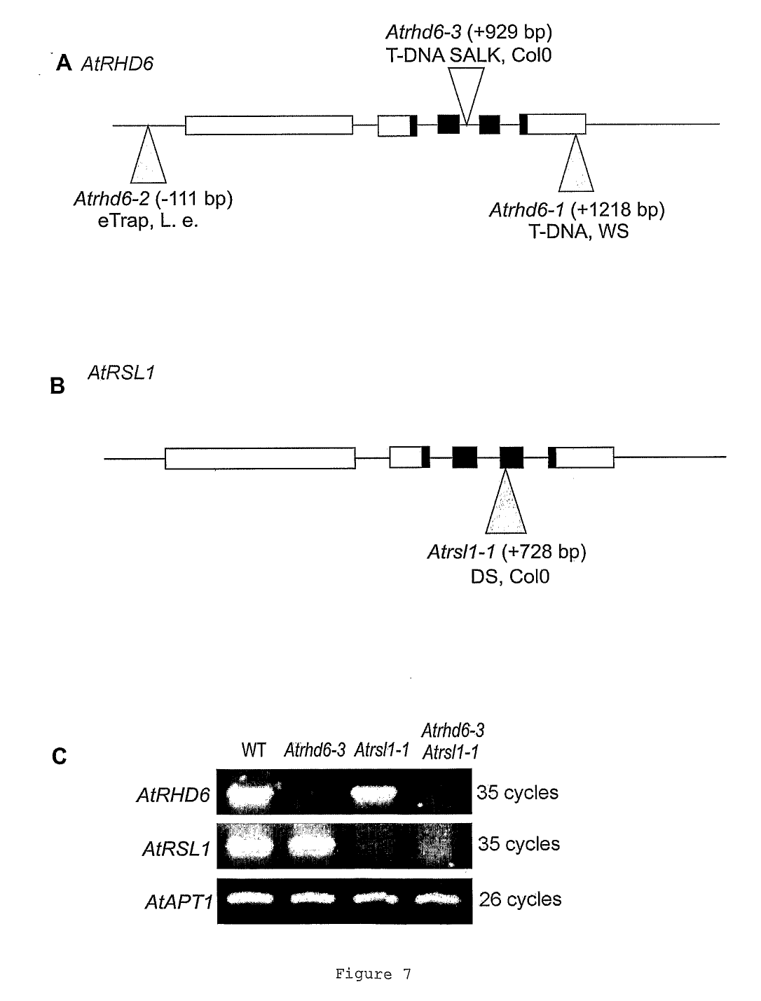

Atrhd6-2 is an enhancer trap line (1261) of Arabidopsis (ecotype Lansberg erecta) generated with the DsE element¹⁸ that was screened for root hairless phenotype and reporter gene expression in hair cells. Failure to complement Atrhd6-1⁷ indicated that line 1261 carries a mutation that is allelic to Atrhd6-1. The DNA sequence flanking the DsE element insertion was identified by inverse-PCR¹⁹. Genomic DNA of the 1261 line was digested by Sau3A I and subsequently ligated using T4 DNA ligase. The ligated DNA was used for PCR with Ds element-specific primers. This showed that the DsE is inserted 111 bp upstream the ATG site of At1g66470 gene (FIG. 7).

GUS Staining of Arabidopsis thaliana Roots and Embedding

Four-days-old seedlings were stained for 12 hr at 37° C. in 1 mM 5-bromo-4-chloro-3-indolyl-glucuronide, 0.5 mM potassium ferricyanide, 0.5 mM potassium ferrocyanide, and 10 mM sodium phosphate buffer (pH 7). Seedlings were embedding in Technovit 7100® resin (Kulzer GmbH, Germany) according to the manufacturer instructions and 10 μm transverse sections were taken from roots.

Identification of A. thaliana Mutants and Generation of Transgenic Plants

Verification of the T-DNA insertion sites in mutants used in this work (FIG. 7) was carried out by sequencing PCR fragments amplified with primers described herein. Atrhd6-3 (ecotype Columbia 0) correspond to the GABI-Kat line 475E09 (10). Atrsl1-1 (ecotype Columbia 0) corresponding to line WiscDsLox356A02 comes from the Biotechnology centre of the University of Wisconsin. cpc, wer, ttg1 and gl2 mutants have been described previously (32-35).

The genomic constructs AtRHD6p::GFP:AtRHD6 and AtRSL1p::GFP:AtRSL1 contain the promoter and 5′UTR of AtRHD6 or AtRSL1 upstream of the GFP coding sequence fused in N-terminal to the AtRHD6 or AtRSL1 coding region including introns and the AtRHD6 or AtRSL1 3′UTR with terminator. These constructs were generated using the Gateway system (Invitrogen, Carlsbad, USA). The AtRHD6 or AtRSL1 promoter+5′UTR and the AtRHD6 or AtRSLl coding region+3′UTR+terminator were amplified with PCR primers containing recombination sequences and cloned into pDONR P4-P1R and pDONR P2R-P3. A GATEWAY multisite reaction was then performed with the two resulting pDONR plasmids, the plasmid p207-GFP2.5 and the binary vector pGWBmultisite (destination vector). The binary vector pGWBmultisite was generated by replacing the R1-CmR-ccdB-R2 cassette of pGWB1 into R4-CmR-ccdB-R3 (pGWB1 is from Tsuyoshi Nakagawa, Shimane University, Japan). AtRHD6p::GFP:AtRHD6 and AtRSL1p::GFP:AtRSL1 were transformed respectively in Atrhd6-3 and in Atrhd6-3 Atrsl1-1 double mutant by floral dip (36) and transformants were selected on kanamycin (50 μg/ml) and hygromycin (50 μl/ml). Nine independent transgenic lines containing the AtRHD6p::GFP:AtRHD6 construct in the Atrhd6-3 background were obtained. They show different levels of complementation of the AtRhd6-hairless phenotype but all lines express the GFP in hair cells before the emergence of root hair. Five independent lines having the same GFP expression pattern and restore the Atrhd6-3 phenotype were obtained for AtRSL1p::GFP:AtRSL1 transformation in Atrhd6 Atrsl1.

For the p35S::PpRSL1 construct, the PpRSL1 coding sequence was amplified from protonema cDNA. This fragment was cloned between the BamHI and SalI sites of a modified pCAMBIA1300 plasmid containing the CaMV 35S promoter and the terminator of pea Rubisco small subunit E9 from 35S-pCAMBIA1301 cloned into its EcoRI and PstI sites (37). The p35S::PpRSL1 construct was transformed by floral dip in Atrhd6-3 and transformants were selected on hygromycin (50 μg/ml). Ten independent transgenic lines that complement the Atrhd6-3 hairless phenotype were obtained.

Physcomitrella Genes Isolation and Phylogenetic Analyses

Physcomitrella RSLs and the PpIND1 genome sequences where obtained from BLAST of the available genome sequence assembled into contigs (http://moss.nibb.ac.jp/). The splice sites were predicted with NetPlantGene 20 and the bHLH coding sequences were confirmed by RTPCR and sequencing. The full length coding sequence of PpRSL1 was obtained by sequencing EST clone pdp31414, provided by the RIKEN BioResource Center²¹ and the full length coding sequence of PpRSL2 was obtained by RT-PCR. Sequences have been deposited to GenBank as follows: PpRSL1 (EF156393), PpRSL2 (EF156394), PpRSL3 (EF156395), PpRSL4 (EF156396), PpRSL5 (EF156397) PpRSL6 (EF156398), PpRSL7 (EF156399) and PpIND1 (EF156400). The phylogenetic analysis was performed with PAUP* software as described previously²².

Physcomitrella Growth Conditions

The Gransden wild type strain of Physcomitrella patens (Hedw.) Bruch and Schimp 23 was used in this study. Cultures were grown at 25° C. and illuminated with a light regime of 16 h light/8 h darkness and a quantum irradiance of 40 μE m-2s-1. For the analysis of protonema phenotype, spores kept at 4° C. for at least 1 month were germinated in a 5 ml top agar (0.8%) plated on 9 cm Petri dish containing 25 ml of 0.8% agar overlaid with a cellophane disk (AA packaging, Preston, UK). Leafy gametophores were grown on 100 times diluted minimal media²⁴ supplemented with 5 mg/L NH4 tartrate and 50 mg/L Glucose.

Constructing Mutants in Physcomitrella Genes

The constructs for Physcomitrella transformation were made in plasmids pBNRF and pBHSNR. pBNRF carries a NptII gene driven by a 35S promoter cloned in the EcoRI site of pBilox, a derivative of pMCS5 (MoBiTec, Goettingen Germany) carrying two direct repeats of the loxP sites cloned in the XhoI-KpnI and BglII-SpeI sites. pBHSNR contains a AphIV gene driven by a 35S promoter clone between the 2 loxP sites of pBilox using SacI and NotI. pPpRSL1-KO was made by cloning PpRSL1 genomic fragment 1 in pBNRF digested with XbaI and XhoI and then cloning PpRSL1 genomic fragment 2 in the resulting plasmid digested with HpaI and AscI. pPpRSL2-KO was made by cloning PpRSL2 genomic fragment 1 in pBHSNR digested with MluI and SpeI and then cloning PpRSL2 genomic fragment 2 in the resulting plasmid digested with BamHI and HindIII.

PEG Transformation of Protoplasts

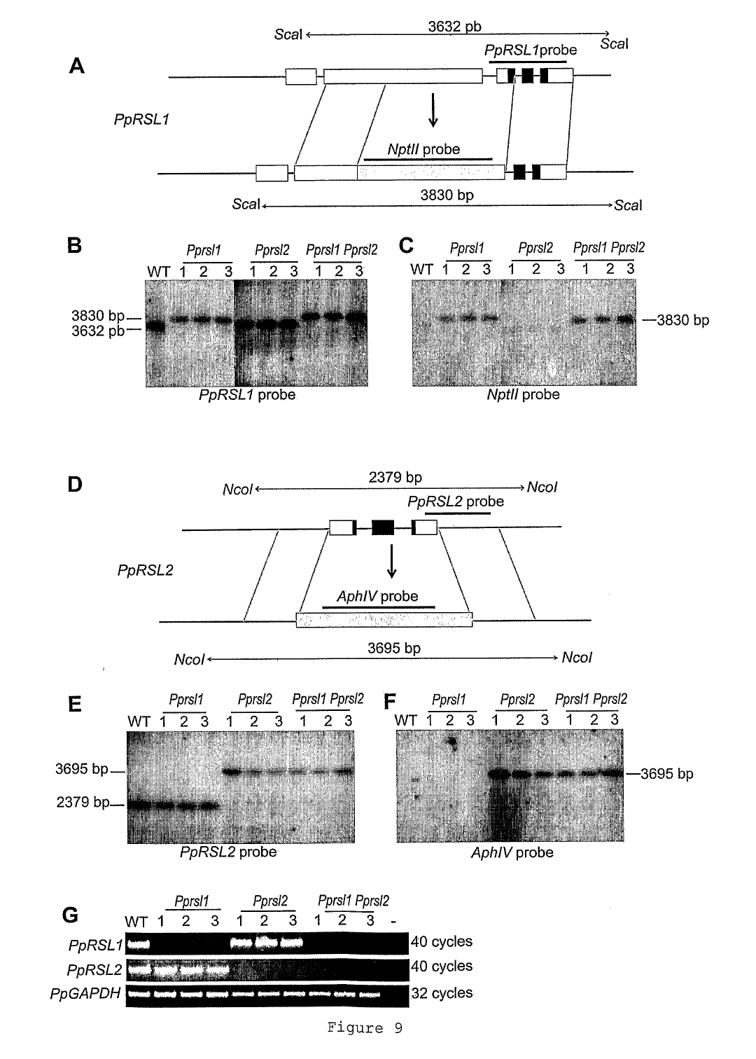

PEG transformation of protoplasts was done as described previously²⁵. pPpRSL1-KO was linearised with ScaI and SspI before protoplast transformation and transformants were selected on G418 (50 μl/ml). pPpRSL2-KO was linearised with BclI and SspI before protoplast transformation and transformants were selected on Hygromycin B (25 μl/ml). The Pprsl1 Pprsl2 double mutants were obtained by transformation of Pprsl1 line 1 with the pPpRSL2-KO construct. Stable transformants were first selected by PCR using primers flanking the recombination sites and then analysed by Southern blot and RT-PCR. For each transformation, three independent lines having the expected single insertion pattern and being RNA null mutants were selected (FIG. 9). In each case, the 3 transformants selected had the same phenotype.

Plants Overexpressing RHD6 or RSL Genes

For the 35S::RHD6 and RSL2, 3, and 4 constructs, the coding sequence of each gene was amplified from root cDNA with primers as listed below. This fragment was subcloned into a modified pCAMBIA1300 plasmid containing the CaMV 35S promoter and the terminator of pea Rubisco small subunit E9. All these overexpression constructs were transformed by floral dip in rhd6/rsl1 and transformants were selected on hygromycin (50 μl/ml)

35S::RHD6
CCAGGATCCATGGCACTCGTTAATGACCAT
CCAGTCGACTTAATTGGTGATCAGATTCGAA
35S::RSL2
CCAGGATCCATGGGAGAATGGAGCAACAA
CCAGTCGACTCATCTCGGTGAGCTGAGA
35S::RSL3
CGGGGTACC ATGGAAGCCATGGGAGAAT
CGCGGATCC TCATCTGGTCAGTGCATTGAG
35S::RSL4
CGGGGTACCATGGACGTTTTTGTTGATGGT
CGCGGATCCTCACATAAGCCGAGACAAAAG

Results

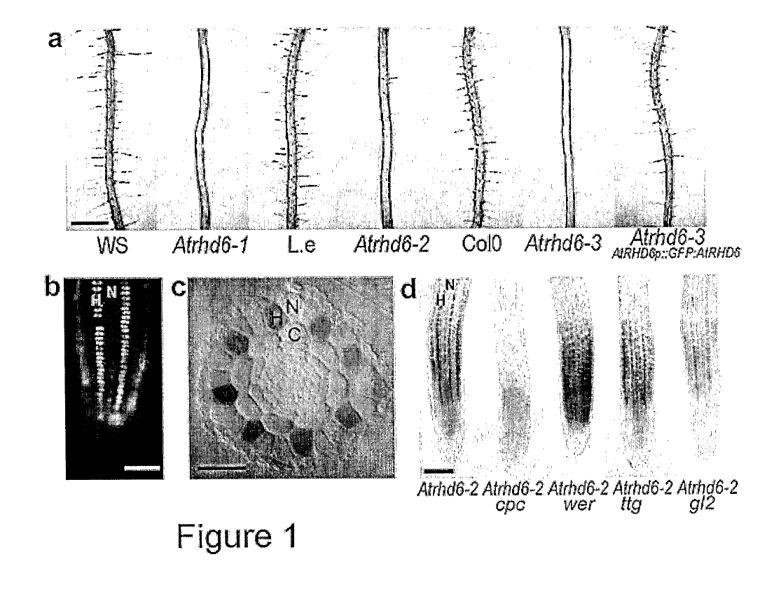

The Arabidopsis root epidermis is organised in alternate rows of hair forming cells (H cells) that produce a tip growing protuberance (root hairs) and rows of non-hair cells (N cells) that remain hairless. AtRHD6 (ROOT HAIR DEFECTIVE 6) positively regulates the development of H cells—Atrhd6 mutants develop few root hairs (FIG. 1a)⁷.

We cloned AtRHD6 using an enhancer trap line (Atrhd6-2) in which the GUS reporter gene is expressed in H cells but not in N cells (FIG. 1c, d, FIG. 7). AtRHD6 encodes the basic-Helix-loop-helix (bHLH) transcription factor At1g66470⁸. The identification of another independent allele (Atrhd6-3) with a similar phenotype and the complementation of the Atrhd6-3 mutation with a whole gene AtRHD6p::GFP:AtRHD6 fusion confirmed that the defect in root hair development observed in this mutant is due to mutation of At1g66470 (FIG. 1a). This complementing AtRHD6p::GFP:AtRHD6 fusion indicates that AtRHD6 protein accumulates in H cell nuclei in the meristem and elongation zones (FIG. 1b) but disappears before the emergence of the root hair. The spatial pattern of N cells and H cells in the Arabidopsis root epidermis is controlled by a transcriptional network including the positive regulator of H cell identity CPC and the negative regulators of H cell identity WER, TTG and GL2⁹.

To determine if AtRHD6 is regulated by these genes we analysed the promoter activity of the Atrhd6-2 enhancer trap in different mutant backgrounds. While the Atrhd6-2 enhancer trap expresses GUS in cells in the H position this expression spreads to the cells in the N position in the wer, ttg and gl2 mutant backgrounds indicating that WER, TTG and GL2 negatively, regulate transcription of AtRHD6 in the N position (FIG. 1d). No expression was observed in the cpc mutant indicating that CPC positively regulates AtRHD6 expression (FIG. 1d). Thus, AtRHD6 controls the development of root hair cells and acts downstream of the genes involved in epidermal pattern formation.

AtRHD6 is a member of sub family VIIIc of bHLH transcription factors that comprises five other members^{8, 10}. One of these genes, At5g37800, hereafter named RHD SIX-LIKE1 (AtRSL1), is very similar to AtRHD6 and these two genes may derive from a relatively recent duplication event⁸. This provides indication that AtRHD6 and AtRSL1 might have redundant functions. To determine if AtRSL1 is also required for toot hair development we identified a line (Atrsl1-1) carrying a complete loss of function mutation in the AtRSL1 gene and created the Atrhd6-3 Atrsl1-1 double mutant (FIG. 7).

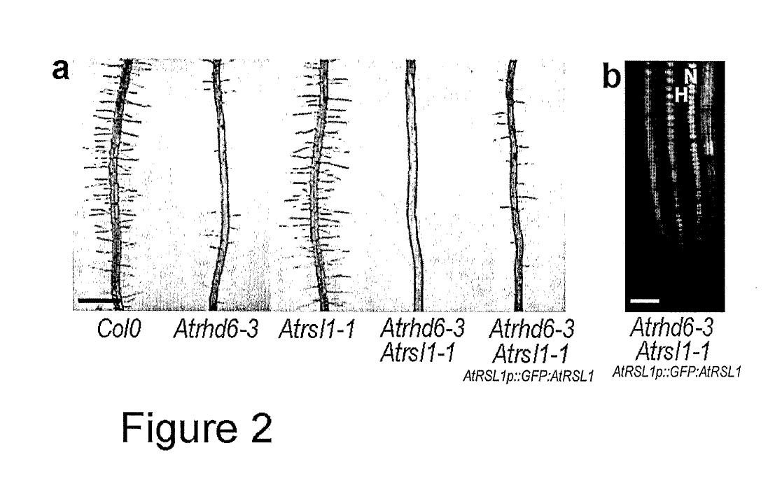

Because no new phenotypes were observed when these mutants were grown in our standard growth conditions, we grew them on the surface of cellophane discs, where small numbers of root hairs develop in the Atrhd6-3 single mutant (FIG. 2a). Plants homozygous for the Atrsl1-1 mutation had wild type root hair morphology when grown on cellophane discs (FIG. 2a). However, the Atrhd6-3 Atrsl1-1 double mutant did not develop root hairs, indicating that AtRHD6 and AtRSL1 have partially redundant functions in root hair development (FIG. 2a). Atrhd6-3 Atrsl1-1 double mutant plants carrying the genomic construct AtRSL1p::GFP:AtRSL1 displayed the AtRhd6-3-mutant phenotype, confirming that the extreme hairless phenotype of the Atrhd6-3 Atrsl1-1 double mutant is the result of a loss of function of both AtRHD6 and AtRSL1 genes (FIG. 2a). The complementing GFP:AtRSL1 fusion protein accumulates in hair cells nuclei in the meristem and elongation zones, indicating that AtRHD6 and AtRSL1 have similar expression patterns (FIG. 2b). These data indicate that AtRSL1 and AtRHD6 act together to positively regulate root hair development.

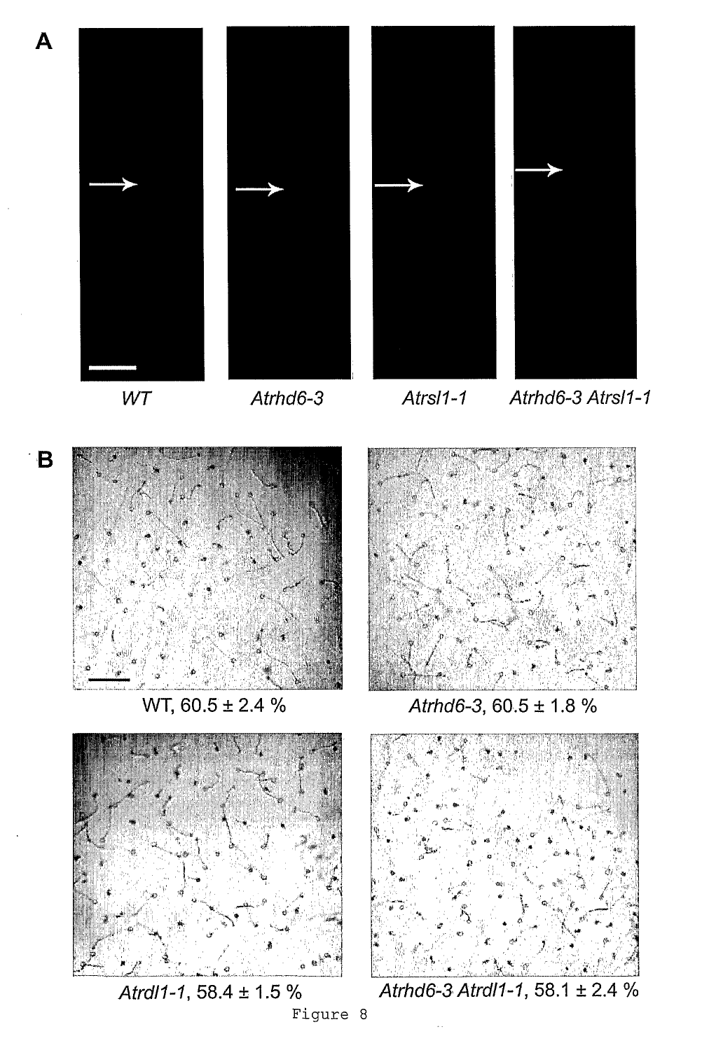

To determine if AtRHD6 and AtRSL1 are required for the development of the only other tip growing cell in flowering plants, the pollen tube, we characterised the phenotypes of pollen tubes in Atrhd6-3, Atrsl1-1 and Atrhd6-3 Atrsl1-1 mutants both in vitro and in vivo. We detected neither a defect in pollen tube growth nor in the segregation of mutant alleles in the F2 progeny of backcrosses to wild type (FIG. 8). No other defective phenotype was detected in any other part of Atrhd6-3, Atrsl1-1 or Atrhd6-3 Atrsl1-1 mutants.

Together these data indicate that AtRHD6 and AtRSL1 are bHLH transcription factors that are specifically required for the development of root hairs and act downstream of the genes that regulate epidermal pattern formation in the flowering plant Arabidopsis.

The most ancestral grade of land plants are the bryophytes—the earliest micro fossils of land plants from the middle Ordovician circa 475 Ma have bryophyte characteristics⁶. Bryophytes do not have roots but possess tip-growing cells that are morphologically similar to root hairs and fulfill rooting functions. In mosses, caulonemal cells increase the surface area of the filamentous protonema tissue in contact with the substrate and rhizoids anchor the leafy gametophore to their growth substrate^{3, 4} and both cell types are hypothesised to be involved in nutrient acquisition³. However, rhizoids and caulonema develop from the gametophyte of mosses whereas root hairs develop from the sporophyte of modern vascular plants. Thus, according to the current view that land plants evolved by the intercalation of a sporophytic generation from a haplontic algal ancestor followed by the progressive increase of size and complexity of the sporophyte in parallel to a reduction of the gametophyte^{11, 12}, neither rhizoids nor caulonema are homologous to root hairs.

To determine if the developmental mechanism that controls the development of root hairs in angiosperms also controls the development of non-homologous tip growing cells with a rooting function in bryophytes, we identified RHD6-LIKE genes from the moss Physcomitrella patens. We identified seven members of the AtRHD6 subfamily of bHLH genes from the publicly available Physcomitrella genomic sequence (http://moss.nibb.ac.jp/) providing indication that these genes have been conserved through the land plant evolution. These were designated Physcomitrella patens RHD6-LIKE 1 to 7 (PpRSL1 to PpRSL7). To analyse the relationship between Physcomitrella and Arabidopsis RSL genes we constructed phylogenetic trees by maximum parsimony. A strict consensus tree is presented in FIG. 3. This shows that AtRHD6, AtRSL1 and the two Physcomitrella PpRSL1 and PpRSL2 genes are closely related and together form a monophyletic clade (AtRHD6 clade) that is sister to the clade comprising all the other members of the subfamily (sister clade) (FIG. 3). This indicates that the AtRHD6 clade evolved before the separation of the bryophytes and the vascular plants from a common ancestor.

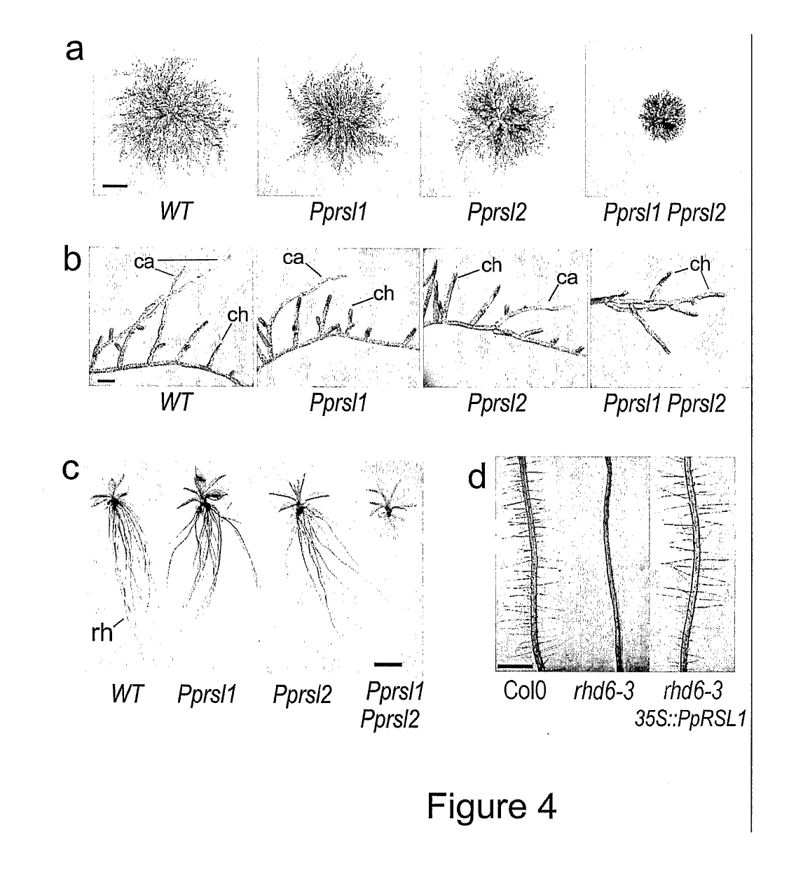

To characterize the function of the RHD6-LIKE genes in moss we constructed deletion mutants that lacked the function of PpRSL1 and PpRSL2 genes and determined if they develop morphological defects. Three independent RNA null mutants with single insertions in PpRSL1 and in PpRSL2 were made. Double mutants with single insertions into both genes were also generated (FIG. 9). The phenotypes of each of these mutants were then analysed. A haploid protonema develops upon germination of a wild type Physcomitrella spore 3. This filamentous tissue comprises two cell types, the chloronema and the caulonema (FIG. 4a, b). Chloronemal cells contain large chloroplasts and grow by a slow tip growth mechanism. Caulonemal cells are more elongated, contain few smaller chloroplasts, grow by rapid tip growth and are involved in the colonization of the substrate. Leafy gametophores usually develop from caulonema and are anchored to their substrate by tip growing multicellular rhizoids that are morphologically similar to caulonema (FIG. 4c). The Pprsl1 and Pprsl2 single mutants have slightly smaller and greener protonema cultures than WT and this phenotype is much stronger in the Pprsl1 Pprsl2 double mutant which produces small dark green protonema (FIG. 4a). Pprsl1 and Pprsl2 single mutants produce fewer caulonemal cells than the WT indicating that the greener protonema phenotype is the result of a defect in the development of caulonemal cells (FIG. 4b). No caulonemal cells develop in the Pprsl1 Pprsl2 double mutant and the protonema of this mutant comprises chloronemal cells only (FIG. 4b). In wild type plants gametophores develop from caulonema but in the Pprsl1 Pprsl2 double mutants the gametophores develop from chloronema, as previously observed in another caulonema defective mutant¹³. The gametophores of the Pprsl1 Pprsl2 double mutant develop few very short rhizoids (FIG. 4c). No other defective phenotypes were detected in the chloronema, in the leafy part of the gametophore or in the sporophyte in the single or double mutants. This indicates that PpRSL1 and PpRSL2 together regulate the development of caulonemal cells and rhizoids in the moss gametophyte. To determine if protein function is conserved across the land plants we performed a cross-species complementation experiment. Expression of PpRSL1 under the CaMV35S promoter in the Atrhd6-3 mutant resulted in the formation of wild type root hairs (FIG. 4d). Thus, the moss PpRSL1 gene can substitute for loss of AtRHD6 function in Arabidopsis. This indicates that the molecular function of PpRSL1 and AtRHD6 has been conserved since the divergence of seed plants and mosses from a common ancestor and suggests that the same molecular mechanism controls the development of Arabidopsis root hairs and Physcomitrella caulonema and rhizoids.

Plants were engineered to over-express RHD6 or RSL Genes using a constitutive promoter, as described above. The phenotype of these transformants is described below:

35S::RHD6

The deficient of root hair phenotype of rhd6/rsl1 can be rescued by the over-expression of RHD6. The transformants get longer root hair and higher percent of ectopic root hair (root hairs developed on the non hair cells) than col-0. A few root hairs can be observed on the hypocotyls.

Phosphate deficiency alters AtRHD6 and AtRSL1 gene expression. When grown in the presence of sufficient phosphate these genes are expressed in the meristem and elongation zone and transcription is down regulated in the regions where hairs form. When growing in conditions where phosphate is limiting, AtRHD6 and AtRSL1 are expressed in the root hair forming zone where they positively regulate the development of root hairs. This shows that phosphate deficiency promotes expression. Therefore, expressing high levels of AtRSL1 and AtRHD6 in the root epidermis results in a constitutive “low phosphate” response.

35S::RSL2 and 35S::RSL3

The rhd6/rsl1 plants harbouring 35S::RSL2 or 35S::RSL3 constructs also develop some root hair. The root hairs are longer than the col-0. Some transgenic lines showed swollen epidermal cells on the roots and hypocotyls. There are also some root hairs on the hypocotyls.

Over-expression of AtRSL2 and AtRSL3 using CaMV35S promoter in wild type plants results in the development of long root hairs. These hairs are not as long as those that form fungal like colonies upon over expression of AtRSL4. Plants over-expressing AtRSL2 and AtRSL3 develop stunted phenotypes. This may be due to expression in non-root hair cells.

35S::RSL4

The deficient of root hair phenotype of rhd6/rsl1 can also be partially complemented by introducing the over expression of RSL4. The transformants show longer root hair than col-0. A few root hairs were also detected on the hypocotyls, which is quite similar with that of RHD6 overexpression transformants.

Over-expression of AtRSL4 using CaMV35S promoter in wild type plants, causes the formation of long root hairs which can form extensive indeterminate growing masses of cells resembling fungal like colonies (FIG. 10). The root hair system of a plant overexpressing RSL4 is shown in FIG. 10. The root is surrounded by a mass of fungus-like cells, which resemble mycorrhizae, the nutrient scavanging fungi that form associations with roots. Furthermore, when the RSL4 is expressed by the 35S promoter, this phenotypic effect (long root hairs) was found to be restricted to the root hair cells. No defective phenotypes resulting from the RSL4 expression were observed elsewhere in the plant.

Plants overexpressing RHD6-related genes may therefore have increased nutrient uptake ability because of their increased surface area resulting from enhanced root hair growth. This effect may be marked in plants, such as Brassicas, which are devoid of mycorrhizae throughout their entire life cycle.

Here we show how closely related transcription factors control the development of tip growing cells that have a rooting function in the seed plant sporophyte and the bryophyte gametophyte. These data indicate that we have identified an ancient developmental mechanism that was present in the common ancestor of the mosses and vascular plants (tracheophytes). These genes will have been important for the invasion of land by plants when nutrient acquisition and anchorage to the solid substrate of the continental surface was necessary. The observation that rhizoids have been found on some of the oldest macro fossils of land plants is consistent with this view¹⁴⁻¹⁶.

Our results provide indication that RHD6-LIKE genes functioned in the haploid generation (gametophyte) of the early land plant life cycle which may have been bryophyte-like¹⁴, where they controlled the formation of cells with a rooting function. We propose that during the subsequent radiation of the land plants these genes were deployed in the development of the diploid generation (sporophyte) of vascular plants where they control the development of root hairs in angiosperms and we predict they control the development of root hairs and rhizoids in lycophytes (clubmosses and allies) and monilophytes (ferns and horse tails). It is likely that such independent recruitment of genes from haploid to diploid phases of the life cycle was in part responsible for the explosion in morphological diversity of the diploid stage of the life cycle (sporophyte) that occurred in the middle Palaeozoic when green plants colonised the continental surfaces of the planet¹⁷.

### REFERENCES

1. Carol, R. J. & Dolan, L. Philos. Trans. R. Soc. Lond. B Biol. Sci. 357, 815-21 (2002).

2. Gahoonia, T. S., Care, D. & Nielsen, N. E. Plant Soil 191, 181-188 (1997).

3. Duckett, J. G. et al. Protonemal morphogenesis. In Bryology for the twenty-first century (eds. Bates, J. W., Ashton, N. W. & Duckett, J. G.) 223-245 (British Bryological Society, 1998).

4. Sakakibara, K. et al. Development 130, 4835-4846 (2003).

5. Kenrick, P. & Crane, P. R. Nature 389, 33-39 (1997).

6. Wellman, C. H. et al Nature 425, 282-285 (2003).

7. Masucci, J. D. et al Plant Physiol. 106, 1335-1346 (1994).

8. Heim, M. A. et al. Mol. Biol. Evol. 20, 735-747 (2003).

9. Schiefelbein, J. Curr. Opin. Plant Biol. 6, 74-78 (2003).

10. Bailey, P. C. et al. Plant Cell 15, 2497-2501 (2003).

11. Blackwell, W. H. Bot. Rev. 69, 125-148 (2003).

12. Graham, L. E. et al Proc. Natl Acad. Sci. USA 97, 4535-4540 (2000).

13. Thelander, M. et al J. Exp. Bot. 56, 653-662 (2005).

14. Edwards, D. et al Nature 374, 635-636 (1995).

15. Kerp, H. et al New Data on Nothia aphylla Lyon 1964 ex El-Saadawy et Lacey 1979, a Poorly Known Plant from the Lower Devonian Rynie Chert. In Plants Invade the Land (eds. Gensel, P. & Edwards, D.) 52-82 (Columbia University Press, New York, 2001).

16. Kerp, H. et al T. Roy. Soc. Edin-Earth 94, 411-428 (2004).

17. Davis, P. & Kenrick, P. Fossil Plants (The Natural History. Museum, London, 2004).

18. Sundaresan, V. et al. Genes Dev. 9, 1797-1810 (1995).

19. Long, D. et al. Mol. Gen. Genet. 241, 627-636 (1993).

20. Hebsgaard, S. M. et al. Nucleic Acids Res. 24, 3439-3452 (1996).

21. Nishiyama, T. et al. PNAS. USA 100, 8007-8012 (2003).

22. Harrison, C. J. & Langdale, J. A. Plant J. 45, 561-572 (2006).

23. Ashton, N. W. & Cove, D. J. Mol. Gen. Genet. 154, 87-95 (1977).

24. Ashton, N. W. et al Planta 144, 427-435 (1979).

25. Schaefer, D. G. & Zryd, J. P. Plant J. 11, 1195-1206 (1997).

26. Liljegren, S. J. et al. Cell 116, 843-853 (2004).

27. Schiefelbein, J. W. et al The Plant Cell, 2, 235-243 (1990).

28. V. Sundaresan et al., Genes Dev. 9, 1797 (1995).

29. J. D. Masucci et al Plant Physiol. 106, 1335 (1994).

30. D. Long et al., Mol. & Gen. Genet. 241, 627 (1993).

31. M. G. Rosso et al., Plant Mol. Biol. 53, 247 (2003).

32. M. E. Galway et al., Dev. Biol. 166, 740 (1994).

33. M. M. Lee, J. Schiefelbein, Cell 99, 473 (1999).

34. J. D. Masucci, J. W. Schiefelbein, Plant Cell 8, 1505 (1996).

35. T. Wada et al Science 277, 1113 (1997).

36. S. J. Clough, A. F. Bent, Plant J. 16, 735 (1998).

37. K. K. Yi et al., Plant Physiol. 138, 2087 (2005).

38. S. M. Hebsgaard et al., Nucleic Acids Res. 24, (Sep. 1, 1996).

39. T. Nishiyama et al. PNAS USA 100, 8007 (2003).

40. C. J. Harrison, J. A. Langdale, Plant J. 45, 561 (2006).

41. N. W. Ashton, D. J. Cove, Mol. & Gen. Genet. 154, 87 (1977).

42. N. W. Ashton, N. H. Grimsley, D. J. Cove, Planta 144, 427 (1979).

43. D. G. Schaefer, J. P. Zryd, Plant J. 11, 1195 (1997).

44. L. M. Fan et al J. Exp. Bot. 52, 1603 (2001).

45. E. Ryan, et al New Phytol. 138, 49 (1998).

TABLE 1

| | | |
|---|---|---|
| AtRHD6 | TSPKDPQSLAAKNRRERISERLKILQELVPNGTKVDLVTMLEKAISYVKFLQVQVKVLATDEFWPAQ | 67 |
| AtRSL1 | TSPKDPQSLAAKNRRERISERLKVLQELVPNGTKVDLVTMLEKAIGYVKFLQVQVKVLAADEFWPAQ | 67 |
| PpRSL1 | GSANDPQSIAARVRRERISERLKVLQALIPNGDKVDMVTMLEKAISYVQCLEFQIKMLKNDSLWPKA | 67 |
| PpRSL2 | GSANDPQSIAARVRRERISERLKVLQALIPNGDKVDMVTMLEKAITYVQCLELQIKMLKNDSIWPKA | 67 |
| PpRSL5 | GSATDPQSVYARHRREKINERLKSLQNLVPNGAKVDIVTMLDEAIHYVKFLQNQVELLKSDELWIYA | 67 |
| PpRSL6 | GSATDPQSVYARHRREKINERLKNLQNLVPNGAKVDIVTMLDEAIHYVKFLQTQVELLKSDEFWMFA | 67 |
| PpRSL3 | GSATDPQSVYARHRREKINERLKTLQHLVPNGAKVDIVTMLDEAIHYVQFLQLQVTLLKSDEYWMYA | 67 |
| PpRSL4 | GSATDPQSVHARARREKIAERLRKLQHLIPNGGKVDIVTMLDEAVEYVQFLKRQVTLLKSDEYWMYA | 67 |
| PpRSL7 | GSATDPQSVYARHRREKINERLKTLQRLVPNGEQVDIVTMLEEAIHFVKFLEFQLELLRSDDRWMFA | 67 |
| At4g33880 | GAATDPQSLYARKRRERINERLRILQNLVPNGTKVDISTMLEEAVHYVKFLQLQIKLLSSDDLWMYA | 67 |
| At2g14760 | GAATDPQSLYARKRRERINERLRILQHLVPNGTKVDISTMLEEAVQYVKFLQLQIKLLSSDDLWMYA | 67 |
| At1g27740 | GTATDPQSLYARKRREKINERLKTLQNLVPNGTKVDISTMLEEAVHYVKFLQLQIKLLSSDDLWMYA | 67 |
| At5g43175 | GIASDPQSLYARKRRERINDRLKTLQSLVPNGTKVDISTMLEDAVHYVKFLQLQIKLLSSEDLWMYA | 67 |
| AtIND | RISDDPQTVVARRRRERISEKIRILKRIVPGGAKMDTASMLDEAIRYTKFLKRQVRILQPHSQIGAP | 67 |
| PpIND1 | RISKDPQSVAARHRRERISDRIRVLQRLVPGGTKMDTASMLDEAIHYVKFLKLQLQVCDTCNLVPVD | 67 |
| | • ***:: *: ***:* :::: *: ::*•* ::* :**:•*: :•: *: *: : • | |

TABLE 2

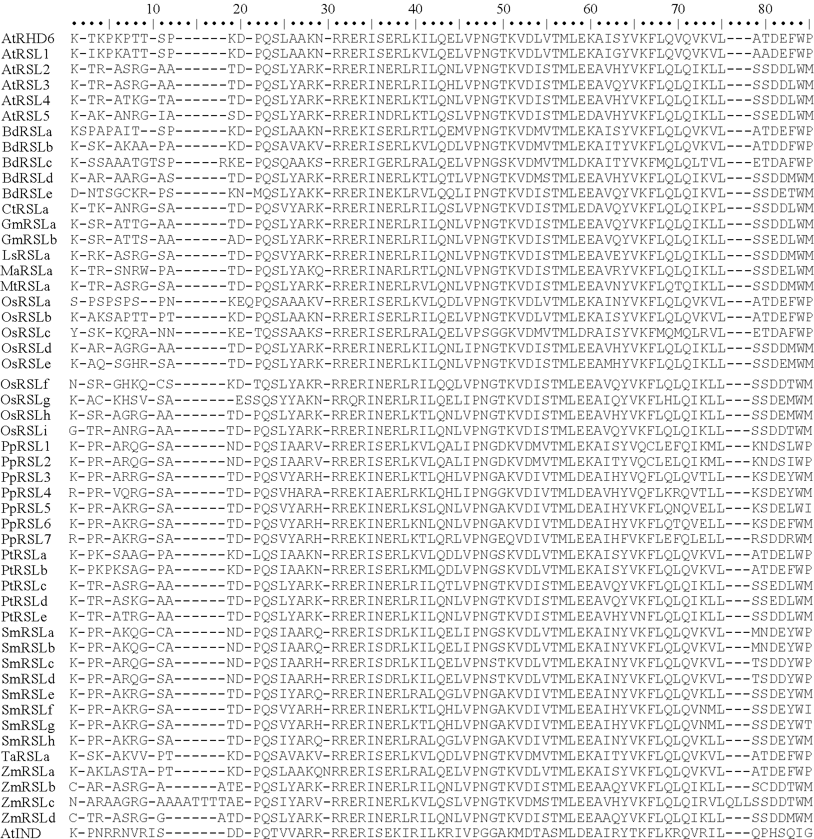

TABLE 3

| | | % Identity with RHD6 | |
|---|---|---|---|
| Gene | Database reference | over full length | over bHLH domain |
| RHD6 | At1g66470 | 100 | 100 |
| RSL1 | At5g37800 | 58.8 | 95.5 |
| RSL2 | At4g33880 | 19.1 | 75 |
| RSL3 | At2g14760 | 22.7 | 75 |
| RSL4 | At1g27740 | 23.2 | 73.1 |
| RSL5 | At5g43175 | 22.3 | 73.1 |

TABLE 4

| | Sequence Identity | | Sequence Identity |
|---|---|---|---|
| Protein | AtRHD6 | Protein | AtRHD6 |
| AtRHD6 | 1 | PpRSL1 | 0.625 |
| AtRSL1 | 0.93 | PpRSL2 | 0.611 |
| AtRSL2 | 0.611 | PpRSL3 | 0.583 |
| AtRSL3 | 0.611 | PpRSL4 | 0.513 |
| AtRSL4 | 0.597 | PpRSL5 | 0.597 |
| AtRSL5 | 0.583 | PpRSL6 | 0.611 |
| BdRSLa | 0.835 | PpRSL7 | 0.527 |
| BdRSLb | 0.763 | PtRSLa | 0.791 |
| BdRSLc | 0.675 | PtRSLb | 0.847 |
| BdRSLd | 0.611 | PtRSLc | 0.597 |
| BdRSLe | 0.569 | PtRSLd | 0.597 |
| CtRSLa | 0.625 | PtRSLe | 0.611 |
| GmRSLa | 0.611 | SmRSLa | 0.708 |
| GmRSLb | 0.597 | SmRSLb | 0.708 |
| LsRSLa | 0.611 | SmRSLc | 0.722 |
| MaRSLa | 0.611 | SmRSLd | 0.722 |
| MtRSLa | 0.611 | SmRSLe | 0.625 |
| OsRSLa | 0.78 | SmRSLf | 0.625 |
| OsRSLb | 0.902 | SmRSLg | 0.625 |
| OsRSLc | 0.652 | SmRSLh | 0.625 |
| OsRSLd | 0.597 | TaRSLa | 0.777 |
| OsRSLe | 0.638 | ZmRSLa | 0.835 |
| OsRSLf | 0.597 | ZmRSLb | 0.561 |
| OsRSLg | 0.602 | ZmRSLc | 0.493 |
| OsRSLh | 0.611 | ZmRSLd | 0.589 |
| OsRSLi | 0.597 | AtIND | 0.424 |

TABLE 5

| Name | Species | Locus name | GI accession |
|---|---|---|---|
| AtRHD6 | | AT1G66470 | |
| AtRSL1 | | AT5G37800 | |
| AtRSL2 | Arabidopsis thaliana | AT4G33880 | |
| AtRSL3 | | AT2G14760 | |
| AtRSL4 | | AT1G27740 | |
| AtRSL5 | | AT5G43175 | |
| BdRSLa | | | |
| BdRSLb | Brachypodium | N/A | |
| BdRSLc | distachyon | | |
| BdRSLd | | | |
| BdRSLe | | | |
| CtRSLa | Carthamus tinctorius | | 125399878 |
| GmRSLa | Glycine max | | 26056905 |
| GmRSLb | | | 15663066 |
| LsRSLa | Lactuca saligna | | 83790803 |
| MaRSLa | Musa acuminata | | 102139852 |
| MtRSLa | Medicago truncatula | | 92870204 |
| OsRSLa | | Os01g02110 | |
| OsRSLb | | Os02g48060 | |
| OsRSLc | | Os06g30090 | |
| OsRSLd | Oryza sativa ssp | Os03g10770 | |
| OsRSLe | japonica | Os03g42100 | |
| OsRSLi | | Os07g39940 | |
| OsRSLf | | Os11g41640 | |
| OsRSLg | | Os12g32400 | |
| OsRSLh | | Os12g39850 | |
| PpRSL1 | | | 140084326 |
| PpRSL2 | | | 140084333 |
| PpRSL3 | | | |
| PpRSL4 | Physcomitrella patens | | |
| PpRSL5 | | | |
| PpRSL6 | | | |
| PpRSL7 | | | |
| PtRSLa | | | |
| PtRSLb | | | |
| PtRSLc | Populus trichocarpa | | |
| PtRSLd | | | |
| PtRSLe | | N/A | |
| SmRSLa | | | |
| SmRSLh | | | |
| SmRSLb | | | |
| SmRSLc | Selaginella | | |
| SmRSLd | moelendorfii | | |
| SmRSLe | | | |
| SmRSLf | | | |
| SmRSLg | | | |
| TaRSLa | Triticum aestivum | | |
| ZmRSLa | Zea mays | AZM4_60871 | |
| ZmRSLb | | AZM4_70092 | |
| ZmRSLc | | AZM4_91750 | |
| ZmRSLd | | AZM4_86104 | |

Primers

A, Amplification and Sequencing of the Insertion Sites of A. thaliana Mutants

rhd6-1 insertion site
AtRHD6-A: 5′-GGATTGATTTAATTACCATATTTAT-3′
LB2: 5′-CAAGTATCAAACGATGTG-3′
rhd6-2 insertion site (inverse PCR)
DL3: 5′-CACCG GTACCGACCGTTACCGACCG-3′
Ds3I2: 5′-TACCGGTACCGAAAACGAACGGGA-3′
rhd6-3 insertion site
AtRHD6-B: 5′-GTTCCCAATGGCACCAAGGTACA-3′
GABI-LB: 5′-CCCATTTGGACGTGAATGTAGACAC-3′
rsl1-1 insertion site
AtRSL1-A: 5′-CGTGTGGACCGACGTCTGA
JL-202: 5′-CATTTTATAATAACGCTGCGGACATCTAC-3′

B, Construction of the pRHD6::GFP-RHD6::RHD6t Plasmid

Amplification of the AtRHD6 promoter+5′UTR fragment from BAC F28G11

RHD6prom-attB4F:
5′-GGGACAACTTTGTATAGAAAAGTTGTTCTCAAAGAGGGACAAGACCA
AAGCCCATGAC-3′
RHD6prom-attB1R:
5′-GGGGACTGCTTTTTTGTACAAACTTGCTAGACACTAATAAGTTTGAT
AAGTGATTTTTTGT-3′

Amplification of the AtRHD6 coding region+3′UTR+terminator fragment from BAC F28G11

RHD6term-attB2F:
5′-GGGGACAGCTTTCTTGTACAAAGTGGCCATGGCACTCGTTAATGACC
ATCCCAACGAGA-3′
RHD6term-attB3R:
5′-GGGGACAACTTTGTATAATAAAGTTGCTGATAAATCGAGATCTTAGG
TATGTCGTCC-3′

C, Construction of the pRSL1::GFP-RSL1::RSL1t Plasmid

Amplification of the AtRSL1 promoter+5′UTR fragment from Col0 genomic DNA

RSL1prom-attB4F:
5′-GGGGACAACTTTGTATAGAAAAGTTGTGATAATGGATTGGAGAAAAA
TTAAAG-3′
RSL1prom-attB1R:
5′-GGGGACTGCTTTTTTGTACAAACTTGTATTGCAATGTTCGTTAATGA
GTGAC-3′

Amplification of the AtRSL1 coding region+3′UTR+terminator fragment from Col0 genomic DNA

RSL1term-attB2F:
5′-GGGGACAGCTTTCTTGTACAAAGTGGGTAATTACATCTCAACCCCAA
ATTCTT-3′
RSL1term-attB3R:
5′-_ACAACTTTGTATAATAAAGTTGATGTATAATTTCCGAAGATGCTTA
AAA-3′

D, Construction of the p35S::PpRSL1 Plasmid

Amplification of RSL1 coding sequence from Col0 cDNA

35SPpRDL1 F: 5′-CCAGGATCCATGGCAGGTCCAGCAGGA-3′
35SPpRDL2 R: 5′-CCAGTCGACTTAGTCAGCAGAAGGCTGATT-3′

E, Construction of the pPpRSL1-KO Plasmid

Amplification of PpRSL1 fragment 1 from P. patens WT DNA

PpRSL1KO 1 F:
5′-CCTCTAGAAGTACTTGTGATCCACAGCCTA-3′
PpRSL1KO 1 R:
5′-GGCTCGAGCCGTACTGGGTGGTTTG-3′

Amplification of PpRSL1 fragment 2 from P. patens WT DNA

PpRSL1KO 2 F:
5′-CCGTTAACTTCTACATGTTGCGTTATTTATGGT-3′
PpRSL1KO 2 R:
5′-CCGGCGCGCCAATATTTATATAAATAAGCATAATACACTTCGA-3′

F, Construction of the pPpRSL2-KO Plasmid

Amplification of PpRSL2 fragment 1 from P. patens WT DNA

PpRSL2KO 1 F:
5′-GCAACGCGTGGGTTTGATCAAAGACGGAA-3′
PpRSL2KO 1 R:
5′-GCTACTAGTCGTCAACCTAACCCAAACAT-3′

Amplification of PpRSL2 fragment 2 from P. patens WT DNA

PpRSL2KO 2 F:
5′-GAGGGATCCGTGAGGTGAAAGCAGTGAAA-3′
PpRSL2KO 2 R:
5'-CCAAAGCTTAGGCCTGTGAACTCGGACA-3'

G, Amplification of Dig Labelled Probes for Southern Blots

Amplification of the PpRSL1 probe from P. patens WT DNA

| | |
|---|---|
| PpRSL1 probe F: | 5′-GCTGCTAGGGTAACATAAACATTCTT-3′ |
| PpRSL1 probe R: | 5′-CTGGACACTGGAATGAACCTA-3′ |

Amplification of NptII (NEOMYCIN PHOSPHOTRANSFERASE II) probe from pBNRF

| | |
|---|---|
| NptII probe F: | 5′-CCCATGGAGTCAAAGATTCA-3′ |
| NptII probe R: | 5′-CCGCGAATTCGAGCTCGGT-3′ |

Amplification of the PpRSL2 probe from P. patens WT DNA

| | |
|---|---|
| PpRSL2 probe F: | 5′-CCCAAATATGCATTTTTAATCTTT-3′ |
| PpRSL2 probe R: | 5′-GCGACAATCCAGCAGCCTCTAT-3′ |

Amplification of AphIV (AMINOGLYCOSIDE PHOSPHOTRANSFERASE IV) probe from pBHSNR

| | |
|---|---|
| AphIV probe F: | 5′-GTAGGAGGGCGTGGATATGT-3′ |
| AphIV probe R: | 5′-CGAGTGCTGGGGCGT-3′ |

H, Amplification of PpRSL2 Coding Sequence from Protonema cDNA

| | |
|---|---|
| PpRSL2 RT-PCR F: | 5′-GGGACCTCAAGGATGCAGCA-3′ |
| PpRSL2 RT-PCR R: | 5′-CGAACTCAATAACGTCAGGA-3′ |

I, RT-PCRs

| RT-PCR of AtAPT1 | |
|---|---|
| (ADENINE PHOSPHORIBOSYLTRANSFERASE 1) | |
| AtAPT1 RT-PCR F: | 5′-TCCCAGAATCGCTAAGATTGCC-3′ |
| AtAPT1 RT-PCR R: | 5′-CCTTTCCCTTAAGCTCTG-3′ |

RT-PCR of AtRHD6

| | |
|---|---|
| AtRHD6 RT-PCR F: | 5′-CTCACACGGGAGAGAGCA-3′ |
| AtRHD6 RT-PCR R: | 5′-CTTCGATTCTTGGCTGCTA-3′ |

RT-PCR of AtRSL1

| | |
|---|---|
| AtRSL1 RT-PCR F: | 5′-GCCTAGCAGCCAAGAACCGAA-3′ |
| AtRSL1 RT-PCR R: | 5′-CTCATCGGCTGCAAGTACCTTA-3′ |

RT-PCR of PpRSL1

| | |
|---|---|
| PpRSL1 RT-PCR F: | 5′-CTGGTTGGTTAGGAGATCTTGCAT-3′ |
| PpRSL1 RT-PCR R: | 5′-GTTGTAATTTGGTCCATTTCTGCT-3′ |

RT-PCR of PpRSL2

| | |
|---|---|
| PpRSL2 RT-PCR F: | 5′-GGGACCTCAAGGATGCAGCA-3′ |
| PpRSL2 RT-PCR R: | 5′-CGAACTCAATAACGTCAGGA-3′ |

RT-PCR of PpGAPDH
(GLYCERALDEHYDE 3-PHOSPHATE DEHYDROGENASE)

| | |
|---|---|
| PpGAPDH RT-PCR F: | 5′-GAGATAGGAGCATCTGTACCGCTTGT-3′ |
| PpGAPDH RT-PCR R: | 5′-CGCATGGTGGGATCGGCT-3′ |

SEQUENCES

RHD6 amino acid sequence (At1g66470; NP_176820.1 GI: 15219658 SEQ ID NO: 1)
MALVNDHPNETNYLSKQNSSSSEDLSSPGLDQPDAAYAGGGGGGGSASSSSTMNSDHQQH
QGFVFYPSGEDHHNSLMDFNGSSFLNFDHHESFPPPAISCGGSSGGGGFSFLEGNNMSYG
FTNWNHQHHMDIISPRSTETPQGQKDWLYSDSTVVTTGSRNESLSPKSAGNKRSHTGEST
QPSKKLSSGVTGKTKPKPTTSPKDPQSLAAKNRRERISERLKILQELVPNGTKVDLVTML
EKAISYVKFLQVQVKVLATDEFWPAQGGKAPDISQVKDAIDAILSSSQRDRNSNLITN
RHD6 nucleotide sequence (NM_105318.2 GI: 30697352 SEQ ID NO: 2)
atggcactcgttaatgaccatcccaacgagaccaattacttgtcaaaacaaaattcctcc
tcttccgaagatctctcctcgccgggactggatcagccagatgcagcttatgccggtgga
ggaggaggaggaggctcggcttcgagcagtagcacgatgaattcagatcatcaacaacat
caggggtttgtattttacccatccggtgaagatcatcacaactctttgatggatttcaac
ggatcatcatttcttaactttgatcatcacgagagctttcctcctccagccataagctgt
ggtggtagtagcggtgggggcggcttctccttcttggagggcaacaacatgagctacggc
ttcacaaactggaatcatcaacatcatatggatattattagccctagatccaccgaaact
ccccaaggccagaaagactggttatattctgattcaactgttgtaaccactggttctaga
aacgagtctctttcgcctaaatccgctggaaacaaacgttctcacacgggagagagcact
caaccgtcgaagaaactgagtagcggtgtgaccggaaagaccaagcctaagccaacaact
tcacctaaagatccacaaagcctagcagccaagaatcgaagagaaaggataagtgaacgt
ctcaagatattgcaagaacttgttcccaatggcaccaaggttgatttggtgacaatgctt
gaaaaggctattagttatgtcaagttccttcaagtacaagttaaggtattagcgaccgat
gagttttggccggctcaaggaggaaaagctcctgacatttctcaagttaaagacgccatt
gatgccattctctcctcatcacaacgagacaggaattcgaatctgatcaccaattaa
RSL1 amino acid sequence (At5g37800 SEQ ID NO: 3)
MSLINEHCNE RNYISTPNSS EDLSSPQNCG LDEGASASSS STINSDHQNN QGFVFYPSGE
TIEDHNSLMD FNASSFFTFD NHRSLISPVT NGGAFPVVDG NMSYSYDGWS HHQVDSISPR
VIKTPNSFET TSSFGLTSNS MSKPATNHGN GDWLYSGSTI VNIGSRHEST SPKLAGNKRP
FTGENTQLSK KPSSGTNGKI KPKATTSPKD PQSLAAKNRR ERISERLKVL QELVPNGTKV
DLVTMLEKAI GYVKFLQVQV KVLAADEFWP AQGGKAPDIS QVKEAIDAIL SSSQRDSNST
RETSIAE
RSL1 nucleotide sequence (SEQ ID NO: 4)
atgtcactcattaacgaacattgcaatgagcgtaattacatctcaaccccaaattcttca
gaagatctctcttcaccacagaattgcggattagacgaaggagcttcagcttcaagcagt
agcaccataaattctgatcatcaaaataatcaagggtttgtgttttacccttccggggaa
accattgaagatcataattctttgatggatttcaatgcttcatcattcttcacctttgat
aatcaccgaagccttatctctcccgtgaccaacggtggtgccttcccggtcgtggacggg
aacatgagttacagctatgatggctggagtcatcatcaagtggatagtattagccctaga
gtcatcaaaactccaaatagctttgaaacaacgagcagttttggattgacttcaaactcc
atgagtaaaccggccacaaaccatggaaatggagactggttatactctggttcaactatt
gtaaacatcggttcaaggcacgagtccacgtcccctaaactggctggcaataaacggcct
ttcacgggagagaacacacaactttcaaagaagccgagtagcggtacgaatggaaagatc
aagcctaaggcaacaacttcacctaaagatccacaaagcctagcagccaagaaccgaaga
gaaaggataagcgaacgcctcaaggtattgcaagaacttgtaccgaatggtaccaaggtg
gatttggtaactatgcttgagaaagcaattggctatgtaaagtttcttcaagtacaagtt
aaggtacttgcagccgatgagttttggccggcacaaggagggaaagctccggacatttct
caagttaaagaagctattgacgcaatcctctcatcatcacaacgagatagtaactcaact
agagaaacaagtatagcagaataa
RSL2 amino acid sequence (At4g33880; SEQ ID NO: 5)
MEAMGEWSNN LGGMYTYATE EADFMNQLLA SYDHPGTGSS SGAAASGDHQ GLYWNLGSHH
NHLSLVSEAG SFCFSQESSS YSAGNSGYYT VVPPTVEENQ NETMDFGMED VTINTNSYLV
GEETSECDVE KYSSGKTLMP LETVVENHDD EESLLQSEIS VTTTKSLTGS KKRSRATSTD
KNKRARVNKR AQKNVEMSGD NNEGEEEEGE TKLKKRKNGA MMSRQNSSTT FCTEEESNCA
DQDGGGEDSS SKEDDPSKAL NLNGKTRASR GAATDPQSLY ARKRRERINE RLRILQNLVP
NGTKVDISTM LEEAVHYVKF LQLQIKLLSS DDLWMYAPIA FNGMDIGLSS PR
RSL2 nucleotide sequence (SEQ ID NO: 6)
atggaagccatgggagaatggagcaacaacctcggaggaatgtacacttatgcaaccgag
gaagccgatttcatgaaccagcttctcgcctcttatgatcatcctggcaccggctcatcc
tccggcgcagcagccagtggtgaccaccaaggcttgtattggaaccttggttctcatcac
aaccaccttagcctcgtgtctgaagccggtagcttctgtttctctcaagagagcagcagc
tacagcgctgggaacagcggatattacaccgttgttccacccacggttgaagagaaccaa
aatgagacaatggactttgggatggaagatgtgaccatcaatacaaactcataccttgtt
ggtgaggagacaagtgagtgtgacgttgagaaatactcttctggaaagactcttatgcct
ttggaaaccgtagtggagaaccacgatgacgaggaaagcttgttgcaatctgagatctct
gtgactactacaaaatctctcaccggctccaaaaagagatcccgtgccacatctactgat
aaaaacaagagagcaagagtgaataagagggcccagaagaacgtagagatgagtggggat
aacaatgaaggagaagaggaagaaggagagacgaagttgaagaaaagaaagaatggggca
atgatgagtagacagaactcaagcaccactttctgtacggaggaagaatcaaactgcgct
gatcaagacggtggaggagaagactcatcctctaaggaagatgatccctcaaaggccctc
aacctcaatggtaaaacaagagccagtcgtggtgcagccaccgatcctcaaagcctctat
gcaaggaaaagaagagaaaggattaacgagagactaaggattttacaaaatctcgtcccc
aatggaacaaaggtcgatattagtacaatgcttgaggaagcagttcattacgtcaaattt
ttgcagctccaaattaagttattgagctctgatgatctatggatgtatgcgccgattgct
ttcaatgggatggacattggtctcagctcaccgagatga
RSL3 amino acid sequence (At2g14760; SEQ ID NO: 7)
MEAMGEWSTG LGGIYTEEAD FMNQLLASYE QPCGGSSSET TATLTAYHHQ GSQWNGGFCF
SQESSSYSGY CAAMPRQEED NNGMEDATIN TNLYLVGEET SECDATEYSG KSLLPLETVA
ENHDHSMLQP ENSLTTTTDE KMFNQCESSK KRTRATTTDK NKRANKARRS QKCVEMSGEN
ENSGEEEYTE KAAGKRKTKP LKPQKTCCSD DESNGGDTFL SKEDGEDSKA LNLNGKTRAS
RGAATDPQSL YARVDISTML EEAVQYVKFL QLQIKRLLAI GTNHRNRSIP LWTARNRQIS
KAHSRKRLRL RAVAKIIWSD EMTRFLLELI TLEKQAGNYR GKSLIEKGKE NVLVKFKKRF
PITLNWNKVK NRLDTLKKQY EIYPAKLRSH PLRFIPLLDV VFRDETVVVE ESWQPRRGVH
RRAPVLDLSD SECPNNNGDE REDLMQNRER DHMRPPTPDW MSQTPMENSP TSANSDPPFA
SQERSSTHTQ VKNVSRNRKR KQNPADSTLD RIAATMKKI
RSL3 nucleotide sequence (SEQ ID NO: 8)
atggaagccatgggagaatggagcaccggcctaggcggaatatatacagaggaagctgac
tttatgaatcagctccttgcctcctatgagcaaccttgtggcggttcatcttcagagaca
accgccacactcacggcctaccaccaccagggttctcaatggaatggtggcttttgcttc
tctcaggagagcagtagttatagtggttactgcgcggcgatgccacggcaagaagaagat
aacaatgggatggaggacgcgacaatcaacacgaacttgtaccttgttggtgaagagaca
agtgaatgtgatgcgacggaatactccggtaaaagcctcttgcctttggagactgtcgca
gaaaaccacgaccatagtatgctacagcctgagaactccttgaccacgaccactgatgag
aaaatgttcaaccaatgtgagagttcaaagaagaggacgcgtgccacaacaactgataag
aacaagagagccaacaaggcacgaaggagccagaaatgcgtagagatgagtggcgaaaat
gaaaatagcggcgaagaagaatatacggagaaggctgcggggaagagaaagaccaaacca
cttaagccgcaaaagacttgttgttcggatgacgaatcaaacggtggagacactttcttg
tccaaagaagatggcgaggactctaaggctctcaacctcaacggcaagactagggccagc
cgcggcgcggccacagatcctcaaagcctttacgcaaggaaaagaagagagaggataaac
gagaggctaaggattttgcaacatctcgtccctaatggaacaaaggttgatattagcacg
atgttggaagaagcagtacaatacgtcaaatttctacagctccaaattaagttattgagc
tctgatgatctatggatgtatgcgcctattgcttacaacggaatggacattggccttgac
ctaaaactcaatgcactgaccagatga
RSL4 amino acid sequence (At1g27740; SEQ ID NO: 9)
MDVFVDGELE SLLGMFNFDQ CSSSKEERPR DELLGLSSLY NGHLHQHQHH NNVLSSDHHA
FLLPDMFPFG AMPGGNLPAM LDSWDQSHHL QETSSLKRKL LDVENLCKTN SNCDVTRQEL
AKSKKKQRVS SESNTVDESN TNWVDGQSLS NSSDDEKASV TSVKGKTRAT KGTATDPQSL
YARKRREKIN ERLKTLQNLV PNGTKVDIST MLEEAVHYVK FLQLQIKLLS SDDLWMYAPL
AYNGLDMGFH HNLLSRLM
RSL4 nucleotide sequence (SEQ ID NO: 10)
atggacgtttttgttgatggtgaattggagtctctcttggggatgttcaactttgatcaa
tgttcatcatctaaagaggagagaccgcgagacgagttgcttggcctctctagcctttac
aatggtcatcttcatcaacatcaacaccataacaatgtcttatcttctgatcatcatgct
ttcttgctccctgatatgttcccatttggtgcaatgccgggaggaaatcttccggccatg
cttgattcttgggatcaaagtcatcacctccaagaaacgtcttctcttaagaggaaacta
cttgacgtggagaatctatgcaaaactaactctaactgtgacgtcacaagacaagagctt
gcgaaatccaagaaaaaacagagggtaagctcggaaagcaatacagttgacgagagcaac
actaattgggtagatggtcagagtttaagcaacagttcagatgatgagaaagcttcggtc
acaagtgttaaaggcaaaactagagccaccaaagggacagccactgatcctcaaagcctt
tatgctcggaaacgaagagagaagattaacgaaaggctcaagacactacaaaaccttgtg
ccaaacgggacaaaagtcgatataagcacgatgcttgaagaagcggtccattacgtgaag
ttcttgcagcttcagattaagttgttgagctcggatgatctatggatgtacgcaccattg
gcttacaacggcctggacatggggttccatcacaaccttttgtctcggcttatgtga
RSL5 amino acid sequence (At5g43175; SEQ ID NO: 11)
MENEAFVDGELESLLGMFNFDQCSSNESSFCNAPNETDVFSSDDFFPFGTILQSNYAAVL
DGSNHQTNRNVDSRQDLLKPRKKQKLSSESNLVTEPKTAWRDGQSLSSYNSSDDEKALGL
VSNTSKSLKRKAKANRGIASDPQSLYARKRRERINDRLKTLQSLVPNGTKVDISTMLEDA
VHYVKFLQLQIKLLSSEDLWMYAPLAHNGLNMGLHHNLLSRLI
RSL5 nucleotide sequence (SEQ ID NO: 12)
atggagaatgaagcttttgtagatggtgaattggagtctcttttggggatgttcaacttt
gatcaatgttcatctaacgaatcgagcttttgcaatgctccaaatgagactgatgttttc
tcttctgatgatttcttcccatttggtacaattctgcaaagtaactatgcggccgttctt
gatggttccaaccaccaaacgaaccgaaatgtcgactcaagacaagatctgttgaaacca
aggaagaagcaaaagttaagctcggaaagcaatttggttaccgagcctaagactgcttgg
agagatggtcaaagcctaagcagttataatagttcagatgatgaaaaggctttaggttta
gtgtctaatacatcaaaaagcctaaaacgcaaagcgaaagccaacagagggatagcttcc
gatcctcagagcctatacgctaggaaacgaagagaaaggataaacgataggctaaagaca
ttgcagagcctagttcctaatgggacaaaggtcgatataagcacaatgctggaagatgct
gtccattacgtgaagttcctgcagcttcaaatcaagctcttgagttcagaagatctatgg
atgtatgcacctcttgctcacaatggtctgaatatgggactacatcacaatcttttgtct
cggcttatttaa
AtRHD6 bHLH amino acid sequence (SEQ ID NO: 13)
TSPKDPQSLAAKNRRERISERLKILQELVPNGTKVDLVTMLEKAISYVKFLQVQVKVLATDEFWPAQ
AtRLD1 bHLH amino acid sequence (SEQ ID NO: 14)
TSPKDPQSLAAKNRRERISERLKVLQELVPNGTKVDLVTMLEKAIGYVKFLQVQVKVLAADEFWPAQ
PpRSL1 bHLH amino acid sequence (SEQ ID NO: 15)
GSANDPQSIAARVRRERISERLKVLQALIPNGDKVDMVTMLEKAISYVQCLEFQIKMLKNDSLWPKA
PpRSL2 bHLH amino acid sequence (SEQ ID NO: 16)
GSANDPQSIAARVRRERISERLKVLQALIPNGDKVDMVTMLEKAITYVQCLELQIKMLKNDSIWPKA
PpRSL5 bHLH amino acid sequence (SEQ ID NO: 17)
GSATDPQSVYARHRREKINERLKSLQNLVPNGAKVDIVTMLDEAIHYVKFLQNQVELLKSDELWIYA
PpRSL6 bHLH amino acid sequence (SEQ ID NO: 18)
GSATDPQSVYARHRREKINERLKNLQNLVPNGAKVDIVTMLDEAIHYVKFLQTQVELLKSDEFWMFA
PpRSL3 bHLH amino acid sequence (SEQ ID NO: 19)
GSATDPQSVYARHRREKINERLKTLQHLVPNGAKVDIVTMLDEAIHYVQFLQLQVTLLKSDEYWMYA
PpRSL4 bHLH amino acid sequence (SEQ ID NO: 20)
GSATDPQSVHARARREKIAERLRKLQHLIPNGGKVDIVTMLDEAVHYVQFLKRQVTLLKSDEYWMYA
PpRSL7 bHLH amino acid sequence (SEQ ID NO: 21)
GSATDPQSVYARHRREKINERLKTLQRLVPNGEQVDIVTMLEEAIHFVKFLEFQLELLRSDDRWMFA
At4g33880 bHLH amino acid sequence (SEQ ID NO: 22)
GAATDPQSLYARKRRERINERLRILQNLVPNGTKVDISTMLEEAVHYVKFLQLQIKLLSSDDLWMYA
At2g14760 bHLH amino acid sequence (SEQ ID NO: 23)
GAATDPQSLYARKRRERINERLRILQHLVPNGTKVDISTMLEEAVQYVKFLQLQIKLLSSDDLWMYA
At1g27740 bHLH amino acid sequence (SEQ ID NO: 24)
GTATDPQSLYARKRREKINERLKTLQNLVPNGTKVDISTMLEEAVHYVKFLQLQIKLLSSDDLWMYA
At5g43175 bHLH amino acid sequence (SEQ ID NO: 25)
GIASDPQSLYARKRRERINDRLKTLQSLVPNGTKVDISTMLEDAVHYVKFLQLQIKLLSSEDLWMYA
Physcomitrella RHD SIX LIKE 1 (PpRSL1) amino acid sequence (SEQ ID NO: 26;
ABO84930.1 GI: 140084327)
MAGPAGALWSTCDPQPIQQAEIFSGPDNQAGLMSFHVDTPFHWGSEPWALHSRSDDIALMSPSLVHDISPYDSVL
HLSGVSGDVQDLVCGNPKFRQSGQWGQSEFSYSVQDNMQDLLTNQFIPYNTSSLGLNHLSPNFTDLDCAPVYNDT
KAFGTVTHNRAVPSTNTQSAQHGSSSMVSSNRPITSTASPTTQYGGPRTPSQTTQYGGSSMVTNSMEMFASAAPQ
GIMTTSGLSGGCNSDLMHLPKRQHAHSLPPTTGRDLTASEVVSGNSISNISGVGSFNSSQKSSASVMMSPLAASS
HMHKAAAVSEELKMASFNPGPFVPTQKKQQHEQQDTMTSNRIWADKNNLGKISSSPIPIMGFEQSQQQSMSNSSP
VTSLGFEQRQKMSMGSSPSITIIGFEQRQKQPMSSSSPISNMVFEPRQKQPMSSSSPISNIVFEQRQLPTVGSSP
PISISGFEPKKQPSLSNSPPLSNLGFEQRLQPMSNASPISNLPFEQQRQQATMSNTRSAEPDSVESTTKWPLRMD
GAIGGCAGLPSSQKAPVIMQPETGTMKCPIPRTMPSNAKACPAVQNANSVNKRPLTVDDKDQTGSMNKKSMQKFL
GPQGCSRLESISALAHQKVSQSTTSGRALGPALNTNLKPRARQGSANDPQSIAARVRRERISERLKVLQALIPNG
DKVDMVTMLEKAISYVQCLEFQIKMLKNDSLWPKALGPLPNTLQELLELAGPEFAGIDGKNTEESSEKPKKSALE
VIELDGNQPSAD*
Physcomitrella RHD SIX LIKE 1 (PpRSL1) nucleotide sequence (SEQ ID NO: 27;
EF156393.1 GI: 140084326)
atggcaggtccagcaggagctttatggagtacttgtgatccacagcctattcaacaggcagagatatttagtggt
cctgacaaccaagctggtttgatgtcttttcatgtggataccccgttccattggggatctgaaccatgggctctc
cactctcggtcagatgacatcgccttgatgtccccctcgcttgttcacgacatatcaccttatgattctgtcttg
catctttccggagtgtctggggatgtgcaagatttagtttgcgggaatcccaaatttcgccaaagtgggcaatgg
gggcagagcgagttttcatactctgttcaggacaacatgcaagatctcctaaccaaccagttcataccgtacaac
acatcttcattgggtttaaatcatctctccccgaatttcaccgacttggattgcgcaccggtatacaatgatacc
aaggcttttggcactgttacacacaacagggcagtcccgagcactaatacccagagtgctcagcacgggagttcg
tctatggtttcaagtaacaggccaatcactagcacagcttctcctactactcagtatggaggtccgaggactcca
tcccaaaccacccagtacgggggttcatctatggttaccaactcgatggaaatgtttgcttcagctgcacctcag
ggtattatgactacatctggcttgagtggcggttgcaactcagacttgatgcatctgccgaagcgccagcatgct
cactctcttcctcctaccactggcagagatttaactgcatctgaagtggtatctggaaattcgatatcaaacatt
tccggggttggatcttttaacagcagccagaaaagcagtgcatccgtgatgatgtctcctttagctgcttcttct
cacatgcacaaggctgctgctgtatctgaagaacttaagatggcaagtttcaaccctggtccattcgtacctacg
cagaaaaagcagcaacatgagcagcaggatacgatgacctctaatcgtatatgggcggataagaacaacttggga
aaaattagttcatcgcccattccgatcatggggtttgagcagagtcaacagcaatccatgagcaattcctcccct
gttaccagtttggggtttgagcaaaggcaaaaaatgtccatgggtagctctccctccatcacgatcattggattt
gagcaaagacagaagcaacctatgagtagttcttcccccatttcaaacatggtttttgaaccaagacaaaaacag
ccaatgagtagctcttctcctatctctaatattgtctttgagcaaagacaactcccaactgtgggtagctctcct
ccgatttcaatctcaggatttgagccaaagaaacaaccatctttgagcaattctcctcccctctctaatctgggt
tttgagcaaaggctacaacccatgagtaatgcatctcctatttccaacttaccctttgagcaacaaagacaacaa
gcaaccatgagtaacaccagatctgcagaacccgattctgtcgagtctaccacgaagtggcccttgcggatggat
ggtgccataggtggatgtgctggcttaccaagcagtcagaaagctcctgttatcatgcagcctgagactgggact
atgaagtgtcctattccgaggaccatgcccagcaatgctaaggcttgcccagctgtgcagaatgctaattccgta
aacaagcgccctcttacggttgatgacaaggaccaaactggatcgatgaataagaagtcgatgcaaaagtttttg
ggacctcaaggttgtagcagacttgaaagtatcagtgctttagctcaccaaaaagtgagtcaaagtacaacaagc
ggtcgtgctctagggcctgctttgaacaccaatctcaagcctcgtgcacgccaagggagtgccaatgatccgcag
agcattgctgctagggtgcgaagagaaagaataagtgagcggctcaaagttttgcaagccttgatacctaacggt
gataaagtggatatggtcaccatgctggagaaggctatcagctacgtgcagtgtttggaatttcagattaagatg
ttaaaaaatgactctttgtggcctaaggcgcttggccctctaccgaacactttgcaagagcttctcgaacttgct
gggccagagtttgccggcatagatggcaagaatactgaggagtcgtcagagaaaccgaagaaatctgctcttgaa
gtaattgagttggacggcaatcagccttctgctgactaa
Physcomitrella RHD SIX LIKE 2 (PpRSL2) amino acid sequence (SEQ ID NO: 28
ABO84931.1 GI: 140084334)
MNKKPMQKALGPQGCSRLESISALAHQKVSQSASGRALGPALNTNLKPRARQGSANDPQSIAARVRRERISERLK
VLQALIPNGDKVDMVTMLEKAITYVQCLELQIKMLKNDSIWPKALGPLPNTLQELLELAGPEFSGTESKNVEEPP
AKPKKSAPDVIEFDGNQPSADKE*
Physcomitrella RHD SIX LIKE 2 (PpRSL2) nucleotide sequence (SEQ ID NO: 29;
EF156394.1 GI: 140084333)
atgaataagaagcctatgcaaaaagctttgggacctcaaggatgcagcaggctagaaagcatcagtgctttagct
catcaaaaagtgagtcagagtgcaagtggtcgtgcactagggcctgctctgaacaccaacctcaagcctcgtgct
cgtcaagggagtgccaatgacccacagagcattgccgctagggttcgaagagaaaggataagtgagcggctgaaa
gttttgcaagccttgatacctaatggtgataaggtagatatggtgaccatgctggagaaggctatcacctacgtg
cagtgtctggaactccagattaagatgttaaagaatgattctatctggcccaaggcgcttggacctctaccaaac
actcttcaagagcttctggagcttgctggaccagaattttctggaacggaaagcaagaatgtagaggagccccca
gcgaagccaaagaaatcagctcctgacgttattgagttcgacggcaatcaaccttctgccgacaaagagtag
Physcomitrella RHD SIX LIKE 3 (PpRSL3) amino acid sequence (SEQ ID NO: 30;
ABO84932.1 GI: 140084346)
GSATDPQSVYARHRREKINERLKTLQHLVPNGAKVDIVTMLDEAIHYVQFLQLQVTLLKSDEYWMYA
Physcomitrella RHD SIX LIKE 3 (PpRSL3) nucleotide sequence (SEQ ID NO: 31;
EF156395.1 GI:140084345)
ggttcagcgactgatccgcagagtgtatatgccaggcatagaagggagaagatcaacgagcgcttgaagacatta
cagcacttggtaccaaatggagctaaggtagacatcgtgaccatgcttgacgaagccattcactacgtccaattt
ctgcagctccaagtgacgctgttgaagtcggatgaatattggatgtacgcc
Physcomitrella RHD SIX LIKE 4 (PpRSL4) amino acid sequence (SEQ ID NO: 32;
ABO84933.1 GI:140084359)
MTDLISILESSGSSREEMCPVAVPSSVASSCERLIWEGWTAQPSPVEESTTSKLLPKLLPELETSSYSALTLQQP
DALSSILSVLHPFSHYSSASLELARNPDWSLKSSNPLRESSSEAGIRTSSFEGLYSGQHTTKKIHLGVIPYHLSE
DQRQCAVSPPENECRLLSANSSGSLHWWHSIGPESPSSTLAFHNIGIQHSTFEKCEPRGQSHSSWPAASGTSPTV
QYFHAHSADNEGVEVVKQDDSQISKALATYQPHGDHSLVLNSDRIASTTSHSEDPCGPKPGRRPAASYDTEMILS
PSESFLTTPNMLSTLECVISGASNISDQYMNFVREPQEQRLSSISDLSLIPDSHADPHSIGFISGTFRTDSHGTG
IRKNRIFLSDEESDFLPKKRSKYTVRGDFQMDRFDAVWGNTGLRGSSCPGNSVSQMMAIYEFGPALNRNGRPRVQ
RGSATDPQSVHARARREKIAERLRKLQHLIPNGGKVDIVTMLDEAVHYVQFLKRQVTLLKSDEYWMYATPTSYRS
KFDDCSLVPGENN*
Physcomitrella RHD SIX LIKE 4 (PpRSL4) nucleotide sequence (SEQ ID NO: 33;
EF156396.1 GI: 140084358)
ATGACCGATCTGATTTCGATCTTGGAGTCATCAGGGTCATCACGAGAGGAGATGTGCCCTGTTGCTGTGCCAAGC
TCCGTGGCTTCTTCTTGTGAAAGGTTGATATGGGAGGGGTGGACTGCACAACCATCTCCTGTCGAAGAAAGCACC
ACCAGCAAGTTACTTCCAAAGCTACTTCCAGAGCTCGAGACATCATCCTACTCTGCACTCACCCTTCAGCAACCT
GATGCGCTCTCCAGCATACTTTCAGTCCTCCACCCTTTTTCTCATTACAGTTCGGCCAGTTTAGAACTCGCTCGC
AATCCTGACTGGAGCTTGAAATCTTCAAATCCTCTGCGGGAAAGCAGCTCGGAGGCTGGCATCCGAACCTCATCT
TTCGAAGGCTTGTACTCTGGTCAGCACACCACCAAAAAGATTCATTTGGGGGTCATACCCTACCACTTGTCCGAA
GATCAGCGCCAGTGCGCTGTCAGTCCTCCGGAAAATGAGTGCCGCCTACTGTCTGCAAATTCCTCTGGATCCCTT
CACTGGTGGCATTCCATAGGCCCCGAGTCTCCTTCCTCTACTCTTGCATTCCATAATATTGGGATCCAACACTCT
ACCTTCGAAAAGTGTGAGCCTAGGGGCCAGTCGCACTCATCATGGCCAGCGGCCAGCGGCACGTCGCCAACAGTT
CAATACTTTCATGCCCATTCTGCAGATAATGAAGGTGTCGAGGTCGTCAAGCAAGATGACTCGCAGATATCCAAG
GCTCTGGCGACCTATCAACCCCACGGCGACCATAGTCTCGTGCTAAATTCAGACCGCATTGCAAGCACAACCAGC
CACTCAGAAGATCCTTGCGGCCCTAAACCTGGACGCAGACCAGCTGCATCATACGACACCGAGATGATTCTTAGC
CCAAGTGAGAGTTTCTTGACAACTCCCAATATGTTATCAACGTTGGAGTGCGTAATATCCGGTGCAAGTAACATA
TCTGATCAGTATATGAACTTCGTCAGAGAACCGCAGGAGCAAAGGCTGTCCTCTATCTCCGATCTGTCCCTTATT
CCTGACAGCCACGCGGATCCGCACAGTATCGGATTTATCTCTGGGACCTTTAGAACAGACTCCCACGGAACTGGA
ATAAGAAAGAACCGCATCTTTCTCAGTGATGAGGAATCCGACTTCTTGCCTAAGAAGCGATCCAAGTACACGGTC
CGCGGCGATTTTCAGATGGATCGCTTCGACGCAGTTTGGGGGAATACCGGTCTTCGGGGATCTAGCTGTCCTGGA
AATTCAGTATCCCAGATGATGGCGATTTACGAATTCGGACCCGCACTGAACAGGAACGGCAGGCCGCGAGTACAA
CGTGGTTCGGCGACTGATCCGCAGAGTGTACACGCCAGGGCGCGGAGGGAGAAAATCGCCGAGCGCTTGAGAAAG
TTGCAGCACCTCATTCCAAACGGCGGGAAGGTGGACATCGTAACCATGCTCGACGAAGCCGTTCACTATGTTCAG
TTTTTGAAGCGACAAGTTACGCTTCTGAAATCCGACGAGTATTGGATGTACGCCACGCCGACCTCGTACCGGAGC
AAATTCGACGACTGCAGTCTGGTTCCCGGCGAGAACAACTGA
Physcomitrella RHD SIX LIKE 5 (PpRSL5) amino acid sequence (SEQ ID NO: 34:
ABO84934.1 GI: 140084368)
MVQLYMSSVEEQRETMVQPYVSSMDSGSTSGRQTPSCVVQQGSNTFETSNLWEEWTQASNGDDTVSTSNFLPEIS
SFTSSRLSFQQSDSLTTWMSGFPPLSQTALSPDLSHSSDPVDHPPAFMQEGLGPGDSILDYSPALTEMYPKSSSK
HNSSDCLPYPAASAPDKKMTDHELGSAISLAYDRGTVSRQLLRALGPLSPSSPLALQNGLQNPLGDPWDASPSAM
PWPMATTGHAYGPGATRTSIPDHLANAINHLEGIAPSSASHASKPRHTDIFIAPNGTFDSTPGGWTPQYYDGSVT
TDESVKAMKLIASLREAGHAEATIGFCTESKPSFLRGGDRTTSPVDSFFGKCVGAKTSIKQACSGKHPLELEEIV
DSENSELNPTQLKRSKLFENHPNALWSDQSMNGRELRSYSHLVGSSLTASQPMDIIAIGPALNTDGKPRAKRGSA
TDPQSVYARHRREKINERLKSLQNLVPNGAKVDIVTMLDEAIHYVKFLQNQVELLKSDELWIYATPNKYNGMDIS
DLSDMYLQELESRA*
Physcomitrella RHD SIX LIKE 5 (PpRSL5) nucleotide sequence (SEQ ID NO: 35;
EF156397.1 GI: 140084367)
ATGGTGCAGTTATACATGTCCTCAGTTGAAGAGCAGCGGGAAACAATGGTACAGCCATACGTCTCAAGCATGGAC
TCAGGCTCAACGTCGGGGCGCCAGACGCCATCTTGCGTCGTTCAGCAGGGAAGTAACACATTTGAGACTTCGAAT
CTGTGGGAGGAATGGACGCAAGCATCGAACGGCGACGATACAGTCTCCACCAGCAATTTCCTCCCCGAAATCAGT
TCCTTCACGTCGAGTCGTCTCTCCTTCCAGCAAAGCGACTCTCTCACCACTTGGATGTCAGGGTTCCCTCCCCTC
TCCCAAACTGCCTTGAGCCCGGATCTTAGTCACTCCTCCGACCCCGTGGATCATCCCCCAGCATTCATGCAGGAG
GGTTTAGGCCCCGGTGATTCTATTCTGGACTATTCCCCCGCTCTCACAGAGATGTACCCGAAAAGTAGCTCCAAA
CATAATTCCTCGGATTGTTTACCTTACCCTGCGGCCAGTGCACCAGACAAAAAAATGACTGATCACGAACTAGGT
TCGGCTATTTCCCTCGCGTATGATAGAGGCACCGTTTCCCGCCAGCTTCTTCGAGCCTTGGGCCCATTGTCGCCT
TCATCGCCTCTAGCATTGCAGAATGGGCTGCAAAACCCGCTTGGGGACCCCTGGGATGCTTCTCCATCTGCAATG
CCGTGGCCAATGGCAACAACCGGTCATGCTTATGGACCAGGCGCCACCAGGACTTCTATTCCAGATCACTTAGCA
AATGCAATTAATCACCTGGAGGGCATTGCACCGTCCAGTGCCAGTCATGCATCGAAACCTCGTCACACTGATATT
TTCATTGCACCCAATGGCACGTTCGATTCGACGCCGGGAGGTTGGACACCGCAGTATTACGATGGGTCCGTGACG
ACAGATGAGTCTGTGAAGGCGATGAAGCTGATTGCGTCCCTACGTGAAGCAGGCCACGCAGAGGCTACAATTGGA
TTCTGTACAGAGAGCAAGCCTAGTTTTCTCAGGGGTGGGGACAGAACAACCTCGCCAGTGGACAGCTTCTTCGGC
AAATGTGTAGGGGCCAAAACGAGTATAAAGCAAGCCTGTTCTGGGAAACACCCTCTTGAACTTGAGGAGATCGTT
GATAGTGAAAACAGTGAATTAAATCCCACCCAGCTCAAACGCTCTAAACTTTTTGAGAATCATCCGAATGCCTTG
TGGAGCGATCAGAGTATGAATGGAAGAGAACTGAGATCGTACTCTCATTTGGTTGGCAGCAGTCTTACTGCATCG
CAGCCCATGGACATAATTGCAATTGGCCCAGCGCTCAACACTGATGGCAAACCACGAGCAAAGCGGGGTTCAGCA
ACCGATCCTCAGAGTGTTTACGCTAGACATAGGAGAGAAAAAATCAACGAACGATTGAAGAGTTTACAAAACCTA
GTACCTAATGGAGCCAAGGTTGACATAGTAACCATGCTGGACGAAGCTATACATTACGTCAAATTTTTACAAAAT
CAAGTTGAGCTGCTGAAGTCCGACGAGTTGTGGATTTACGCAACACCAAATAAGTACAACGGCATGGACATTTCC
GACCTCTCTGACATGTATTTGCAGGAGCTGGAGTCACGTGCGTGA
Physcomitrella RHD SIX LIKE 6 (PpRSL6) amino acid sequence (SEQ ID NO: 36;
ABO84935.1 GI: 140084376)
MVRFNYMYPVQEQLEAMTDQHTPSMDSVSSAGEKTSSCIVQQGGNASETSNLWEEWTQGSNGDDSVSTSNFLPEL
NSSTSSRLAFHQSDILSTWISGYHPLSQSSLSSEFSHTSDRENHPPAFMQEGLIPSGLILDSDPALTDIYTRSSS
SDSLPYPTARIMDKALTDHELESAVPLAYEKGCVPPQVLRNLGPLSPSSPLAFQNGLLNPLRDPWDSCPSALPWS
NVTTASQTYGQVTTRTFIPDHSASAIDKLEAVATITAGYGASKPQHTDVFIEPNGTFQSTPAGWAPQFYDGSEAT
GLLVKPMRAIASLGEAGCGEATSEFCTKTKPGLLKGGDTITSPVGSLLGDCKKAESSMKQVWPGKHRLELVELVD
GEDTKSSPTQLKRPKHSTDYANVLLSDHILKGAELRSYFHSGDVGLNASQAMDIIVIGPALNTNGKPRAKRGSAT
DPQSVYARHRREKINERLKNLQNLVPNGAKVDIVTMLDEAIHYVKFLQTQVELLKSDEFWMFANPHNYNGIDISD
PSSMHSPELESNI*
Physcomitrella RHD SIX LIKE 6 (PpRSL6) nucleotide sequence (SEQ ID NO: 37:
EF156398.1 GI: 140084375)
ATGGTGCGGTTTAACTACATGTACCCGGTTCAAGAGCAGCTGGAAGCCATGACGGACCAACACACCCCAAGCATG
GATTCGGTCTCGTCGGCCGGAGAGAAGACATCCTCTTGCATCGTCCAGCAGGGAGGAAATGCATCCGAAACTTCA
AACTTGTGGGAAGAATGGACACAAGGGTCGAACGGCGACGATTCTGTCTCTACCAGCAACTTCCTCCCCGAACTG
AATTCCTCCACCTCCAGTCGTCTCGCATTCCACCAAAGCGACATTCTTTCCACTTGGATCTCAGGCTACCACCCA
CTCTCGCAAAGCAGCCTGAGTTCCGAATTCAGCCACACCTCCGACCGCGAGAATCACCCCCCAGCATTCATGCAA
GAGGGTTTAATCCCCAGTGGTTTAATTCTTGACTCTGATCCTGCTCTCACAGATATTTATACGAGAAGCAGCTCC
TCGGACTCTTTGCCATACCCCACGGCTAGGATCATGGACAAAGCATTGACCGATCACGAGCTTGAGTCTGCTGTC
CCACTTGCATATGAAAAAGGCTGCGTTCCTCCCCAGGTTCTGCGTAACCTAGGGCCATTGTCACCTTCTTCGCCT
CTGGCATTCCAGAATGGACTGCTAAACCCCCTCAGGGACCCTTGGGATTCGTGTCCATCTGCATTGCCATGGTCA
AATGTGACCACAGCCAGCCAGACTTACGGTCAAGTGACAACCAGGACTTTCATTCCAGATCACTCTGCAAGTGCA
ATCGACAAGTTGGAGGCCGTCGCAACGATCACTGCCGGATACGGCGCGTCGAAACCACAACATACTGACGTCTTC
ATAGAACCCAACGGGACGTTTCAGTCGACTCCGGCAGGGTGGGCACCGCAGTTTTACGATGGATCCGAGGCGACG
GGCCTGTTGGTCAAGCCAATGAGGGCCATCGCATCTCTGGGTGAAGCCGGCTGTGGGGAGGCCACTAGTGAATTC
TGCACAAAGACCAAGCCAGGACTTCTCAAAGGTGGGGACACAATAACCTCGCCGGTGGGTAGCCTGTTGGGCGAT
TGCAAAAAAGCTGAGTCAAGTATGAAGCAAGTTTGGCCTGGAAAACACCGTCTTGAACTCGTGGAACTAGTCGAT
GGTGAAGACACCAAATCAAGTCCCACCCAGCTCAAACGGCCGAAACATTCTACGGATTATGCGAATGTCCTGTTG
AGCGATCATATTCTGAAAGGAGCGGAGCTGCGGTCCTACTTCCATTCTGGTGATGTTGGTCTAAATGCATCTCAA
GCGATGGACATTATTGTAATTGGCCCAGCCTTGAATACTAATGGCAAGCCGCGAGCTAAACGGGGTTCAGCCACC
GATCCCCAGAGTGTGTACGCTAGACATAGGCGAGAAAAAATCAACGAACGACTGAAGAATTTACAAAATCTCGTG
CCAAATGGAGCCAAGGTTGACATTGTGACCATGCTAGACGAAGCCATACACTACGTCAAATTCTTGCAAACTCAA
GTTGAGCTGCTGAAATCCGACGAGTTCTGGATGTTCGCAAATCCACACAACTACAACGGCATAGATATCTCCGAT
CCCTCTAGCATGCATTCGCCGGAGCTGGAGTCGAATATTTAG
Physcomitrella RHD SIX LIKE 7 (PpRSL7) amino acid sequence (SEQ ID NO: 38;
ABO84936.1 GI: 140084384)
GSATDPQSVYARHRREKINERLKTLQRLVPNGEQVDIVTMLEEAIHFVKFLEFQLELLRSDDRWMFA
Physcomitrella RHD SIX LIKE 7 (PpRSL7) nucleotide sequence (SEQ ID NO: 39;
EF156399.1 GI: 140084383)
Gggtcagctactgatcctcagagtgtgtacgcaaggcatcgccgggagaagattaacgagcgcctaaagacattg
cagcggttggttcctaacggagaacaggtcgacattgtgaccatgctggaagaagccattcactttgtcaaattt
ttggagttccaactggagctgttgcgatccgatgatcgctggatgttcgcc
Selaginella moelendorfii SmRSLa amino acid sequence (SEQ ID NO: 40)
LNTNLKPRAKQGCANDPQSIAARQRRERISDRLKILQELIPNGSKVDLVTMLEKAINYVKFLQLQVKVLMNDEYW
PPKGD
Selaginella moelendorfii SmRSLa nucleotide sequence (SEQ ID NO: 41)
CTCAACACTAATCTTAAGCCGCGAGCAAAGCAAGGTTGTGCTAATGATCCACAAAGCATTGCTGCCAGACAACGA
AGAGAACGGATAAGTGACCGGCTTAAAATCCTGCAGGAGCTCATACCAAATGGATCCAAGGTCGATCTGGTAACC
ATGCTGGAGAAGGCCATCAACTACGTCAAGTTCTTGCAATTGCAAGTCAAAGTTCTTATGAACGATGAGTATTGG
CCACCAAAGGGAGAT
Selaginella moelendorfii SmRSLb amino acid sequence (SEQ ID NO: 42)
LNTNLKPRAKQGCANDPQSIAARQRRERISDRLKILQELIPNGSKVDLVTMLEKAINYVKFLQLQVKVLMNDEYW
PPKGD
Selaginella moelendorfii SmRSLb nucleotide sequence (SEQ ID NO: 43)
CTCAACACTAATCTTAAGCCGCGAGCAAAGCAAGGTTGTGCTAATGATCCACAAAGCATTGCTGCCAGACAACGA
AGAGAACGGATAAGTGACCGGCTTAAAATCCTGCAGGAGCTCATACCAAATGGATCCAAGGTCGATCTGGTAACC
ATGTTGGAGAAGGCCATCAACTACGTCAAGTTCTTGCAATTGCAAGTCAAAGTTCTTATGAACGATGAGTATTGG
CCACCAAAGGGAGAT
Selaginella moelendorfii SmRSLc amino acid sequence (SEQ ID NO: 44)
LNTNFKPRARQGSANDPQSIAARHRRERISDRLKILQELVPNSTKVDLVTMLEKAINYVKFLQLQVKVLTSDDYW
P
Selaginella moelendorfii SmRSLc nucleotide sequence (SEQ ID NO: 45)
CTCAACACCAATTTCAAGCCTCGAGCCAGGCAGGGAAGCGCCAATGATCCCCAGAGCATCGCTGCTAGACATCGC
CGGGAGAGGATCAGTGACAGGCTCAAGATCTTGCAAGAGCTCGTTCCAAACAGCACAAAGGTTGATCTAGTGACG
ATGCTGGAGAAGGCCATCAATTACGTCAAGTTCCTCCAGCTGCAAGTTAAGGTGCTTACGTCGGACGACTACTGG
CCA
Selaginella moelendorfii SmRSLd amino acid sequence (SEQ ID NO: 46)
LNTNFKPRARQGSANDPQSIAARHRRERISDRLKILQELVPNSTKVDLVTMLEKAINYVKFLQLQVKVLTSDDYW
P
Selaginella moelendorfii SmRSLd nucleotide sequence (SEQ ID NO: 47)
CTCAACACCAATTTCAAGCCTCGAGCCAGGCAGGGAAGCGCCAATGATCCCCAGAGCATCGCTGCTAGACATCGC
CGGGAGAGGATCAGTGACAGGCTCAAGATCTTGCAAGAGCTCGTTCCAAACAGCACAAAGGTTGATCTAGTGACG
ATGCTGGAGAAGGCCATCAATTACGTCAAGTTCCTCCAGCTGCAAGTTAAGGTGCTTACGTCGGACGACTATTGG
CCA
Selaginella moelendorfii SmRSLe amino acid sequence (SEQ ID NO: 48)
LNTDGKPRAKRGSATDPQSIYARQRRERINERLRALQGLVPNGAKVDIVTMLEEAINYVKFLQLQVKLLSSDEYW
MYAPT
Selaginella moelendorfii SmRSLe nucleotide sequence (SEQ ID NO: 49)
CTAAACACCGACGGAAAGCCACGCGCAAAGCGTGGATCTGCCACGGACCCGCAAAGCATCTACGCTCGGCAAAGA
AGAGAAAGGATCAACGAGCGTTTGAGAGCGCTACAAGGACTCGTACCAAACGGAGCGAAGGTTGACATTGTGACG
ATGCTCGAGGAAGCCATCAACTATGTCAAGTTTTTGCAGCTGCAAGTAAAGCTGCTCAGCTCGGACGAGTATTGG
ATGTACGCCCCCACA
Selaginella moelendorfii SmRSLf amino acid sequence (SEQ ID NO: 50)
LNTNGKPRAKRGSATDPQSVYARHRRERINERLKTLQHLVPNGAKVDIVTMLERAIHYVKFLQLQVNMLSSDEYW
IYAPT
Selaginella moelendorfii SmRSLf nucleotide sequence (SEQ ID NO: 51)
CTCAACACGAATGGCAAGCCCAGAGCAAAGCGTGGATCTGCAACAGATCCCCAAAGCGTTTACGCAAGGCACCGG
AGAGAGAGGATCAACGAGAGGCTCAAAACTTTACAACACCTTGTTCCAAATGGTGCAAAGGTTGACATAGTGACA
ATGCTTGAAGAAGCAATACATTACGTGAAGTTTCTACAGCTGCAAGTCAACATGTTAAGCTCTGATGAGTACTGG
ATTTATGCACCCACA
Selaginella moelendorfii SmRSLg amino acid sequence (SEQ ID NO: 52)
LNTNGKPRAKRGSATDPQSVYARHRRERINERLKTLQHLVPNGAKVDIVTMLEEAIHYVKFLQLQVNMLSSDEYW
TYAPT
Selaginella moelendorfii SmRSLg nucleotide sequence (SEQ ID NO: 53)
CTCAACACGAATGGCAAGCCCCGAGCAAAGCGTGGATCTGCAACAGATCCCCAAAGCGTTTATGCAAGGCACCGG
AGAGAGAGGATCAACGAGAGGCTCAAAACTTTACAACACCTTGTTCCAAATGGTGCAAAGGTTGACATTGTGACA
ATGCTTGAAGAAGCAATACATTACGTGAAGTTTCTACAGCTGCAAGTCAACATGTTAAGCTCTGATGAGTACTGG
ACTTATGCACCCACA
Selaginella moelendorfii SmRSLh amino acid sequence (SEQ ID NO: 54)
LNTDGKPRAKRGSATDPQSIYARQRRERINERLRALQGLVPNGAKVDIVTMLEEAINYVKFLQLQVKLLSSDEYW
MYAPT
Selaginella moelendorfii SmRSLh nucleotide sequence (SEQ ID NO: 55)
CTAAACACCGACGGAAAGCCACGCGCAAAGCGTGGATCTGCCACGGACCCGCAAAGTATCTACGCTCGGCAAAGA
AGAGAAAGGATCAACGAGCGTTTGAGAGCGCTACAAGGACTCGTACCAAACGGAGCGAAGGTTGACATTGTGACG
ATGCTCGAGGAAGCCATCAACTATGTCAAGTTTTTGCAGCTGCAAGTAAAGCTGCTCAGCTCGGACGAGTATTGG
ATGTACGCCCCCACA
Rice (Oryza sativa subsp. Japonica) OsRSLa amino acid sequence
(SEQ ID NO: 56; LOC_Os01g02110.1 11971.m06853)
MMAAQASSKRGMLLPREAVLYDDEPSMPLEILGYHGNGVGGGGCVDADYYYSWSGSSSSSSSSVLSFDQAAVGGS
GGGCARQLAFHPGGDDDDCAMWMDAAAGAMVENTSVVAGGGNNYCHRLQFHGGAAGFGLASPGSSVVDNGLEIHE
SNVSKPPPPAAKKRACPSGEARAAGKKQCRKGSKPNKAASASSPSPSPSPSPSPNKEQPQSAAAKVRRERISERL
KVLQDLVPNGTKVDLVTMLEKAINYVKFLQLQVKVLATDEFWPAQGGKAPELSQVKDALDAILSSQHPNK*
Rice OsRSLa nucleotide sequence (SEQ ID NO: 57; LOC_Os01g02110.1 11971.m06853)
ATGATGGCAGCTCAGGCAAGCAGCAAGCGCGGCATGCTGCTGCCACGGGAGGCGGTGCTCTACGACGACGAGCCC
TCCATGCCGCTGGAGATCTTGGGCTACCACGGCAATGGCGTCGGCGGCGGTGGCTGCGTTGACGCCGATTACTAC
TACAGCTGGTCGGGGTCCAGCTCCAGCTCCAGCTCGTCGGTGCTCAGCTTTGACCAGGCGGCGGTCGGCGGCAGC
GGCGGCGGCTGCGCCCGGCAGCTGGCTTTCCATCCCGGCGGCGACGACGACGACTGCGCCATGTGGATGGACGCC
GCCGCCGGCGCCATGGTCGAGAACACGTCTGTCGTCGCCGGCGGCGGCAACAACTACTGTCATCGCCTGCAGTTC
CACGGCGGCGCCGCCGGTTTCGGACTCGCGAGCCCAGGCTCGTCGGTCGTTGACAACGGCCTCGAAATCCACGAG
AGCAACGTCAGCAAGCCGCCACCGCCGGCAGCCAAGAAGCGCGCATGCCCGAGCGGCGAGGCGAGAGCAGCGGGG
AAGAAGCAGTGCAGGAAAGGGAGCAAGCCAAACAAGGCTGCTTCTGCTTCTTCTCCTTCTCCTTCTCCTTCTCCT
TCTCCTTCTCCTAACAAGGAACAACCTCAAAGCGCCGCTGCAAAGGTAAGAAGAGAGCGGATCAGTGAGAGGCTC
AAAGTTCTTCAGGATCTCGTGCCTAATGGCACAAAGGTAGACTTGGTCACCATGCTAGAAAAGGCGATCAACTAC
GTCAAATTCCTCCAGCTGCAAGTGAAGGTTTTGGCTACTGATGAGTTCTGGCCGGCACAAGGAGGGAAAGCACCA
GAGCTCTCTCAAGTCAAGGACGCCTTGGACGCCATCCTATCTTCTCAGCATCCAAACAAATGA
Rice OsRSLb amino acid sequence (SEQ ID NO: 58; LOC_Os02g48060.1 11972.m09840)
MRMALVRERAMVYGGGCDAEAFGGGFESSQMGYGHDALLDIDAAALFGGYEAAASAGCALVQDGAAGWAGAGASS
SVLAFDRAAQAEEAECDAWIEAMDQSYGAGGEAAPYRSTTAVAFDAATGCFSLTERATGGGGGAGGRQFGLLFPS
TSGGGVSPERAAPAPAPRGSQKRAHAESSQAMSPSKKQCGAGRKAGKAKSAPTTPTKDPQSLAAKNRRERISERL
RILQELVPNGTKVDLVTMLEKAISYVKFLQLQVKVLATDEFWPAQGGKAPEISQVKEALDAILSSSSPLMGQLMN*
Rice OsRSLb nucleotide sequence (SEQ ID NO: 59; LOC_Os02g48060.1 11972.m09840)
ATGCGCATGGCGCTGGTGCGGGAGCGCGCGATGGTGTACGGTGGAGGGTGCGACGCCGAGGCGTTCGGCGGCGGG
TTCGAGTCGTCCCAGATGGGGTACGGCCACGACGCGCTGCTCGACATCGACGCGGCGGCGCTGTTCGGGGGGTAC
GAGGCGGCCGCCAGCGCCGGGTGCGCCCTCGTGCAGGACGGCGCCGCGGGGTGGGCGGGCGCGGGCGCGTCGTCC
TCGGTGCTGGCGTTCGACCGCGCCGCTCAGGCGGAGGAGGCCGAGTGCGACGCGTGGATCGAAGCCATGGACCAG
AGCTACGGCGCCGGCGGCGAGGCGGCGCCGTACCGGTCGACGACGGCCGTCGCCTTCGACGCGGCCACCGGCTGC
TTCAGCCTGACGGAGAGAGCCACCGGCGGCGGCGGCGGCGCGGGTGGGCGGCAGTTCGGGCTGCTGTTCCCGAGC
ACGTCGGGCGGCGGCGTCTCCCCCGAACGCGCCGCGCCGGCGCCGGCGCCCCGCGGCTCGCAGAAGCGGGCCCAC
GCGGAGTCGTCGCAGGCCATGAGCCCTAGCAAGAAGCAGTGCGGCGCCGGCAGGAAGGCGGGCAAGGCCAAGTCG
GCGCCGACCACCCCAACCAAGGACCCGCAAAGCCTCGCGGCCAAGAATCGGCGCGAGAGGATCAGCGAGCGGCTG
CGGATCCTGCAGGAGCTCGTGCCCAACGGCACCAAGGTCGACCTCGTCACCATGCTCGAGAAGGCCATCAGCTAC
GTCAAGTTCCTCCAGCTTCAAGTCAAGGTTCTTGCGACGGACGAGTTCTGGCCGGCGCAGGGAGGGAAGGCGCCG
GAGATATCCCAGGTGAAGGAGGCGCTCGACGCCATCTTGTCGTCGTCGTCGCCGCTGATGGGACAACTCATGAAC
TGA
Rice OsRSLc amino acid sequence (SEQ ID NO: 60; LOC_Os06g30090.1 11976.m07553)
MAMVAGDEAMSVPWHDVGVVVDPEAAGTAPFDAGAGYVPSYGQCQYYYYYDDHHHHPCSTELIHAGDAGSAVAVA
YDGVDGWVHAAAAATSPSSSSALTFDGHGAEEHSAVSWMDMDMDAHGAAPPLIGYGPTAATSSPSSCFSSGGSGD
SGMVMVTTTTPRSAAASGSQRRARPPPSPLQGSELHEYSKKQRANNKETQSSAAKSRRERISERLRALQELVPSG
GKVDMVTMLDRAISYVKFMQMQLRVLETDAFWPASDGATPDISRVKDALDAIILSSSSPSQKASPPRSG*
Rice OsRSLc nucleotide sequence (SEQ ID NO: 61; LOC_Os06g30090.1 11976.m07553)
ATGGCTATGGTGGCCGGCGACGAGGCGATGTCAGTGCCATGGCACGACGTCGGCGTCGTCGTCGACCCCGAGGCG
GCCGGGACGGCGCCGTTCGACGCCGGCGCCGGCTATGTCCCATCGTACGGTCAGTGCCAATACTACTACTACTAC
GACGACCACCACCACCACCCGTGCAGCACGGAGCTGATCCACGCGGGCGACGCTGGCAGTGCGGTTGCGGTTGCG
TACGACGGCGTCGACGGCTGGGTTCACGCCGCCGCCGCAGCCACCTCCCCGTCCTCGTCATCTGCGCTCACCTTC
GATGGTCACGGCGCCGAGGAGCACAGCGCAGTGTCGTGGATGGACATGGACATGGACGCGCACGGCGCCGCGCCT
CCCCTAATCGGCTACGGCCCGACGGCGGCGACCTCCTCCCCCTCCTCCTGCTTCAGCTCCGGCGGCTCCGGCGAC
AGCGGCATGGTGATGGTGACCACCACCACCCCGAGGAGCGCCGCCGCCTCTGGTTCGCAGAGGCGGGCACGCCCG
CCGCCGTCGCCGTTGCAGGGATCAGAGCTGCACGAGTACTCCAAGAAGCAGCGCGCCAACAACAAGGAGACACAG
AGCTCAGCTGCCAAGAGCCGGCGGGAGAGGATCAGCGAGCGGCTGAGGGCGCTGCAGGAGCTGGTGCCGAGCGGC
GGGAAGGTGGACATGGTGACCATGCTGGACAGGGCCATCAGCTACGTCAAGTTCATGCAGATGCAGCTCAGGGTG
CTGGAGACCGACGCGTTCTGGCCGGCGTCCGACGGCGCCACGCCGGACATCTCCCGGGTCAAGGACGCGCTCGAC
GCCATCATCCTCTCCTCGTCCTCGCCCTCGCAAAAGGCTTCTCCTCCTCGGTCGGGCTAG
Rice OsRSLd amino acid sequence (SEQ ID NO: 62; LOC_Os03g10770.1 11973.m06529)
MEDSEAMAQLLGVQYFGNDQEQQQPAAAAPPAMYWPAHDAADQYYGSAPYCYMQQQQHYGCYDGGAMVAGGDFFV
PEEQLVADPSFMVDLNLEFEDQHGGDAGGAGSSAAAAAAATKMTPACKRKVEDHKDESCTDNVARKKARSTAATV
VQKKGNKNAQSKKAQKGACSRSSNQKESNGGGDGGNVQSSSTNYLSDDDSLSLEMTSCSNVSSASKKSSLSSPAT
GHGGAKARAGRGAATDPQSLYARKRRERINERLKILQNLIPNGTKVDISTMLEEAVHYVKFLQLQIKLLSSDDMW
MFAPIAYNGVNVGLDLKISPPQQQ*
Rice OsRSLd nucleotide sequence (SEQ ID NO: 63; LOC_Os03g10770.1 11973.m06529)
ATGGAGGACTCGGAGGCGATGGCGCAGCTGCTCGGCGTGCAGTACTTCGGCAATGACCAGGAGCAGCAGCAGCCG
GCGGCGGCGGCGCCGCCGGCGATGTACTGGCCGGCGCACGACGCGGCCGACCAGTACTACGGCTCGGCGCCATAC
TGCTACATGCAGCAGCAGCAGCATTACGGGTGCTACGACGGCGGCGCGATGGTGGCCGGCGGCGACTTCTTCGTG
CCGGAGGAGCAGCTGGTGGCCGACCCGAGCTTCATGGTGGACCTGAACCTCGAGTTCGAGGACCAGCACGGCGGC
GATGCTGGCGGCGCTGGGAGCAGCGCCGCCGCCGCCGCCGCCGCCACCAAGATGACACCGGCGTGCAAGAGGAAG
GTTGAGGATCACAAGGATGAGAGCTGCACGGACAACGTCGCGAGGAAGAAGGCGCGCTCCACGGCAGCAACAGTG
GTGCAGAAGAAGGGTAATAAGAACGCGCAGTCAAAGAAGGCGCAGAAGGGCGCGTGCAGCCGGAGCAGCAACCAG
AAGGAGAGCAATGGCGGCGGCGACGGCGGCAATGTGCAGAGCTCGAGCACCAACTACCTCTCTGATGACGACTCG
CTGTCGCTGGAGATGACTTCGTGCAGCAACGTGAGCTCGGCGTCCAAGAAGTCGTCGTTGTCATCGCCGGCGACC
GGGCACGGCGGCGCGAAGGCGAGGGCCGGGCGCGGGGCGGCGACCGATCCGCAAAGCCTCTATGCCAGGAAGAGG
AGAGAAAGGATCAATGAACGGCTAAAGATACTGCAGAATCTTATCCCAAATGGAACCAAGGTGGACATCAGCACG
ATGCTTGAAGAAGCAGTTCACTACGTCAAGTTCTTGCAGCTCCAAATCAAGCTTCTGAGCTCGGATGATATGTGG
ATGTTCGCGCCGATCGCGTACAACGGGGTCAACGTCGGGCTCGACCTCAAGATCTCTCCACCGCAGCAGCAATGA
Rice OsRSLe amino acid sequence (SEQ ID NO: 64; LOC_Os03g42100.1 11973.m09268)
MESGGVIAEAGWSSLDMSSQAEESEMMAQLLGTCFPSNGEDDHHQELPWSVDTPSAYYLHCNGGSSSAYSSTTSS
NSASGSFTLIAPRSEYEGYYVSDSNEAALGISIQEQGAAQFMDAILNRNGDPGFDDLADSSVNLLDSIGASNKRK
IQEQGRLDDQTKSRKSAKKAGSKRGKKAAQCEGEDGSIAVTNRQSLSCCTSENDSIGSQESPVAAKSNGKAQSGH
RSATDPQSLYARKRRERINERLKILQNLVPNGTKVDISTMLEEAMHYVKFLQLQIKLLSSDEMWMYAPIAYNGMN
IGIDLNLSQH*
Rice OsRSLe nucleotide sequence (SEQ ID NO: 65; LOC_Os03g42100.1 11973.m09268)
ATGGAGTCCGGAGGGGTGATCGCGGAGGCGGGGTGGAGCTCGCTCGACATGTCGTCGCAGGCCGAGGAGTCGGAG
ATGATGGCGCAGCTGCTTGGAACCTGCTTCCCCTCCAATGGCGAGGATGATCATCACCAAGAGCTTCCTTGGTCG
GTTGACACCCCCAGTGCCTACTACCTCCATTGCAATGGAGGTAGCTCAAGTGCATACAGCTCTACCACTAGCAGC
AACAGTGCTAGTGGTAGCTTCACTCTCATTGCACCAAGATCTGAGTATGAGGGGTACTATGTGAGTGACTCTAAT
GAGGCGGCCCTCGGGATCAGCATCCAGGAGCAAGGTGCAGCTCAGTTCATGGATGCCATTCTCAACCGGAACGGC
GATCCGGGCTTCGATGATCTCGCTGACTCGAGCGTTAATCTGCTGGATTCCATCGGCGCTTCTAACAAGAGAAAG
ATTCAGGAGCAAGGCAGGCTAGATGACCAAACGAAAAGTAGGAAATCTGCGAAGAAGGCTGGCTCGAAGCGGGGA
AAGAAGGCGGCGCAATGTGAAGGTGAAGATGGCAGCATTGCTGTCACCAACAGGCAAAGCTTGAGCTGCTGCACC
TCTGAAAATGATTCGATTGGTTCTCAAGAATCTCCTGTTGCTGCTAAGTCGAATGGCAAGGCTCAATCTGGCCAT
CGGTCAGCAACCGATCCCCAGAGCCTCTATGCAAGGAAAAGAAGAGAGAGGATCAATGAGAGGCTCAAGATTCTG
CAGAACCTTGTACCAAATGGAACCAAAGTAGATATCAGCACTATGCTTGAAGAGGCAATGCATTACGTGAAGTTC
TTGCAGCTTCAAATCAAGCTCCTCAGCTCTGATGAAATGTGGATGTACGCACCGATTGCTTACAACGGGATGAAC
ATCGGGATCGATTTGAACCTCTCTCAGCATTGA
Rice OsRSLf amino acid sequence (SEQ ID NO: 66; LOC_Os11g41640.1 11981.m08005)
MDARCANIWSSADARSEESEMIDQLKSMFWSSTDAEINFYSPDSSVNSCVTTSTMPSSLFLPLMDDEGFGTVQLM
HQVITGNKRMFPMDEHFEQQQKKPKKKTRTSRSVSSSSTITDYETSSELVNPSCSSGSSVGEDSIAATDGSVVLK
QSDNSRGHKQCSKDTQSLYAKRRRERINERLRILQQLVPNGTKVDISTMLEEAVQYVKFLQLQIKLLSSDDTWMF
APLAYNGMNMDLGHTLAENQE*
Rice OsRSLf nucleotide sequence (SEQ ID NO: 67; LOC_Os11g41640.1 11981.m08005)
ATGGATGCAAGGTGTGCAAACATCTGGAGCTCTGCTGATGCAAGGAGTGAGGAATCTGAGATGATTGATCAACTA
AAGTCCATGTTCTGGAGCAGCACTGATGCTGAAATCAACTTTTATTCTCCTGACAGTAGTGTAAATTCTTGTGTC
ACAACTAGCACAATGCCTAGCAGCTTGTTTCTTCCTCTGATGGATGATGAGGGATTTGGCACAGTGCAATTGATG
CATCAGGTCATCACTGGGAACAAGAGGATGTTCCCCATGGATGAGCACTTTGAGCAGCAGCAGAAGAAGCCGAAG
AAGAAAACCCGAACTTCTCGCTCGGTATCAAGTAGTTCAACCATTACTGACTATGAGACTAGCTCTGAACTTGTC
AATCCTAGCTGTTCCTCCGGGAGCAGCGTCGGAGAGGATTCAATTGCTGCAACTGATGGATCTGTAGTGCTGAAA
CAAAGTGACAATTCAAGAGGCCATAAGCAGTGCTCCAAGGATACACAAAGCCTCTATGCTAAGAGGAGAAGGGAA
AGGATTAATGAGAGACTGAGAATACTTCAGCAGCTTGTTCCCAATGGCACTAAAGTTGACATCAGCACAATGCTG
GAGGAAGCAGTTCAGTATGTCAAGTTTTTGCAGTTGCAAATAAAGCTATTGAGCTCTGACGACACATGGATGTTT
GCGCCCCTAGCCTATAATGGCATGAACATGGATCTCGGTCATACTCTTGCTGAAAACCAAGAATGA
Rice OsRSLg amino acid sequence (SEQ ID NO: 68; LOC_Os12g32400.1 11982.m07043)
MECSSFEAICNESEMIAHLQSLFWSSSDADPCFGSSSFSLISSEGYDTMTTEFVNSSTNVCFDYQDDSFVSAEET
TIGNKRKVQMDTENELMTNRSKEVRTKMSVSKACKHSVSAESSQSYYAKNRRQRINERLRILQELIPNGTKVDIS
TMLEEAIQYVKFLHLQIKLLSSDEMWMYAPLAFDSGNNRLYQNSLSQE*
Rice OsRSLg nucleotide sequence (SEQ ID NO: 69; LOC_Os12g32400.1 11982.m07043)
ATGGAATGCAGCTCCTTTGAAGCAATCTGCAATGAGTCGGAGATGATTGCGCATTTGCAGTCATTGTTCTGGAGC
AGCAGCGATGCTGATCCTTGTTTTGGTAGCTCATCATTTTCTCTCATCAGTAGTGAGGGCTACGACACAATGACC
ACAGAGTTTGTGAATAGCAGCACAAATGTATGTTTTGATTACCAAGATGATAGCTTCGTTTCAGCAGAGGAGACT
ACCATTGGTAACAAGAGAAAAGTTCAGATGGATACTGAGAATGAGCTGATGACGAACCGCAGCAAGGAAGTTCGC
ACCAAGATGTCGGTGTCAAAAGCATGCAAACATTCTGTTTCTGCAGAGAGCTCACAGTCTTATTATGCAAAGAAC
AGGAGACAGAGGATCAATGAGAGATTGAGAATACTGCAAGAACTGATCCCTAATGGAACAAAAGTTGACATCAGC
ACAATGTTGGAGGAAGCAATTCAGTATGTCAAGTTTCTACACCTGCAAATCAAGCTCTTGAGCTCTGATGAAATG
TGGATGTATGCGCCCCTTGCTTTTGACAGTGGTAACAACAGGCTCTATCAGAACTCTCTGTCACAAGAGTAG
Rice OsRSLh amino acid sequence (SEQ ID NO: 70; LOC_Os12g39850.1 11982.m07769)
MEGGGLIADMSWTVFDLPSHSDESEMMAQLFSAFPIHGEEEGHEQLPWFDQSSNPCYYSCNASSTAYSNSNASSI
PAPSEYEGYCFSDSNEALGVSSSIAPHDLSMVQVQGATEFLNVIPNHSLDSFGNGELGHEDLDSVSGTNKRKQSA
EGEFDGQTRGSKCARKAEPKRAKKAKQTVEKDASVAIPNGSCSISDNDSSSSQEVADAGATSKGKSRAGRGAATD
PQSLYARKRRERINERLKTLQNLVPNGTKVDISTMLEEAVHYVKFLQLQIKLLSSDEMWMYAPIAYNGMNIGLDL
NIDT*
Rice OsRSLh nucleotide sequence (SEQ ID NO: 71; LOC_Os12g39850.1 11982.m07769)
ATGGAGGGTGGAGGACTGATCGCCGATATGAGCTGGACCGTCTTCGACTTGCCATCGCACAGCGATGAGTCGGAG
ATGATGGCGCAGCTCTTCAGTGCATTCCCCATCCATGGTGAGGAGGAAGGCCATGAGCAGCTCCCATGGTTTGAT
CAATCTTCCAATCCATGCTACTATAGCTGCAATGCTAGCAGCACTGCATACAGCAACAGCAATGCTAGTAGCATT
CCTGCTCCATCTGAGTATGAAGGATACTGCTTCAGTGACTCAAATGAGGCCCTGGGTGTCAGCTCCAGCATTGCA
CCACATGACCTGAGCATGGTCCAGGTGCAAGGTGCAACTGAGTTTCTGAATGTGATCCCAAACCATTCCCTTGAT
TCATTCGGTAATGGCGAGCTGGGCCACGAGGATCTTGATTCGGTTAGTGGGACTAACAAGAGAAAACAGTCGGCA
GAAGGAGAATTTGATGGCCAAACAAGAGGTTCAAAATGCGCGAGAAAGGCTGAACCGAAGCGAGCGAAGAAGGCC
AAGCAAACTGTGGAGAAGGATGCAAGTGTTGCCATCCCAAATGGGAGCTGTTCCATTTCTGACAATGATTCCAGT
TCATCCCAGGAGGTTGCAGATGCTGGTGCTACTTCGAAAGGCAAATCCCGGGCTGGCCGCGGAGCAGCCACTGAT
CCCCAGAGCCTCTATGCAAGGAAAAGGAGAGAGAGGATCAATGAGAGGCTCAAGACACTTCAGAACCTTGTGCCC
AATGGCACCAAAGTTGATATCAGCACCATGCTTGAGGAGGCAGTCCACTATGTGAAGTTCCTGCAGCTTCAGATC
AAGCTCCTCAGCTCCGATGAAATGTGGATGTATGCGCCAATTGCGTACAACGGGATGAACATTGGGCTCGATCTG
AACATTGATACATGA
Rice OsRSLi amino acid sequence (SEQ ID NO: 72; LOC_Os07g39940.1 11977.m08236)
MAQFLGAHGDHCFTYEQMDESMEAMAAMFLPGLDTDSNSSSGCLNYDVPPQCWPQHGHSSSVTSFPDPAHSYGSF
EFPVMDPFPIADLDAHCAIPYLTEDLISPPHGNHPSARVEEATKVVTPVATKRKSSAAMTASKKSKKAGKKDPIG
SDEGGNTYIDTQSSSSCTSEEGNLEGNAKPSSKKMGTRANRGAATDPQSLYARKRRERINERLRILQNLVPNGTK
VDISTMLEEAVQYVKFLQLQIKLLSSDDTWMYAPIAYNGVNISNIDLNISSLQK*
Rice OsRSLi nucleotide sequence (SEQ ID NO: 73; LOC_Os07g39940.1 11977.m08236)
ATGGCGCAGTTTCTTGGAGCTCATGGTGATCACTGCTTCACCTACGAGCAAATGGATGAGTCCATGGAGGCAATG
GCAGCGATGTTCTTGCCTGGCCTTGACACCGACTCCAATTCTTCTTCTGGTTGTCTCAACTACGATGTGCCTCCA
CAATGCTGGCCTCAGCATGGCCATAGCTCTAGCGTCACCAGCTTCCCTGATCCAGCTCATAGCTATGGAAGCTTT
GAGTTCCCGGTCATGGATCCGTTCCCGATCGCCGATCTCGACGCGCATTGCGCCATCCCCTACCTTACTGAGGAT
CTGATCAGCCCTCCACATGGCAACCATCCATCAGCAAGAGTGGAAGAAGCTACAAAGGTTGTTACACCAGTGGCT
ACCAAGAGGAAGTCTAGTGCTGCCATGACGGCATCAAAGAAGAGCAAGAAGGCTGGCAAAAAAGATCCTATTGGC
AGCGACGAAGGCGGCAACACCTACATTGATACGCAAAGTTCTAGCAGTTGCACCTCAGAGGAAGGAAACCTGGAG
GGCAACGCGAAGCCGAGCTCGAAGAAGATGGGTACTAGGGCCAACCGTGGGGCGGCAACCGATCCCCAGAGTCTC
TATGCAAGGAAGAGGAGAGAGAGGATCAATGAAAGATTGAGGATCCTGCAGAACTTGGTTCCCAATGGAACAAAG
GTTGACATCAGTACAATGCTGGAGGAAGCAGTGCAGTATGTCAAATTTTTGCAACTTCAGATTAAGTTGCTAAGC
TCTGATGACACGTGGATGTATGCACCAATCGCTTACAATGGAGTCAACATCAGCAATATTGATCTGAACATCTCT
TCTCTGCAAAAATAA
Populus trichocarpa PtRSLa amino acid sequence (SEQ ID NO: 74)
MALAKDRMGSVQTCPYNGNVMGDFSSMGSYGFDEYQKVAFYEEGNSTFEKTSGLMIKNLAMTSSPSSLGSPSSAI
SGELVFQATDHQAEEAHSLISFKGIGFDNIMHNNGSLLSFEQSSRVSQTSSQKDDYSAWEGNLSYNYQWNEMNPK
CNTSPRLMEDFNCFQRAGNFISMTGKENHGDWLYAESTIVADSIQDSATPDASSFHKRPNMGESMQALKKQCNNA
TKKPKPKSAAGPAKDLQSIAAKNRRERISERLKVLQDLVPNGSKVDLVTMLEKAISYVKFLQLQVKVLATDELWP
VQGGKAPDISQVKEAIDALLSSQTKDGNSSSSPK*
Populus trichocarpa PtRSLa nucleotide sequence (SEQ ID NO: 75)
ATGGCACTTGCCAAGGACCGTATGGGATCGGTTCAAACTTGCCCCTATAATGGAAATGTGATGGGGGATTTTTCC
TCCATGGGGTCTTACGGATTTGATGAATATCAGAAGGTAGCATTTTATGAAGAGGGAAATAGCACCTTTGAGAAA
ACCAGTGGGCTTATGATCAAGAATTTAGCTATGACCTCTTCTCCTTCTTCTCTTGGCAGTCCGAGCAGCGCGATT
TCTGGTGAATTAGTGTTTCAGGCTACTGACCATCAAGCTGAGGAAGCTCATTCTTTGATCAGCTTCAAAGGTATC
GGATTCGATAACATCATGCATAATAATGGATCTTTGCTTAGCTTTGAGCAAAGTAGTAGGGTTTCTCAAACTAGT
AGCCAGAAAGATGACTACTCAGCCTGGGAGGGTAATTTGAGTTACAACTACCAGTGGAACGAAATGAATCCAAAA
TGTAACACAAGTCCTCGGTTGATGGAAGATTTTAATTGCTTTCAAAGAGCTGGCAACTTCATTTCCATGACTGGA
AAGGAAAATCATGGTGATTGGTTATACGCTGAATCCACAATTGTTGCTGATAGCATTCAGGATTCTGCAACACCA
GATGCCAGCAGCTTCCATAAGCGTCCTAATATGGGAGAGAGTATGCAGGCTCTAAAGAAGCAATGCAACAATGCA
ACAAAAAAGCCAAAACCGAAGTCCGCAGCAGGTCCAGCTAAGGATCTACAGAGTATTGCTGCCAAGAATCGACGA
GAGAGGATTAGCGAGAGGCTTAAGGTATTGCAGGATTTAGTCCCTAATGGCTCAAAGGTTGATTTGGTTACTATG
CTAGAGAAAGCCATTAGTTATGTTAAGTTTCTTCAATTGCAAGTAAAGGTGTTAGCCACTGATGAATTATGGCCA
GTTCAAGGTGGTAAAGCTCCTGATATTTCTCAAGTAAAGGAAGCCATCGATGCCCTACTCTCATCTCAGACTAAA
GACGGAAACTCAAGCTCAAGCCCAAAGTAA
Populus trichocarpa PtRSLb amino acid sequence (SEQ ID NO: 76)
MALAKDRMDSVQTCALYGNVMGDLSSLGPNYRFDEEGDRNFEKNSALMIKNLAMSPSPPSLGSPSSANSGELVFQ
ATDNQVEEAHSLINFKGTGFDSIMHANGSLISFEQSNRVSQTSSHKDDYSAWEGNLSCNYQWNQINPKCNANPRL
MEDLNCYQSASNFNSITNSAEKENHGDWLYTHESTIVTDSIPDSATPDASSFHKRPNMGESMQALKKQRDSATKK
PKPKSAGPAKDPQSIAAKNRRERISERLKMLQDLVPNGSKVDLVTMLEKAISYVKFLQLQVKVLATDEFWPVQGG
KAPDISQVKGAIDATLSSQTKDRNSNSSSK*
Populus trichocarpa PtRSLb nucleotide sequence (SEQ ID NO: 77)
ATGGCACTTGCCAAGGACCGTATGGATTCGGTTCAAACTTGCGCCCTTTATGGAAATGTGATGGGGGATCTTTCC
TCCTTGGGGCCTAATTATAGATTTGATGAAGAGGGAGATAGGAACTTTGAGAAAAATAGTGCGCTTATGATCAAG
AATTTAGCTATGAGCCCTTCTCCTCCTTCTCTTGGCAGTCCAAGCAGTGCAAATTCTGGTGAACTAGTGTTTCAG
GCTACTGACAATCAAGTTGAGGAAGCTCATTCTTTGATCAACTTCAAAGGTACCGGATTTGATAGTATCATGCAT
GCTAATGGATCTTTGATTAGCTTTGAGCAAAGTAATAGGGTTTCTCAAACTAGTAGTCACAAAGATGACTACTCT
GCTTGGGAGGGTAATTTGAGTTGCAATTACCAGTGGAACCAAATCAATCCAAAATGTAACGCAAATCCTCGGTTG
ATGGAAGATCTTAATTGCTATCAAAGTGCAAGCAACTTCAACTCCATAACCAACAGTGCTGAAAAGGAAAACCAT
GGTGATTGGTTATACACTCATGAATCCACAATTGTTACTGATAGCATTCCCGATTCTGCAACACCAGATGCCAGC
AGCTTCCATAAGCGTCCCAATATGGGAGAGAGTATGCAGGCTCTAAAGAAGCAACGCGACAGCGCCACAAAAAAG
CCGAAACCCAAGTCTGCTGGTCCAGCTAAGGATCCACAAAGTATTGCTGCCAAGAATCGACGAGAGCGGATTAGC
GAGCGCCTTAAGATGTTGCAGGATTTAGTCCCTAACGGCTCCAAGGTTGATTTGGTTACTATGCTAGAGAAAGCC
ATTAGTTATGTTAAGTTTCTTCAATTGCAAGTAAAGGTGTTGGCCACTGATGAATTCTGGCCAGTTCAAGGTGGT
AAAGCTCCTGATATTTCTCAAGTAAAGGGAGCCATTGATGCCACACTCTCATCTCAGACTAAAGACAGAAATTCA
AACTCAAGCTCAAAGTGA
Populus trichocarpa PtRSLc amino acid sequence (SEQ ID NO: 78)
MAEGEWSSLGGMYTSEEADFMAQLLGNCPNQVDSSSNFGVPSSFWPNHEPTTDMEGANECLFYSLDFANINLHHF
SQGSSSYSGGSGILFPNTSQDSYYMSDSHPILANNNSSMSMDFCMGDSYLVEGDDCSNQEMSNSNEEPGGNQTVA
ALPENDFRAKREPEMPASELPLEDKSSNPPQISKKRSRNSGDAQKNKRNASSKKSQKVASTSNNDEGSNAGLNGP
ASSGCCSEDESNASHELNRGASSSLSSKGTATLNSSGKTRASRGAATDPQSLYARKRRERINERLRILQTLVPNG
TKVDISTMLEEAVQYVKFLQLQIKLLSSEDLWMYAPIAYNGMDIGLDHLKVTAP*
Populus trichocarpa PtRSLc nucleotide sequence (SEQ ID NO: 79)
ATGGCAGAGGGAGAGTGGAGTTCTCTTGGTGGAATGTACACTAGTGAGGAGGCTGATTTCATGGCACAGTTGCTT
GGTAACTGTCCTAATCAGGTTGATTCAAGTTCAAACTTTGGAGTTCCATCTAGTTTCTGGCCTAACCACGAACCA
ACAACGGACATGGAAGGGGCTAATGAATGTTTATTTTATTCTTTGGATTTTGCTAATATTAATTTGCACCATTTT
TCACAAGGGAGTAGTAGTTATAGTGGTGGCAGTGGCATTCTTTTTCCCAACACAAGCCAAGATAGCTACTACATG
AGTGATTCTCATCCAATTTTGGCTAACAATAATAGCTCAATGTCAATGGATTTTTGCATGGGAGACTCATATCTC
GTTGAAGGCGATGACTGCTCAAACCAAGAAATGAGCAATAGCAATGAGGAGCCTGGTGGAAACCAGACTGTAGCT
GCTCTTCCTGAAAACGATTTTCGGGCCAAGAGAGAACCAGAGATGCCAGCTTCTGAACTACCCCTGGAAGACAAA
AGCAGCAACCCACCTCAGATTTCTAAGAAAAGATCACGAAATTCAGGAGATGCTCAAAAGAACAAGAGGAATGCA
AGTTCAAAGAAGAGCCAGAAGGTTGCCTCGACTAGCAACAATGATGAAGGAAGTAATGCTGGCCTTAATGGGCCT
GCCTCAAGCGGTTGCTGCTCAGAGGATGAATCCAATGCCTCTCATGAGCTCAATAGAGGAGCGAGTTCAAGTTTG
AGCTCGAAAGGGACTGCAACTCTCAACTCAAGTGGCAAAACAAGAGCCAGCAGGGGGGCAGCCACTGATCCCCAG
AGTCTCTATGCAAGGAAAAGAAGAGAAAGAATAAATGAGAGGCTGAGAATTCTACAAACCCTTGTCCCCAACGGA
ACAAAGGTTGACATTAGCACAATGCTTGAAGAAGCTGTCCAGTATGTGAAGTTTTTGCAACTCCAAATTAAGCTG
CTAAGCTCTGAGGACTTGTGGATGTATGCGCCTATCGCTTACAACGGGATGGACATCGGTCTTGATCATCTGAAG
GTTACCGCACCATGA
Populus trichocarpa PtRSLd amino acid sequence (SEQ ID NO: 80)
MEPIGATAEGEWSSLSGMYTSEEADFMEQLLVNCPPNQVDSSSSFGVPSSFWPNHESTMNMEGANECLLYSLDIA
DTNLYHFSQVSSGYSGELSNGNVEESGGNQTVAALPEPESNLQPKRESKMPASELPLEDKSRKPPENSKKRSRRT
GDAQKNKRNVRSKKSQKVASTGNNDEESNGGLNGPVSSGCCSEDESNASQELNGGASSSLSSKGTTTLNSSGKTR
ASKGAATDPQSLYARKRRERINERLRILQNLVPNGTKVDISTMLEEAVQYVKFLQLQIKLLSSEDLWMYAPIAYN
GMDIGLDHLKLTTPRRL*
Populus trichocarpa PtRSLd nucleotide sequence (SEQ ID NO: 81)
ATGGAGCCTATTGGAGCCACTGCGGAGGGAGAGTGGAGTTCTCTTAGTGGAATGTACACAAGTGAGGAGGCTGAT
TTCATGGAACAGTTGCTTGTCAACTGTCCTCCTAATCAGGTTGATTCAAGTTCAAGCTTTGGAGTTCCATCTAGT
TTTTGGCCTAACCATGAATCAACAATGAACATGGAAGGGGCCAATGAATGTTTATTGTATTCTTTGGATATTGCT
GATACTAATCTGTACCATTTTTCACAAGTGAGCAGTGGTTATAGTGGTGAATTGAGCAATGGAAATGTGGAAGAG
TCTGGTGGAAACCAGACTGTAGCTGCTCTTCCTGAACCTGAAAGCAATTTGCAACCCAAGAGAGAATCAAAGATG
CCAGCATCTGAACTACCCCTGGAAGATAAAAGCAGAAAGCCACCTGAGAATTCCAAGAAAAGATCACGACGTACG
GGAGATGCCCAAAAGAACAAGAGGAATGTAAGGTCAAAGAAGAGCCAGAAGGTTGCCTCGACTGGCAACAATGAT
GAAGAAAGCAATGGTGGCCTTAATGGTCCTGTCTCAAGCGGTTGCTGCTCAGAGGATGAATCCAATGCCTCCCAG
GAGCTCAATGGAGGAGCGAGTTCAAGTTTGAGCTCAAAAGGGACAACAACTCTCAACTCAAGTGGCAAAACAAGA
GCCAGTAAGGGGGCAGCCACTGATCCCCAGAGCCTCTATGCAAGGAAAAGAAGAGAAAGAATAAATGAGAGGCTG
AGAATTCTACAAAACCTTGTCCCCAATGGAACAAAGGTTGACATTAGCACAATGCTTGAAGAGGCTGTCCAGTAT
GTGAAGTTTTTGCAACTCCAAATTAAGCTGCTAAGCTCTGAAGACCTGTGGATGTATGCTCCTATCGCGTACAAT
GGTATGGACATCGGTCTTGATCATCTGAAGCTTACCACACCAAGACGATTGTAG
Populus trichocarpa PtRSLe amino acid sequence (SEQ ID NO: 82)
MNTQAMEAFRDGELWNFSRMFSMEEPDCTPELLGQCSFLQDTDEGLHFTIPSAFFPAPESDASMAEDESLFYSWH
TPNPNLHFDSQESSNNSNSSSSVFLPYSSHESYFFNDSNPIQATNNNSMSMDIMDEENIGLEMPLFPEIAMAETA
CMNGDMSGDKTGDLDDNLKPAANDVLAKGLQLKRKLDVPEPIANTLDDMKKKARVTRNVQKTRKVGQSKKNQKNA
PDISHDEEESNAGPDGQSSSSCSSEEDNASQDSDSKVSGVLNSNGKTRATRGAATDPQSLYARKRRERINERLKI
LQNLVPNGTKVDISTMLEEAVHYVNFLQLQIKLLSSDDLWMYAPLAYNGIDIGLNQKLSMFL*
Populus trichocarpa PtRSLe nucleotide sequence (SEQ ID NO: 83)
ATGAATACGCAGGCTATGGAAGCCTTTCGTGATGGAGAATTATGGAACTTCAGCAGAATGTTCTCCATGGAAGAG
CCTGATTGCACCCCAGAATTACTTGGTCAGTGCTCTTTTCTTCAGGATACTGATGAAGGATTGCATTTTACAATC
CCATCAGCTTTCTTCCCTGCTCCTGAATCCGACGCGAGCATGGCTGAGGACGAGAGTTTGTTTTATTCTTGGCAT
ACTCCCAACCCCAATTTGCATTTTGATTCTCAAGAAAGTAGTAATAACAGTAATTCTAGCAGTAGTGTATTTCTT
CCCTATTCCAGCCATGAATCCTACTTCTTCAATGATTCTAATCCCATTCAAGCTACGAACAATAACTCTATGTCC
ATGGATATTATGGATGAGGAAAATATTGGCTTGTTTATGCCACTTTTTCCTGAAATTGCAATGGCAGAAACTGCC
TGTATGAATGGAGATATGAGCGGTGACAAAACAGGAGATTTAGATGATAATCTGAAGCCAGCAGCTAATGATGTT
CTGGCCAAGGGATTGCAGCTCAAAAGGAAGCTTGATGTTCCAGAACCAATAGCCAACACATTGGACGACATGAAG
AAAAAAGCCCGGGTTACAAGAAATGTGCAAAAGACTAGGAAGGTTGGACAGTCAAAAAAAAATCAGAAGAACGCA
CCAGATATTAGCCATGATGAAGAAGAGAGTAATGCTGGACCAGACGGACAAAGTTCCAGCAGTTGTAGTTCAGAA
GAGGACAATGCCTCTCAGGATTCTGATTCCAAGGTTTCTGGAGTTCTCAATTCCAATGGAAAAACAAGAGCTACT
AGGGGAGCTGCCACAGACCCCCAGAGCCTTTATGCAAGGAAAAGAAGGGAGAGGATAAACGAGAGACTGAAAATC
TTGCAGAATCTTGTCCCTAACGGAACCAAGGTTGATATCAGCACGATGCTAGAAGAGGCAGTCCATTACGTAAAC
TTTTTGCAGCTTCAAATCAAGCTTTTGAGCTCGGATGATCTATGGATGTATGCACCTCTGGCTTACAATGGAATA
GATATTGGACTCAACCAGAAGCTCTCTATGTTTCTATGA
Musa acuminata MaRSLa amino acid sequence (SEQ ID NO: 84; GI102139852,
ABF70010.1)
MAQESTWSSFDATMLAEEESRMIAQLLSNYQCFGEQDRDVGCCELPPSSCCSSHAADSCYCWSANENSNPGLCYW
SQSGDESDGAHAIGTVPVFTNHCLVGDQVAVNQTLSIHEPTAAHAEMPKRKIESHASEDDFRRQSSKKKLQAPTN
ALKSVKKARPGRNQKSIVCGDEEENNARSSGRSCCSYSSEEDSQAFQADLNAKTRSNRWPATDPQSLYAKQRRER
INARLRTLQNLVPNGTKVDISTMLEEAVRYVKFLQLQIKLLSSDELWMYAPVVHSGMIDGQVNSEIFVSANTRNE
WF*
Musa acuminata MaRSLa nucleotide sequence (SEQ ID NO: 85).
atggctcaggagtcaacttggagctcgtttgatgctacaatgcttgctgaggaggagtcccgaatgatcgcacaa
ttgctcagcaactaccagtgttttggcgagcaagatcgagatgttggatgctgtgaactcccgccatcgtcttgt
tgttcttctcatgcagctgattcatgttactgttggtcagcaaatgagaacagtaacccgggtttgtgctactgg
tctcagagtggagatgaatccgatggagcacatgcaatcggcactgtgccggtcttcacgaaccattgcttggtg
ggagatcaagtcgctgtgaatcaaactttgagcattcacgaacctactgctgctcatgcagagatgccaaagcgc
aagatagagtctcatgcttctgaagatgatttccgtcgtcaaagttctaagaaaaagcttcaggctccgacgaat
gctctgaagagcgtgaagaaggcacgacctgggaggaaccagaagagcattgtgtgtggtgatgaggaagagaac
aatgccaggagcagtggccggagttgctgcagctacagctctgaggaagactcacaagctttccaggctgatctt
aatgcaaaaacacgatcgaatcgatggccagccacagatcctcaaagcctctatgcaaagcaaagaagggaaaga
atcaatgctagattgaggacattgcagaacctggtgcctaatggaactaaagttgacattagcacaatgctcgaa
gaagctgttcgttacgtcaagttcttgcagctgcagataaagcttttgagctcggatgagctgtggatgtacgct
cctgttgtccacagtgggatgattgatggccaagtcaactcagagatatttgtgtctgcaaatactcgtaatgag
tggttctga
Medicago truncatula MtRSLa amino acid sequence (SEQ ID NO: 86; AC140548.11
GI:156231148)
MEPIGTFPEGEWDFFRKMFASEDHEYYSQQFLDQNSLLLGENDGLNNGTQSTFCTAEIGENERMFYSFDHAHIQN
SNYIPQTQENSYNSNSSASDDTNYYFSYPNHVLENNINNCISNDFRMDENLFASSVPSLNEIVMEENVRMNEDSA
SDDHIVEKNGYNTQIMEPFDLHTKHEMQMKLKRKLDVIEVEVPVEEKINNNPKKKPRVSNDGQGCMKNARSKKNH
KVIASHEEEMTEEINRGSNGNSSSSNISEDDNASQENSGGTTLNSNGKTRASRGSATDPQSLYARKRRERINERL
RVLQNLVPNGTKVDISTMLEEAVNYVKFLQTQIKLLSSDDMWMYAPLAYNGLDLGLNLNLNSSLPL*
Medicago truncatula nucleotide sequence (SEQ ID NO: 87, AC140548.11
GI:156231148)
atggaacctataggtactttccctgaaggagaatgggatttctttcgcaaaatgtttgcaagtgaagatcatgaa
tattactcacaacaatttcttgatcaaaattcacttcttctaggggaaaatgatgggttgaacaatggaacacag
tccacattttgcactgctgaaattggtgaaaatgagcgtatgttttattcttttgatcatgctcatatccaaaac
tctaactatattcctcaaactcaagagaatagttacaatagcaattctagtgctagtgatgatacaaattactat
tttagttatcctaatcatgtactagaaaataatattaataattgtatatccaatgattttcgcatggatgagaat
ttgtttgcttcttctgttccatcccttaatgagattgtaatggaagagaatgtgagaatgaatgaagattctgca
agtgatgatcatattgtggagaaaaatggttacaatactcaaataatggaaccttttgatcttcacaccaagcat
gagatgcaaatgaagctcaaaaggaaacttgatgtgatagaagtggaggttcccgttgaagaaaaaattaacaac
aatccgaagaaaaaacctcgtgtttcgaatgatggccaaggatgcatgaaaaatgcaaggtcaaagaagaaccac
aaagttattgctagccatgaagaggagatgacagaagagattaatagaggatcaaatggaaatagttctagtagt
aacatttctgaggatgataatgcttctcaagaaaatagtggaggaactactctcaactcaaatgggaagacaaga
gctagtagaggatctgcaacagatccccaaagtctatatgcaaggaaaagaagagagagaataaatgaacgacta
agagtcttacaaaatcttgtaccaaacggaacaaaggttgatatcagtacaatgcttgaagaggcagtcaattat
gtgaaatttttacagactcaaatcaagcttttgagctctgatgatatgtggatgtatgcaccacttgcttacaat
ggacttgaccttggactcaatctcaacctdaacagctctctaccactatga
Soybean GmRSLa amino acid sequence (SEQ ID NO: 88)
(gi|26056905|gb|CA799819.1|CA799819)
XFLCFSQGSSSSTDNSGNNIFSITSSGAYSCDPEANFDSVSMVLCLGDAKFSPHSFQCDDNSNQQINENTDEESS
LDPWKLAIADNNLQAKREYEMMVSEPVEVDRSRNLENLAKRLKSSIEVSKTLRSAKSGKNSKSASVSNDEDDRSL
SLQAQRNSCFSQSDSNAYLEPNGGASKDPAPPNLHRKSRATTGAATDPQSLYARKRRERINERLRILQNLVPNGT
KVDISTMLEEAVQYVKFLQLQIKLLSSDDLWMY
Soybean GmRSLa nucleotide sequence (SEQ ID NO: 89)
(gi|26056905|gb|CA799819.1|CA799819)
ATTTTTTGTGTTTCTCACAAGGGAGTAGCTCCAGTACTGATAATAGTGGTAATAATATCTTTTCCATTACAAGTA
GTGGAGCCTACTCCTGTGATCCAGAAGCAAACTTTGATTCTGTGTCCATGGTTTTGTGCCTTGGAGATGCCAAAT
TTAGTCCCCATAGTTTTCAATGTGATGACAACTCAAACCAACAGATAAATGAAAACACTGATGAAGAGTCAAGTC
TAGACCCATGGAAGTTGGCTATAGCTGACAATAATTTGCAGGCTAAGAGGGAGTATGAAATGATGGTTTCTGAAC
CTGTAGAAGTGGATAGAAGCAGAAACCTGGAGAACCTAGCAAAAAGACTAAAGAGTTCAATAGAGGTTTCAAAAA
CATTGAGGAGTGCTAAATCAGGGAAAAATTCAAAATCTGCTTCAGTGAGCAACGATGAAGATGATAGAAGCTTGA
GCCTCCAAGCCCAAAGGAATAGCTGTTTTTCACAGAGTGACTCTAATGCTTATCTGGAGCCAAATGGAGGGGCAT
CAAAAGATCCTGCACCTCCCAATTTGCATAGAAAATCAAGAGCAACTACCGGTGCTGCCACTGATCCACAGAGCC
TCTATGCAAGAAAGAGAAGAGAAAGAATAAATGAAAGGTTGAGAATACTGCAAAATCTTGTTCCCAACGGAACTA
AGGTGGATATCAGCACCATGCTTGAGGAAGCTGTCCAATACGTGAAGTTTTTACAGCTCCAAATTAAGCTTCTGA
GCTCTGACGATCTGTGGATGTAT
Soybean GmRSLb amino acid sequence (SEQ ID NO: 90)
(gi|15663066|gb|BI700437.1|BI700437)
XNLENLPKRLKSSIEVPKTSRNAKSRKNSKSASTSNDEDDRSLSLQVQRNNSCFSQSDSNAYLEPNGGASKDPAP
PNLDRKSRATTSAAADPQSLYARKRRERINERLRILQNLVPNGTKVDISTMLEEAVQYVKFLQLQIKLLSSEDLW
MYAPIVYNGINIGLDLGISPTKGRSM*
Soybean GmRSLb nucleotide sequence (SEQ ID NO: 91).
(gi|15663066|gb|BI700437.1|BI700437)
GAAACCTGGAGAACCTACCAAAAAGACTAAAGAGCTCAATAGAGGTCCCAAAAACATCGAGGAATGCTAAATCAA
GGAAAAATTCAAAATCTGCTTCAACTAGCAACGATGAAGATGATAGAAGCTTGAGCCTCCAAGTCCAAAGGAATA
ATAGCTGTTTTTCACAGAGTGACTCTAATGCTTATCTTGAGCCAAATGGAGGGGCATCAAAAGATCCTGCACCTC
CTAATTTGGATAGAAAATCAAGAGCAACTACCAGTGCCGCCGCTGATCCACAGAGCCTCTATGCAAGAAAGAGAA
GAGAAAGAATAAATGAAAGGCTGAGAATACTGCAAAATCTTGTCCCCAACGGAACTAAGGTGGATATCAGCACCA
TGCTTGAAGAAGCTGTCCAATACGTTAAGTTTTTACAGCTCCAAATTAAGCTTCTGAGCTCTGAAGATTTGTGGA
TGTATGCTCCAATTGTTTACAATGGAATAAACATTGGACTAGACCTCGGTATTTCTCCAACCAAAGGAAGATCAA
TGTGATAGCATAGCAATTAAAGAGGATATAATATTTCATTAACTTA
Lettuce saligna LsRSLa amino acid sequence (SEQ ID NO: 92)
(gi|83790803|gb|DW051020.1|DW051020 CLLX3812.b1_H18.ab1)
XRSKEAEILSSNGKRKASRGSATDPQSVYARKRRERINERLRILQNLVPNGTKVDISTMLEEAVEYVKFLQLQIK
LLSSDDMWMYAPIAYDGMDIGLHSTTIPSSSTR*
Lettuce saligna LsRSLa nucleotide sequence (SEQ ID NO: 93)
(gi|83790803|gb|DW051020.1|DW051020 CLLX3812.b1_H18.ab1)
TGAGATCAAAAGAGGCTGAAATTCTGAGCTCAAATGGCAAGAGAAAAGCAAGTAGGGGGTCAGCAACTGATCCAC
AAAGTGTCTATGCACGGAAAAGAAGAGAAAGAATTAACGAACGTTTAAGAATATTACAAAATCTTGTTCCTAATG
GTACAAAGGTTGATATAAGCACAATGCTTGAAGAGGCTGTTGAGTACGTGAAGTTTTTGCAGCTTCAAATCAAGC
TCTTGAGCTCCGATGATATGTGGATGTATGCTCCGATTGCATACGATGGAATGGACATTGGGCTTCATTCAACAA
CCATCCCATCATCGTCAACAAGATAATGCAAAGTTGGGCTATCCATATTGTCACATTTTTGTTGAATAAAAGGCA
ATCGATAACAAAATTCAAAGTTTATAAAGAGTACACATTTATGC
Triticum aestivum TaRSLa amino acid sequence (SEQ ID NO: 94)
(gi|25232820|gb|CA654295.1|CA654295)
MASKRATTRELRAMYDDEPSSMSLELFGYHGVVVDGDDENDDTATALPQLSFVDNFKGGCGSAADYYSWAYNASG
GTPGASSSSTSSVLSFEHAGGAGHQLAYNSGTGDDDCALWMDSMADHQHGAARFGFMNPGSADVVPEIQESSIKQ
PAKSAQKRSSSGGEAQAAAKKQCGGGRKSKAKVVPTKDPQSAVAKVRRERISERLKVLQDLVPNGTKVDMVTMLE
KAITYVKFLQLQVKVLATDEFWPVQGGKAPELSQVKTALDAILSSQQQP*
Triticum aestivum TaRSLa nucleotide sequence (SEQ ID NO: 95)
(gi|25232820|gb|CA654295.1|CA654295)
ATGGCGAGCAAGCGGGCCACCACGCGGGAGQTCCGGGCGATGTACGACGACGAGCCCTCCTCCATGTCCCTCGAG
CTCTTCGGCTACCATGGCGTGGTCGTCGACGGTGACGATGAAAACGACGACACTGCCACCGCCCTGCCCCAGCTC
TCCTTCGTCGACAACTTCAAAGGTGGGTGCGGGTCGGCGGCGGACTACTACAGCTGGGCGTACAACGCCTCCGGC
GGGACGCCGGGCGCCTCCTCCAGCTCCACCTCGTCGGTGCTCAGCTTTGAGCATGCCGGCGGTGCCGGTCATCAG
CTGGCTTATAATTCCGGCACAGGCGACGATGACTGCGCGCTCTGGATGGACAGCATGGCCGATCATCAGCACGGC
GCGGCCAGGTTTGGGTTCATGAACCCAGGGTCGGCCGATGTCGTCCCAGAAATCCAGGAGAGCAGCATCAAGCAG
CCGGCCAAGTCTGCGCAGAAGCGCTCGAGCTCGGGTGGTGAGGCGCAAGCAGCGGCGAAGAAGCAGTGTGGAGGA
GGCAGGAAGAGCAAGGCCAAAGTTGTCCCTACCAAGGATCCTCAGAGCGCTGTTGCAAAGGTCCGAAGAGAGCGC
ATCAGTGAGAGGCTCAAAGTTCTGCAGGATCTTGTACCCAACGGCACGAAGGTGGACATGGTCACCATGCTCGAG
AAGGCAATCACCTATGTCAAGTTCCTGCAGCTGCAAGTCAAGGTGTTGGCGACCGACGAGTTCTGGCCGGTGCAA
GGAGGGAAGGCGCCGGAGCTCTCCCAAGTGAAGACCGCGCTGGACGCCATCCTTTCTTCCCAGCAGCAACCCTAG
Safflower Carthamus tinctorius CtRSLa amino acid sequence (SEQ ID NO: 96)
(gi|125399878|gb|EL411863.1|EL411863 CFFS9477.b1_I18.ab1)
DSQIIHPMPCDELHKSLI*LYHIRRRYPYWVFTDGESTSFARPLLNDSRIRGELLLTLSTTKHCKVTASSMRRSY
SMMHDHEKS*KIQRRKSQKLVSKGNESEADHDAVFGQIMKMCGSDNDSNWPRESSTSPRPKEAANLNSNGKTKAN
RGSATDPQSVYARKRRERINERLRILQSLVPNGTKVDISTMLEDAVQYVKFLQLQIKPLSSDDLWMYAPIAYNGM
ETGLDSTIPSPR*RLSKVAASFFLKKGKPGA
Safflower Carthamus tinctorius CtRSLa nucleotide sequence (SEQ ID NO: 97)
(gi|125399878|gb|EL411863.1|EL411863 CFFS9477.b1_I18.ab1)
GATTCACAGATAATCCACCCTATGCCGTGTGATGAACTCCACAAATCCTTAATTTAATTGTACCACATCAGGCGA
CGTTATCCATATTGGGTGTTCACTGATGGTGAAAGCACATCTTTCGCGCGACCTCTACTCAATGACTCAAGAATT
AGAGGTGAACTATTGCTTACACTATCTACTACTAAACATTGTAAAGTGACTGCCAGTTCTATGAGACGTTCGTAT
AGCATGATGCATGATCATGAGAAAAGCTAAAAGATACAGCGCAGAAAGAGCCAGAAGCTCGTTTCTAAAGGCAAC
GAAAGTGAAGCTGACCATGATGCAGTTTTTGGGCAAATAATGAAAATGTGTGGATCTGACAATGACTCGAATTGG
CCTCGGGAGTCGAGCACAAGTCCAAGACCAAAAGAGGCTGCAAATCTGAACTCAAATGGGAAGACAAAAGCAAAT
AGGGGGTCAGCAACGGATCCACAAAGTGTCTACGCACGGAAGAGAAGAGAACGAATTAATGAACGGTTAAGAATA
CTACAGAGTCTGGTTCCTAATGGTACAAAGGTTGATATAAGCACAATGCTTGAAGATGCTGTCCAGTATGTGAAA
TTTTTGCAGCTCCAAATCAAGCCGTTGAGCTCTGATGATCTGTGGATGTATGCCCCCATCGCGTACAACGGGATG
GAGACGGGGCTTGATTCTACGATCCCCTCGCCAAGGTGAAGACTATCCAAAGTTGCCGCATCTTTTTTCTTGAAA
AAAGGGAAGCCTGGGGCAA
BdRSLa amino acid sequence (SEQ ID NO: 98)
MALVREPMVLYDGGFDASEASAFDSIGCFGHGHGHDALLGGVDAAALFGGYAHDEPAGASASAYVKDGSHWAGVG
ASVLAFDRAARGHGAQAMATAAAQEEEECDAWIDAMDEDNGEAAPAPSIGFDPATGCFSLTQRPGAGARRPFGLL
FPSASGGAPSPDSAAPAPASRGSQKRPSAGIARAQDAEPRASKKQCGASRKTTAKAKSPAPAITSPKDPQSLAAK
NRREKISERLRTLQEMVPNGTKVDMVTMLEKAISYVKFLQLQVKVLATDEFWPAQGGMAPEISQVKEALDAILSS
QRGQFNCSS*
BdRSLa nucleotide sequence (SEQ ID NO: 99)
ATGGCATTAGTGCGGGAGCCGATGGTACTGTATGACGGCGGTTTCGACGCCTCGGAGGCGTCGGCATTCGACTCC
ATCGGCTGCTTCGGCCACGGCCACGGCCACGACGCGCTCCTAGGCGGCGTCGACGCGGCCGCGCTGTTCGGGGGC
TACGCGCACGACGAGCCGGCCGGCGCCAGCGCCAGCGCCTACGTGAAGGACGGCTCGCACTGGGCCGGCGTGGGT
GCGTCCGTGCTCGCGTTCGACCGTGCCGCTCGGGGCCACGGCGCGCAGGCCATGGCGACCGCGGCCGCTCAGGAG
GAGGAAGAATGCGACGCGTGGATCGACGCCATGGACGAGGACAATGGCGAGGCGGCGCCGGCGCCGTCCATCGGC
TTCGACCCGGCCACGGGCTGCTTCAGCCTCACGCAGCGGCCCGGCGCCGGCGCGCGGCGCCCGTTCGGGCTCCTG
TTCCCGAGCGCGTCCGGTGGCGCGCCCTCGCCCGACAGCGCCGCGCCAGCGCCGGCATCCCGCGGTTCCCAGAAG
CGGCCATCCGCCGGGATTGCGCGCGCGCAGGACGCGGAGCCGCGGGCCAGCAAGAAGCAGTGCGGCGCGAGCAGG
AAGACGACGGCCAAGGCGAAGTCGCCTGCGCCTGCCATCACCTCGCCCAAGGACCCGCAGAGCCTCGCTGCAAAG
AACCGGAGGGAGAAGATCAGCGAGCGGCTCCGGACGTTGCAGGAGATGGTGCCCAACGGCACCAAGGTGGACATG
GTCACCATGCTCGAGAAGGCCATCAGCTACGTCAAGTTCCTGCAGCTGCAAGTCAAGGTGCTCGCGACGGACGAG
TTCTGGCCGGCGCAGGGAGGGATGGCGCCGGAGATCTCCCAGGTGAAGGAGGCGCTCGACGCCATCCTGTCGTCG
CAGAGGGGGCAATTCAACTGCTCCAGCTAG
BdRSLb amino acid sequence (SEQ ID NO: 100)
MASRHATTREPHLRTMYDDEPSMSLELFGYHGVVVDGDDDGDTATDLPQLTFVDNFKGGCGSADYYGWAYSASGG
ASGACSSSSSSVLSFEQAGGAGHQLAYNAGTGDDDCALWMDGMADQHDTAKFGFMDPGMSDVSLEIQESSMKPPA
KMAQKRACQGGETQAAAKKQCGGSKKSKAKAAPAKDPQSAVAKVRRERISERLKVLQDLVPNGTKVDMVTMLEKA
ITYVKFLQLQVKVLATDDFWPVQGGKAPELSQVKDALDAILSSQNQS*
BdRSLb nucleotide sequence (SEQ ID NO: 101)
ATGGCAAGCAGGCACGCCACTACACGGGAGCCACACCTCCGGACCATGTACGACGACGAGCCATCCATGTCCCTC
GAGCTCTTCGGCTACCATGGCGTCGTCGTCGACGGTGACGACGATGGCGACACCGCCACCGACCTTCCCCAGCTC
ACCTTTGTTGACAACTTCAAAGGCGGGTGTGGGTCAGCCGACTACTACGGCTGGGCGTACAGCGCCTCCGGTGGT
GCGTCAGGCGCCTGCTCCAGCTCCAGCTCGTCGGTGCTCAGCTTTGAGCAGGCGGGTGGTGCCGGTCATCAGCTG
GCTTATAACGCCGGCACAGGTGACGATGACTGCGCGCTCTGGATGGACGGCATGGCTGACCAGCATGACACAGCC
AAGTTTGGGTTCATGGACCCAGGCATGTCTGATGTCAGCCTAGAAATCCAGGAGAGCAGCATGAAACCGCCGGCC
AAGATGGCACAGAAGCGCGCTTGCCAGGGTGGTGAGACGCAAGCAGCGGCGAAGAAGCAGTGTGGAGGAAGCAAG
AAGAGCAAGGCAAAAGCTGCCCCTGCCAAGGATCCTCAAAGCGCCGTTGCAAAGGTCCGAAGAGAGCGCATCAGC
GAGAGGCTCAAAGTTCTGCAGGATCTCGTGCCCAATGGCACAAAGGTTGACATGGTCACCATGCTCGAAAAGGCA
ATCACCTATGTCAAGTTCCTGCAGCTGCAAGTCAAGGTATTGGCGACTGATGACTTCTGGCCGGTGCAAGGAGGG
AAAGCTCCGGAGCTCTCCCAAGTGAAGGACGCTCTGGACGCGATCCTGTCTTCCCAGAATCAATCCTAG
BdRSLc amino acid sequence (SEQ ID NO: 102)
MALVGQATKLCYDGFAGDGVPPFMDAACLAFDHGYDYNNPHAWEFPTGAEPGNSSAFDVAWTGVSSTSPVLTFDA
AEWMDATATDRLSSYSPSAATVPASYKRPRAHVQPQQEAEEQESITPNPKKQCGDGKVVIKSSAAATGTSPRKEP
QSQAAKSRRERIGERLRALQELVPNGSKVDMVTMLDKAITYVKFMQLQLTVLETDAFWPAQGGAAPEISQVKAAL
DAIILSSSQKPRQWS*
BdRSLc nucleotide sequence (SEQ ID NO: 103)
ATGGCTCTAGTGGGTCAGGCAACGAAGCTCTGCTACGACGGCTTCGCCGGAGACGGTGTGCCGCCGTTCATGGAC
GCAGCTTGTCTGGCATTCGACCACGGGTATGATTACAACAATCCCCACGCATGGGAATTCCCCACCGGCGCCGAG
CCAGGCAACAGCAGCGCGTTCGACGTTGCCTGGACCGGCGTCTCCTCCACTTCTCCGGTGCTCACATTCGACGCC
GCCGAGTGGATGGACGCCACGGCCACGGACCGGCTGAGCTCCTACAGCCCGTCTGCGGCCACCGTGCCGGCCTCT
TACAAGCGGCCTCGTGCGCACGTGCAGCCACAGCAGGAAGCAGAAGAACAGGAAAGCATTACTCCCAATCCCAAG
AAGCAGTGCGGCGATGGGAAAGTAGTTATCAAGTCATCGGCGGCGGCTACCGGCACCAGTCCACGCAAGGAACCC
CAAAGCCAAGCTGCCAAGAGCCGTCGTGAGCGGATCGGCGAGCGGCTGAGAGCGCTGCAGGAGCTGGTGCCCAAC
GGCAGCAAGGTGGACATGGTCACCATGCTCGACAAGGCCATCACTTATGTCAAGTTCATGCAGCTCCAGCTCACG
GTGCTCGAGACAGACGCGTTCTGGCCTGCGCAGGGTGGCGCGGCGCCGGAGATCTCCCAGGTGAAGGCGGCGCTC
GACGCCATCATCCTCTCCTCGTCGCAGAAGCCTCGTCAGTGGAGCTAG
BdRSLd amino acid sequence (SEQ ID NO: 104)
MEAGGLISEAGWTMFDFPSQGEESEIMSQLLGAFPSHLEEGHQDLPWYQASDPSYYDCNLNTSSESNASSLAVPS
ECMGYYLGDSSESLDLSSCIAPNDLNLVQEQDATEFLNMTPNLSLDLRGNGESSCEDLTSVGPTNKRKHSSAEEG
IDCQARGQKFARKAEPKRTKKTKQSGWEVAVATRNGSTASCCTSDDDSNASQESADTGVCPKGKARAARGASTDP
QSLYARKRRERINERLKTLQTLVPNGTKVDMSTMLEEAVHYVKFLQLQIKVLSSDDMWMYAPLAYNGMNIGLDLN
IYTPERWRTASAAPSTEGREYAGVDRISDLPDGILGDIVSLLPTAEGARTQILKRRWRHIWRCSAPLNLDCCTLV
ARGGGREAEDELVGLIPSILSSHQGTGRRFHVPSSRHSDRAATIEAWLQSAALDNLQELDLWCTHTYLYDYVPLP
PAVFRFSATVRVVTIANCNLRDSAVQGLQFPQLKQLGFKDIIIMEDSLHHMIAACPDLECLMIERSLGFACVRIN
SLSLRSIGVSTDHPHPHELQFVELVIDNAPCLKRLLHLEMCYHLDMHITVISAPKLETLSCCSSVSRSSTKLSFG
SAAIQGLHIDSLTTVVRTVQILAVEMHSLCLDTIIDFMKCFPCLQKLYIKSFVSGNNWWQRKHRNVIKSLDIRLK
TIALESYGGNQSDINFVTFFVLNARVLELMTFDVCSEHYTVEFLAEQYRKLQLDKRASRAARFHFTSNRCVRGIP
YIGRAELFLPIKCSHVDTSPNLSSFRLSAVFSVCITRNLLRLKKAMWVISLYYSPEFTKQVAVHNPNEMPF*
BdRSLd nucleotide sequence (SEQ ID NO: 105)
ATGGAGGCTGGAGGGCTGATTTCTGAGGCTGGCTGGACCATGTTTGACTTCCCGTCGCAAGGCGAGGAATCAGAG
ATCATGTCGCAGCTGCTAGGCGCCTTCCCCTCCCATCTTGAGGAAGGCCATCAGGATCTGCCTTGGTACCAGGCT
TCTGACCCATCCTACTATGACTGTAATCTTAATACAAGTAGTGAAAGCAATGCTAGTAGTCTTGCTGTTCCATCC
GAGTGTATGGGCTACTATTTGGGTGATTCAAGTGAGTCCCTGGACCTGAGCTCCTGCATTGCACCAAATGACCTG
AACTTGGTCCAGGAGCAAGATGCAACTGAGTTTCTGAATATGACACCAAATCTTTCCCTTGATTTACGTGGGAAT
GGTGAGTCGAGCTGCGAGGATCTCACTTCGGTCGGTCCTACTAACAAGCGAAAGCACTCCTCGGCAGAAGAAGGA
ATCGACTGCCAAGCAAGAGGCCAGAAATTCGCCAGAAAGGCTGAACCGAAGCGAACAAAGAAGACCAAGCAAAGC
GGATGGGAGGTTGCTGTTGCCACCAGGAATGGAAGCACAGCGAGCTGCTGCACCTCTGATGATGACTCAAACGCT
TCTCAAGAATCTGCAGATACCGGTGTTTGTCCGAAAGGCAAGGCTCGGGCTGCCCGTGGCGCATCAACTGATCCC
CAGAGCCTCTATGCAAGGAAAAGGAGGGAAAGGATCAATGAGAGACTGAAGACACTGCAGACCCTTGTGCCCAAT
GGAACCAAAGTAGATATGAGCACCATGCTTGAGGAGGCAGTCCACTACGTGAAGTTCCTGCAGCTTCAGATCAAG
GTCTTGAGCTCTGATGATATGTGGATGTATGCGCCGCTAGCATACAACGGGATGAACATTGGGCTTGATCTGAAC
ATATATACTCCGGAGAGGTGGAGGACAGCGTCCGCGGCGCCCTCAACCGAAGGGCGTGAATACGCCGGCGTCGAC
CGCATCAGCGACCTCCCCGACGGCATCCTCGGCGACATCGTCTCGTTGCTCCCCACCGCCGAAGGAGCCCGCACC
CAGATCCTCAAGCGCAGGTGGCGCCACATCTGGCGCTGCTCCGCCCCTCTCAACCTCGATTGCTGTACCTTGGTC
GCCCGTGGCGGCGGCCGTGAGGCTGAAGATGAACTCGTCGGTCTCATACCGTCCATCCTTTCTTCTCACCAAGGC
ACCGGCCGCCGCTTCCACGTCCCCTCGTCGCGCCACTCTGACCGAGCTGCTACCATTGAAGCCTGGCTCCAATCT
GCTGCCCTCGACAATCTCCAGGAGCTCGATTTATGGTGCACCCACACCTATCTTTACGACTATGTTCCGCTGCCA
CCCGCCGTCTTTCGCTTCTCCGCCACCGTCCGTGTTGTCACCATCGCAAATTGTAACCTCCGTGACAGCGCCGTC
CAAGGCCTTCAATTCCCACAACTTAAACAGCTCGGATTCAAAGATATCATCATCATGGAGGATTCGCTGCACCAC
ATGATTGCTGCGTGTCCAGATCTCGAGTGCTTGATGATTGAAAGGAGCTTAGGTTTTGCTTGCGTCCGGATCAAT
TCCCTTAGTCTTAGAAGCATCGGTGTGAGCACTGACCACCCTCACCCACATGAGCTCCAGTTTGTGGAACTCGTC
ATTGATAATGCACCTTGTCTTAAGAGATTGCTCCATCTTGAAATGTGTTATCACCTTGACATGCATATAACAGTA
ATCTCCGCGCCTAAACTGGAGACCTTGAGCTGCTGTTCTTCTGTGAGTCGCTCCTCCACCAAACTCTCGTTTGGC
TCCGCGGCCATTCAGGGATTGCACATTGATAGCCTAACAACAGTGGTGCGCACTGTCCAAATTTTAGCTGTAGAG
ATGCATTCTCTTTGTCTAGACACAATTATTGACTTCATGAAATGCTTTCCATGTCTGCAGAAGTTGTACATTAAG
TCATTTGTAAGTGGAAACAATTGGTGGCAACGTAAACACCGGAACGTTATCAAATCCCTTGACATCCGTCTCAAG
ACAATAGCGTTGGAAAGTTATGGGGGCAATCAGTCTGACATCAACTTTGTCACATTCTTTGTCTTGAACGCGAGA
GTGCTAGAGTTGATGACATTTGACGTTTGTTCTGAGCATTACACTGTGGAGTTCTTGGCAGAGCAATATAGGAAG
CTTCAGCTAGATAAGAGGGCTTCAAGAGCCGCTCGGTTCCATTTTACAAGTAACCGATGTGTCCGTGGTATTCCG
TATATCGGACGTGCCGAGCTATTCTTGCCTATCAAATGTTCTCATGTTGACACCAGTCCAAACTTGAGTAGTTTC
CGTTTGTCTGCAGTATTTTCAGTTTGTATTACCCGGAACCTTTTGCGTTTAAAAAAAGCTATGTGGGTCATTAGT
TTGTATTATTCTCCAGAATTTACAAAACAAGTGGCCGTGCACAATCCCAATGAAATGCCGTTTTAG
BdRSLe amino acid sequence (SEQ ID NO: 106)
MEAKCGAIWSSIDARSEDSEMIAHLQSMFWSNSDVALNLCSSNTSGNSCVTASTLPSSLFLPLVDNESYGAAPSV
DTGMDSCFDHQHQSITGHKRISHMDEQMKKTRKKSRTVPSVSKALGSSLVDNQMNADIFNQSSSCCSSGEDSIGT
SEKSIVANQSDNTSGCKRPSKNMQSLYAKKRRERINEKLRVLQQLIPNGTKVDISTMLEEAVQYVKFLQLQIKVL
SSDETWMYAPLAYNGMDIGLTLALRTAANQE*
BdRSLe nucleotide sequence (SEQ ID NO: 107)
ATGGAGGCCAAGTGTGGAGCTATTTGGAGCTCTATCGATGCGAGGAGCGAGGACTCTGAGATGATTGCTCACCTG
CAGTCCATGTTCTGGAGCAACAGTGATGTTGCTCTCAACCTCTGTTCGTCAAACACCAGTGGCAATTCTTGTGTC
ACAGCTAGCACATTGCCTAGCAGCTTGTTCCTTCCTCTTGTCGATAATGAGAGCTATGGTGCAGCGCCATCGGTG
GACACCGGCATGGATTCATGCTTTGATCACCAGCATCAGAGCATTACTGGTCACAAGAGGATATCGCACATGGAT
GAGCAGATGAAGAAGACGAGAAAGAAGTCCCGGACTGTTCCATCGGTATCAAAGGCTCTGGGTTCCAGCCTAGTC
GATAATCAGATGAATGCTGACATTTTCAATCAGAGCTCCTCCTGCTGCAGCTCGGGAGAAGATTCAATTGGAACA
TCTGAGAAATCCATTGTTGCAAACCAGAGTGACAATACGAGTGGTTGTAAGCGGCCTTCAAAGAATATGCAAAGC
CTTTATGCAAAGAAGAGAAGAGAGAGGATCAACGAGAAGTTGAGAGTACTGCAGCAGCTGATTCCCAATGGCACC
AAAGTTGACATCAGCACAATGTTGGAGGAAGCAGTTCAGTATGTCAAGTTTCTGCAGCTGCAAATAAAGGTCTTA
AGCTCTGACGAGACATGGATGTATGCGCCCCTCGCCTACAATGGTATGGACATCGGTCTCACTCTCGCTCTGAGA
ACTGCTGCAAACCAAGAGTGA
Zea mays ZmRSLa amino acid sequence (AZM4_60871: SEQ ID NO: 108)
MALVREHGGYYGGFDSVEAAAFDTLGYGHGASLGFDASSALFGEGGYAAGGGDAWAGAGASTVLAFNRTTAAAAV
GVEEEEEECDAWIDAMDEDDQSSGPAAAAPEARHALTASVGFDASTGCFTLTERASSSSGGAGRPFGLLEPSTSS
SGGTPERTAPVRVPQKRTYQAVSPNKKHCGAGRKASKAKLASTAPTKDPQSLAAKQNRRERISERLRALQELVPN
GTKVDLVTMLEKAISYVKFLQLQVKVLATDEFWPAQGGKAPEISQVREALDAILSSAS
Zea mays ZmRSLa nucleotide sequence (AZM4_60871: SEQ ID NO: 109)
ATGGCGTTGGTGAGGGAGCACGGTGGGTACTACGGAGGCTTCGACAGCGTCGAGGCGGCGGCCTTCGACACGCTC
GGCTACGGCCACGGCGCGTCGCTGGGCTTTGACGCGTCGTCGGCGCTGTTCGGGGAAGGCGGTTATGCGGCGGGC
GGCGGGGACGCCTGGGCGGGCGCGGGGGCGTCGACCGTCCTGGCGTTCAACCGCACAACGGCAGCGGCGGCCGTG
GGTGTGGAAGAGGAGGAGGAGGAGTGCGACGCGTGGATCGACGCTATGGACGAGGACGACCAGAGCTCCGGCCCC
GCCGCGGCGGCGCCAGAGGCGCGCCACGCGCTGACGGCCTCCGTGGGTTTCGACGCCTCCACGGGGTGCTTCACC
CTGACGGAGAGGGCGTCGTCGTCGTCAGGCGGAGCGGGGCGCCCGTTCGGCCTGCTGTTCCCGAGCACGTCGTCG
TCGGGCGGCACGCCCGAGCGCACGGCGCCGGTGCGCGTCCCGCAGAAACGGACCTACCAGGCTGTGAGCCCCAAC
AAGAAGCACTGCGGCGCGGGCAGGAAGGCGAGCAAGGCCAAGCTCGCGTCCACAGCCCCAACCAAAGATCCCCAG
AGCCTCGCGGCCAAGCAGAACCGGCGCGAGCGGATCAGCGAGCGGCTGCGGGCGCTGCAGGAGCTGGTGCCCAAC
GGCACCAAGGTCGACCTGGTCACCATGCTCGAGAAGGCCATCAGCTACGTTAAGTTCCTCCAGTTGCAAGTCAAG
GTTCTGGCAACAGACGAATTCTGGCCGGCACAGGGAGGGAAGGCGCCGGAGATCTCCCAGGTGAGGGAGGCGCTC
GACGCCATCTTGTCGTCGGCGTCG
Zea mays ZmRSLb amino acid sequence (AZM4_70092: SEQ ID NO: 110)
MAQFLGAADDHCFTYEYEHVDESMEAIAALFLPTLDTDSANFSSSCFNYAVPPQCWPQPDHSSSVTSLLDPAENF
EFPVRDPLPPSGFDPHCAVAYLTEDSSPLHGKRSSVIEEEAANAAPAAKKRKAGAAMQGSKKSRKASKKDNIGDA
DDDGGYACVDTQSSSSCTSEDGNFEGNTNSSSKKTCARASRGAATEPQSLYARKRRERINERLRILQNLVPNGTK
VDISTMLEEAAQYVKFLQLQIKLLSCDDTWMYAPIAYNGINIGNVDLNIYSLQK*
Zea mays ZmRSLb nucleotide sequence (AZM4_70092: SEQ ID NO: 111)
ATGGCTCAGTTTCTTGGGGCGGCTGATGATCACTGCTTCACCTACGAGTATGAGCATGTGGATGAGTCCATGGAA
GCAATAGCAGCCCTGTTCTTGCCTACCCTTGACACCGACTCCGCCAACTTCTCCTCTAGCTGTTTCAACTATGCT
GTCCCTCCACAGTGCTGGCCTCAGCCAGACCATAGCTCTAGCGTTACCAGTTTGCTTGATCCAGCCGAGAACTTT
GAGTTTCCAGTCAGGGACCCGCTCCCCCCAAGCGGCTTCGATCCACATTGCGCTGTCGCCTACCTCACTGAGGAT
TCGAGCCCTCTGCATGGCAAACGTTCATCAGTCATTGAGGAAGAAGCAGCCAACGCCGCACCTGCTGCTAAGAAG
AGGAAGGCTGGTGCTGCAATGCAGGGATCAAAGAAATCCAGGAAGGCGAGCAAAAAGGATAACATCGGCGACGCC
GACGATGATGGCGGCTATGCCTGTGTTGACACGCAAAGCTCCAGTAGCTGCACCTCCGAGGACGGGAACTTCGAA
GGAAATACGAATTCAAGCTCCAAGAAGACCTGCGCCAGGGCCAGCCGCGGAGCAGCAACTGAACCTCAGAGTCTC
TATGCAAGGAAGAGGAGAGAGAGGATCAACGAAAGGTTGAGAATCTTGCAGAACTTGGTTCCAAATGGAACAAAA
GTAGACATTAGCACGATGCTCGAGGAAGCGGCGCAGTATGTCAAGTTTTTACAGCTCCAGATTAAGCTGTTGAGC
TGTGACGACACATGGATGTATGCGCCAATCGCGTACAATGGAATTAACATCGGCAATGTTGATCTGAACATCTAC
TCTCTGCAAAAGTAA
Zea mays ZmRSLc amino acid sequence (AZM4_91750: SEQ ID NO: 112)
MEDGGLXSEAGAWAELGTGGDESEELVAQLLGAFFRSHGEEGRHQLLWSDDQASSDDVHGDGSLAVPLAYDGCCG
YLSYSGSNSDELPLGSSSRAAPAGGPPEELLGAAETEYLNNVAAADHPFFKWCGNGEGLDGPTSVVGTLGLGSGR
KRARKKSGDEDEDPSTAIASGSGPTSCCTTSDSDSNASPLESADAGARRPKGNENARAAGRGAAAATTTTAEPQS
IYARVRRERINERLKVLQSLVPNGTKVDMSTMLEEAVHYVKFLQLQIRVLQLLSSDDTWMYAPIAYNGMGIGIDL
RMHGQDR*
Zea mays ZmRSLc nucleotide sequence (AZM4_91750: SEQ ID NO: 113)
ATGGAGGACGGAGGGTTGRTCAGCGAGGCCGGCGCCTGGGCCGAGCTCGGCACCGGCGGCGACGAGTCGGAGGAG
CTGGTGGCGCAGCTGCTGGGCGCCTTCTTCCGGTCCCACGGCGAGGAAGGCCGGCACCAGCTGCTTTGGTCTGAC
GACCAAGCTTCTTCCGACGACGTGCACGGCGACGGCAGCCTTGCCGTGCCGCTCGCATACGACGGCTGCTGCGGC
TATCTGAGCTACTCAGGTAGCAACTCGGACGAGCTCCCCCTCGGGAGCAGCTCCCGCGCTGCGCCAGCAGGTGGC
CCACCGGAGGAGCTGCTCGGTGCAGCTGAGACTGAGTACCTGAATAATGTGGCCGCCGCAGACCATCCCTTCTTC
AAATGGTGTGGGAATGGTGAGGGTCTGGATGGTCCGACGAGCGTCGTGGGCACGCTTGGGCTTGGCTCGGGCCGG
AAACGCGCGCGCAAGAAGAGCGGGGACGAAGACGAAGACCCGAGCACGGCCATCGCCAGCGGAAGCGGCCCCACG
AGCTGCTGCACTACCTCCGACAGCGACTCAAACGCGTCTCCTCTGGAGTCCGCGGACGCCGGCGCTCGTCGCCCC
AAGGGCAACGAGAATGCCCGGGCAGCTGGCCGCGGCGCGGCGGCGGCGACGACGACGACAGCGGAGCCCCAGAGC
ATCTACGCAAGGGTACGGAGGGAGCGGATCAACGAGAGGCTCAAGGTGCTGCAGAGCCTGGTGCCCAACGGCACC
AAGGTGGACATGAGCACCATGCTCGAGGAGGCCGTCCACTACGTCAAGTTCCTGCAGCTTCAGATCAGGGTGCTG
CAGCTCCTGAGCTCCGACGACACGTGGATGTACGCGCCCATCGCGTACAACGGGATGGGCATCGGGATCGACCTC
CGCATGCATGGACAGGACAGATGA
Zea mays amino acid sequence (AZM4_86104: SEQ ID NO: 114)
SKKSRKASKKDCIVDDDDVYVDPQSSGSCTSEEGNFEGNTYSSAKKTCTRASRGGATDPQSLYARKRRERINERL
RILQNLVPNGTKVDISTMLEEAAQYVKFLQLQIKLLSSDDMWMYAPIAYNGINISNVDLNIPALQK*
Zea mays ZmRSLd nucleotide sequence (AZM4_86104: SEQ ID NO: 115)
TCAAAGAAATCCAGGAAGGCGAGCAAAAAAGATTGTATTGTCGATGACGACGATGTCTATGTTGACCCGCAAAGC
TCCGGTAGCTGCACCTCCGAGGAGGGGAATTTTGAAGGGAATACGTATTCAAGCGCGAAAAAGACCTGCACCAGG
GCCAGCCGCGGAGGAGCAACTGATCCTCAGAGTCTCTATGCAAGGAAGAGGAGAGAGAGGATCAATGAAAGGTTG
AGAATCTTGCAGAACTTGGTCCCCAATGGAACAAAGGTTGACATTAGTACGATGCTCGAGGAAGCAGCACAGTAT
GTCAAATTTTTACAGCTTCAGATTAAGCTGTTGAGCTCTGACGACATGTGGATGTATGCGCCAATCGCGTACAAT
GGGATCAACATCAGCAATGTTGATCTGAACATCCCTGCA

1-56. (canceled)

57. A method of producing a plant with an altered root hair phenotype comprising:
incorporating a heterologous nucleic acid which encodes a RHD6-related polypeptide comprising an amino acid sequence having at least 50% sequence identity to an amino acid sequence selected from the group consisting of SEQ ID NOS: 13 to 25 into a plant cell by means of transformation, and;
regenerating the plant from one or more transformed cells.

58. The method according to claim 57 wherein the plant has increased tolerance to nutrient-deficient conditions relative to control plants or wherein the plant has increased production or secretion of a root-secreted phytochemical.

59. The method according to claim 57 wherein the RHD6-related polypeptide comprises an amino acid sequence having at least 55% sequence identity to SEQ ID NO:1 or SEQ ID NO:3.

60. The method according to claim 57 wherein the RHD6-related polypeptide comprises an amino acid sequence having at least 55% sequence identity to any one of SEQ ID NOS: 5, 7, 9, or 11, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, and 114.

61. The method according to claim 57 wherein expression is increased by expressing a heterologous nucleic acid encoding said RHD6-related polypeptide within cells of said plant.

62. The method according to claim 61 wherein the heterologous nucleic acid comprises a nucleotide sequence which has at least 40% sequence identity with any one of SEQ ID NO: 2 or SEQ ID NO: 4.

63. The method according to claim 61 wherein the heterologous nucleic acid comprises a nucleotide sequence which has at least 40% sequence identity with any one of SEQ ID NOS: 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115.

64. The method according to claim 61 wherein the heterologous nucleic acid is operably linked to a promoter.

65. The method according to claim 57 wherein expression is increased by a method comprising;
crossing a first and a second plant to produce a population of progeny plants;
determining the expression of the RHD6-related polypeptide in the progeny plants in the population, and
identifying a progeny plant in the population in which expression of the RHD6-related polypeptide is increased relative to controls.

66. The method according to claim 57 wherein expression is increased by a method comprising;
exposing a population of plants to a mutagen,
determining the expression of the RHD6-related polypeptide in one or more plants in said population, and
identifying a plant with increased expression of the RHD6-related polypeptide.

67. A plant produced by a method according to claim 57.

68. A method of modulating root hair development in a plant or increasing the tolerance of a plant to nutrient-deficient conditions comprising;
increasing the expression of a RHD6-related polypeptide comprising an amino acid sequence having at least 50% sequence identity to an amino acid sequence selected from the group consisting of SEQ ID NOS: 13 to 25 within cells of said plant relative to control plants.

69. A method of increasing the production or secretion of a root-secreted phytochemical in a plant comprising;
increasing the expression of a RHD6-related polypeptide within cells of a plant which secretes the phytochemical through its roots.

70. The method according to claim 68 wherein the RHD6-related polypeptide comprises an amino acid sequence having at least 55% sequence identity to SEQ ID NO:1 or SEQ ID NO:3.

71. The method according to claim 68 wherein the RHD6-related polypeptide comprises an amino acid sequence having at least 55% sequence identity to any one of SEQ ID NOS: 5, 7, 9, or 11, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, and 114.

72. The method according to claim 68 wherein expression is increased by expressing a heterologous nucleic acid encoding said RHD6-related polypeptide within cells of said plant.

73. The method according to claim 72 wherein the heterologous nucleic acid comprises a nucleotide sequence which has at least 40% sequence identity with any one of SEQ ID NO: 2 or SEQ ID NO: 4.

74. The method according to claim 72 wherein the heterologous nucleic acid comprises a nucleotide sequence which has at least 40% sequence identity with any one of SEQ ID NOS: 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115.

75. The method according to claim 69 wherein the RHD6-related polypeptide comprises an amino acid sequence having at least 55% sequence identity to SEQ ID NO:1 or SEQ ID NO:3.

76. The method according to claim 69 wherein the RHD6-related polypeptide comprises an amino acid sequence having at least 55% sequence identity to any one of SEQ ID NOS: 5, 7, 9, or 11, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, and 114.

77. The method according to claim 69 wherein expression is increased by expressing a heterologous nucleic acid encoding said RHD6-related polypeptide within cells of said plant.

78. The method according to claim 77 wherein the heterologous nucleic acid comprises a nucleotide sequence which has at least 40% sequence identity with any one of SEQ ID NO: 2 or SEQ ID NO: 4.

79. The method according to claim 77 wherein the heterologous nucleic acid comprises a nucleotide sequence which has at least 40% sequence identity with any one of SEQ ID NOS: 6, 8, 10, 12, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, and 115.

80. An isolated ROOT HAIR DEFECTIVE 6 (RHD6)-related gene selected from Root Hair Defective Six Like2 (RSL2), RSL3, RSL4, and RSL5.


SEQUENCE LISTING (228 sequences)

SEQ ID NO: 1 - protein, 298 aa, Arabidopsis thaliana
MALVNDHPNETNYLSKQNSSSSEDLSSPGLDQPDAAYAGGGGGGGSASSSSTMNSDHQQHQGFVFYPSGEDHHNSLMDFNGSSFLNFDHHESFPPPAISCGGSSGGGGFSFLEGNNMSYGFTNWNHQHHMDIISPRSTETPQGQKDWLYSDSTVVTTGSRNESLSPKSAGNKRSHTGESTQPSKKLSSGVTGKTKPKPTTSPKDPQSLAAKNRRERISERLKILQELVPNGTKVDLVTMLEKAISYVKFLQVQVKVLATDEFWPAQGGKAPDISQVKDAIDAILSSSQRDRNSNLITN

SEQ ID NO: 2 - DNA, 897 bp, Arabidopsis thaliana
atggcactcgttaatgaccatcccaacgagaccaattacttgtcaaaacaaaattcctcctcttccgaagatctctcctcgccgggactggatcagccagatgcagcttatgccggtggaggaggaggaggaggctcggcttcgagcagtagcacgatgaattcagatcatcaacaacatcaggggtttgtattttacccatccggtgaagatcatcacaactctttgatggatttcaacggatcatcatttcttaactttgatcatcacgagagctttcctcctccagccataagctgtggtggtagtagcggtgggggcggcttctccttcttggagggcaacaacatgagctacggcttcacaaactggaatcatcaacatcatatggatattattagccctagatccaccgaaactccccaaggccagaaagactggttatattctgattcaactgttgtaaccactggttctagaaacgagtctctttcgcctaaatccgctggaaacaaacgttctcacacgggagagagcactcaaccgtcgaagaaactgagtagcggtgtgaccggaaagaccaagcctaagccaacaacttcacctaaagatccacaaagcctagcagccaagaatcgaagagaaaggataagtgaacgtctcaagatattgcaagaacttgttcccaatggcaccaaggttgatttggtgacaatgcttgaaaaggctattagttatgtcaagttccttcaagtacaagttaaggtattagcgaccgatgagttttggccggctcaaggaggaaaagctcctgacatttctcaagttaaagacgccattgatgccattctctcctcatcacaacgagacaggaattcgaatctgatcaccaattaa

SEQ ID NO: 3 - protein, 307 aa, Arabidopsis thaliana
MSLINEHCNERNYISTPNSSEDLSSPQNCGLDEGASASSSSTINSDHQNNQGFVFYPSGETIEDHNSLMDFNASSFFTFDNHRSLISPVTNGGAFPVVDGNMSYSYDGWSHHQVDSISPRVIKTPNSFETTSSFGLTSNSMSKPATNHGNGDWLYSGSTIVNIGSRHESTSPKLAGNKRPFTGENTQLSKKPSSGTNGKIKPKATTSPKDPQSLAAKNRRERISERLKVLQELVPNGTKVDLVTMLEKAIGYVKFLQVQVKVLAADEFWPAQGGKAPDISQVKEAIDAILSSSQRDSNSTRETSIAE

SEQ ID NO: 4 - DNA, 924 bp, Arabidopsis thaliana
atgtcactcattaacgaacattgcaatgagcgtaattacatctcaaccccaaattcttcagaagatctctcttcaccacagaattgcggattagacgaaggagcttcagcttcaagcagtagcaccataaattctgatcatcaaaataatcaagggtttgtgttttacccttccggggaaaccattgaagatcataattctttgatggatttcaatgcttcatcattcttcacctttgataatcaccgaagccttatctctcccgtgaccaacggtggtgccttcccggtcgtggacgggaacatgagttacagctatgatggctggagtcatcatcaagtggatagtattagccctagagtcatcaaaactccaaatagctttgaaacaacgagcagttttggattgacttcaaactccatgagtaaaccggccacaaaccatggaaatggagactggttatactctggttcaactattgtaaacatcggttcaaggcacgagtccacgtcccctaaactggctggcaataaacggcctttcacgggagagaacacacaactttcaaagaagccgagtagcggtacgaatggaaagatcaagcctaaggcaacaacttcacctaaagatccacaaagcctagcagccaagaaccgaagagaaaggataagcgaacgcctcaaggtattgcaagaacttgtaccgaatggtaccaaggtggatttggtaactatgcttgagaaagcaattggctatgtaaagtttcttcaagtacaagttaaggtacttgcagccgatgagttttggccggcacaaggagggaaagctccggacatttctcaagttaaagaagctattgacgcaatcctctcatcatcacaacgagatagtaactcaactagagaaacaagtatagcagaataa

SEQ ID NO: 5 - protein, 352 aa, Arabidopsis thaliana
MEAMGEWSNNLGGMYTYATEEADFMNQLLASYDHPGTGSSSGAAASGDHQGLYWNLGSHHNHLSLVSEAGSFCFSQESSSYSAGNSGYYTVVPPTVEENQNETMDFGMEDVTINTNSYLVGEETSECDVEKYSSGKTLMPLETVVENHDDEESLLQSEISVTTTKSLTGSKKRSRATSTDKNKRARVNKRAQKNVEMSGDNNEGEEEEGETKLKKRKNGAMMSRQNSSTTFCTEEESNCADQDGGGEDSSSKEDDPSKALNLNGKTRASRGAATDPQSLYARKRRERINERLRILQNLVPNGTKVDISTMLEEAVHYVKFLQLQIKLLSSDDLWMYAPIAFNGMDIGLSSPR

SEQ ID NO: 6 - DNA, 1059 bp, Arabidopsis thaliana
atggaagccatgggagaatggagcaacaacctcggaggaatgtacacttatgcaaccgaggaagccgatttcatgaaccagcttctcgcctcttatgatcatcctggcaccggctcatcctccggcgcagcagccagtggtgaccaccaaggcttgtattggaaccttggttctcatcacaaccaccttagcctcgtgtctgaagccggtagcttctgtttctctcaagagagcagcagctacagcgctgggaacagcggatattacaccgttgttccacccacggttgaagagaaccaaaatgagacaatggactttgggatggaagatgtgaccatcaatacaaactcataccttgttggtgaggagacaagtgagtgtgacgttgagaaatactcttctggaaagactcttatgcctttggaaaccgtagtggagaaccacgatgacgaggaaagcttgttgcaatctgagatctctgtgactactacaaaatctctcaccggctccaaaaagagatcccgtgccacatctactgataaaaacaagagagcaagagtgaataagagggcccagaagaacgtagagatgagtggggataacaatgaaggagaagaggaagaaggagagacgaagttgaagaaaagaaagaatggggcaatgatgagtagacagaactcaagcaccactttctgtacggaggaagaatcaaactgcgctgatcaagacggtggaggagaagactcatcctctaaggaagatgatccctcaaaggccctcaacctcaatggtaaaacaagagccagtcgtggtgcagccaccgatcctcaaagcctctatgcaaggaaaagaagagaaaggattaacgagagactaaggattttacaaaatctcgtccccaatggaacaaaggtcgatattagtacaatgcttgaggaagcagttcattacgtcaaatttttgcagctccaaattaagttattgagctctgatgatctatggatgtatgcgccgattgctttcaatgggatggacattggtctcagctcaccgagatga

SEQ ID NO: 7 - protein, 519 aa, Arabidopsis thaliana
MEAMGEWSTGLGGIYTEEADFMNQLLASYEQPCGGSSSETTATLTAYHHQGSQWNGGFCFSQESSSYSGYCAAMPRQEEDNNGMEDATINTNLYLVGEETSECDATEYSGKSLLPLETVAENHDHSMLQPENSLTTTTDEKMFNQCESSKKRTRATTTDKNKRANKARRSQKCVEMSGENENSGEEEYTEKAAGKRKTKPLKPQKTCCSDDESNGGDTFLSKEDGEDSKALNLNGKTRASRGAATDPQSLYARVDISTMLEEAVQYVKFLQLQIKRLLAIGTNHRNRSIPLWTARNRQISKAHSRKRLRLRAVAKIIWSDEMTRFLLELITLEKQAGNYRGKSLIEKGKENVLVKFKKRFPITLNWNKVKNRLDTLKKQYEIYPAKLRSHPLRFIPLLDVVFRDETVVVEESWQPRRGVHRRAPVLDLSDSECPNNNGDEREDLMQNRERDHMRPPTPDWMSQTPMENSPTSANSDPPFASQERSSTHTQVKNVSRNRKRKQNPADSTLDRIAATMKKI

SEQ ID NO: 8 - DNA, 987 bp, Arabidopsis thaliana
atggaagccatgggagaatggagcaccggcctaggcggaatatatacagaggaagctgactttatgaatcagctccttgcctcctatgagcaaccttgtggcggttcatcttcagagacaaccgccacactcacggcctaccaccaccagggttctcaatggaatggtggcttttgcttctctcaggagagcagtagttatagtggttactgcgcggcgatgccacggcaagaagaagataacaatgggatggaggacgcgacaatcaacacgaacttgtaccttgttggtgaagagacaagtgaatgtgatgcgacggaatactccggtaaaagcctcttgcctttggagactgtcgcagaaaaccacgaccatagtatgctacagcctgagaactccttgaccacgaccactgatgagaaaatgttcaaccaatgtgagagttcaaagaagaggacgcgtgccacaacaactgataagaacaagagagccaacaaggcacgaaggagccagaaatgcgtagagatgagtggcgaaaatgaaaatagcggcgaagaagaatatacggagaaggctgcggggaagagaaagaccaaaccacttaagccgcaaaagacttgttgttcggatgacgaatcaaacggtggagacactttcttgtccaaagaagatggcgaggactctaaggctctcaacctcaacggcaagactagggccagccgcggcgcggccacagatcctcaaagcctttacgcaaggaaaagaagagagaggataaacgagaggctaaggattttgcaacatctcgtccctaatggaacaaaggttgatattagcacgatgttggaagaagcagtacaatacgtcaaatttctacagctccaaattaagttattgagctctgatgatctatggatgtatgcgcctattgcttacaacggaatggacattggccttgacctaaaactcaatgcactgaccagatga

SEQ ID NO: 9 - protein, 258 aa, Arabidopsis thaliana
MDVFVDGELESLLGMFNFDQCSSSKEERPRDELLGLSSLYNGHLHQHQHHNNVLSSDHHAFLLPDMFPFGAMPGGNLPAMLDSWDQSHHLQETSSLKRKLLDVENLCKTNSNCDVTRQELAKSKKKQRVSSESNTVDESNTNWVDGQSLSNSSDDEKASVTSVKGKTRATKGTATDPQSLYARKRREKINERLKTLQNLVPNGTKVDISTMLEEAVHYVKFLQLQIKLLSSDDLWMYAPLAYNGLDMGFHHNLLSRLM

SEQ ID NO: 10 - DNA, 777 bp, Arabidopsis thaliana
atggacgtttttgttgatggtgaattggagtctctcttggggatgttcaactttgatcaatgttcatcatctaaagaggagagaccgcgagacgagttgcttggcctctctagcctttacaatggtcatcttcatcaacatcaacaccataacaatgtcttatcttctgatcatcatgctttcttgctccctgatatgttcccatttggtgcaatgccgggaggaaatcttccggccatgcttgattcttgggatcaaagtcatcacctccaagaaacgtcttctcttaagaggaaactacttgacgtggagaatctatgcaaaactaactctaactgtgacgtcacaagacaagagcttgcgaaatccaagaaaaaacagagggtaagctcggaaagcaatacagttgacgagagcaacactaattgggtagatggtcagagtttaagcaacagttcagatgatgagaaagcttcggtcacaagtgttaaaggcaaaactagagccaccaaagggacagccactgatcctcaaagcctttatgctcggaaacgaagagagaagattaacgaaaggctcaagacactacaaaaccttgtgccaaacgggacaaaagtcgatataagcacgatgcttgaagaagcggtccattacgtgaagttcttgcagcttcagattaagttgttgagctcggatgatctatggatgtacgcaccattggcttacaacggcctggacatggggttccatcacaaccttttgtctcggcttatgtga

SEQ ID NO: 11 - protein, 223 aa, Arabidopsis thaliana
MENEAFVDGELESLLGMFNFDQCSSNESSFCNAPNETDVFSSDDFFPFGTILQSNYAAVLDGSNHQTNRNVDSRQDLLKPRKKQKLSSESNLVTEPKTAWRDGQSLSSYNSSDDEKALGLVSNTSKSLKRKAKANRGIASDPQSLYARKRRERINDRLKTLQSLVPNGTKVDISTMLEDAVHYVKFLQLQIKLLSSEDLWMYAPLAHNGLNMGLHHNLLSRLI

SEQ ID NO: 12 - DNA, 672 bp, Arabidopsis thaliana
atggagaatgaagcttttgtagatggtgaattggagtctcttttggggatgttcaactttgatcaatgttcatctaacgaatcgagcttttgcaatgctccaaatgagactgatgttttctcttctgatgatttcttcccatttggtacaattctgcaaagtaactatgcggccgttcttgatggttccaaccaccaaacgaaccgaaatgtcgactcaagacaagatctgttgaaaccaaggaagaagcaaaagttaagctcggaaagcaatttggttaccgagcctaagactgcttggagagatggtcaaagcctaagcagttataatagttcagatgatgaaaaggctttaggtttagtgtctaatacatcaaaaagcctaaaacgcaaagcgaaagccaacagagggatagcttccgatcctcagagcctatacgctaggaaacgaagagaaaggataaacgataggctaaagacattgcagagcctagttcctaatgggacaaaggtcgatataagcacaatgctggaagatgctgtccattacgtgaagttcctgcagcttcaaatcaagctcttgagttcagaagatctatggatgtatgcacctcttgctcacaatggtctgaatatgggactacatcacaatcttttgtctcggcttatttaa

SEQ ID NO: 13 - protein, 67 aa, Arabidopsis thaliana
TSPKDPQSLAAKNRRERISERLKILQELVPNGTKVDLVTMLEKAISYVKFLQVQVKVLATDEFWPAQ

SEQ ID NO: 14 - protein, 67 aa, Arabidopsis thaliana
TSPKDPQSLAAKNRRERISERLKVLQELVPNGTKVDLVTMLEKAIGYVKFLQVQVKVLAADEFWPAQ

SEQ ID NO: 15 - protein, 67 aa, Physcomitrella patens
GSANDPQSIAARVRRERISERLKVLQALIPNGDKVDMVTMLEKAISYVQCLEFQIKMLKNDSLWPKA

SEQ ID NO: 16 - protein, 67 aa, Physcomitrella patens
GSANDPQSIAARVRRERISERLKVLQALIPNGDKVDMVTMLEKAITYVQCLELQIKMLKNDSIWPKA

SEQ ID NO: 17 - protein, 67 aa, Physcomitrella patens
GSATDPQSVYARHRREKINERLKSLQNLVPNGAKVDIVTMLDEAIHYVKFLQNQVELLKSDELWIYA

SEQ ID NO: 18 - protein, 67 aa, Physcomitrella patens
GSATDPQSVYARHRREKINERLKNLQNLVPNGAKVDIVTMLDEAIHYVKFLQTQVELLKSDEFWMFA

SEQ ID NO: 19 - protein, 67 aa, Physcomitrella patens
GSATDPQSVYARHRREKINERLKTLQHLVPNGAKVDIVTMLDEAIHYVQFLQLQVTLLKSDEYWMYA

SEQ ID NO: 20 - protein, 67 aa, Physcomitrella patens
GSATDPQSVHARARREKIAERLRKLQHLIPNGGKVDIVTMLDEAVHYVQFLKRQVTLLKSDEYWMYA

SEQ ID NO: 21 - protein, 67 aa, Physcomitrella patens
GSATDPQSVYARHRREKINERLKTLQRLVPNGEQVDIVTMLEEAIHFVKFLEFQLELLRSDDRWMFA

SEQ ID NO: 22 - protein, 67 aa, Arabidopsis thaliana
GAATDPQSLYARKRRERINERLRILQNLVPNGTKVDISTMLEEAVHYVKFLQLQIKLLSSDDLWMYA

SEQ ID NO: 23 - protein, 67 aa, Arabidopsis thaliana
GAATDPQSLYARKRRERINERLRILQHLVPNGTKVDISTMLEEAVQYVKFLQLQIKLLSSDDLWMYA

SEQ ID NO: 24 - protein, 67 aa, Arabidopsis thaliana
GTATDPQSLYARKRREKINERLKTLQNLVPNGTKVDISTMLEEAVHYVKFLQLQIKLLSSDDLWMYA

SEQ ID NO: 25 - protein, 67 aa, Arabidopsis thaliana
GIASDPQSLYARKRRERINDRLKTLQSLVPNGTKVDISTMLEDAVHYVKFLQLQIKLLSSEDLWMYA

SEQ ID NO: 26 - protein, 762 aa, Physcomitrella patens
MAGPAGALWSTCDPQPIQQAEIFSGPDNQAGLMSFHVDTPFHWGSEPWALHSRSDDIALMSPSLVHDISPYDSVLHLSGVSGDVQDLVCGNPKFRQSGQWGQSEFSYSVQDNMQDLLTNQFIPYNTSSLGLNHLSPNFTDLDCAPVYNDTKAFGTVTHNRAVPSTNTQSAQHGSSSMVSSNRPITSTASPTTQYGGPRTPSQTTQYGGSSMVTNSMEMFASAAPQGIMTTSGLSGGCNSDLMHLPKRQHAHSLPPTTGRDLTASEVVSGNSISNISGVGSFNSSQKSSASVMMSPLAASSHMHKAAAVSEELKMASFNPGPFVPTQKKQQHEQQDTMTSNRIWADKNNLGKISSSPIPIMGFEQSQQQSMSNSSPVTSLGFEQRQKMSMGSSPSITIIGFEQRQKQPMSSSSPISNMVFEPRQKQPMSSSSPISNIVFEQRQLPTVGSSPPISISGFEPKKQPSLSNSPPLSNLGFEQRLQPMSNASPISNLPFEQQRQQATMSNTRSAEPDSVESTTKWPLRMDGAIGGCAGLPSSQKAPVIMQPETGTMKCPIPRTMPSNAKACPAVQNANSVNKRPLTVDDKDQTGSMNKKSMQKFLGPQGCSRLESISALAHQKVSQSTTSGRALGPALNTNLKPRARQGSANDPQSIAARVRRERISERLKVLQALIPNGDKVDMVTMLEKAISYVQCLEFQIKMLKNDSLWPKALGPLPNTLQELLELAGPEFAGIDGKNTEESSEKPKKSALEVIELDGNQPSAD

SEQ ID NO: 27 - DNA, 2289 bp, Physcomitrella patens
atggcaggtccagcaggagctttatggagtacttgtgatccacagcctattcaacaggcagagatatttagtggtcctgacaaccaagctggtttgatgtcttttcatgtggataccccgttccattggggatctgaaccatgggctctccactctcggtcagatgacatcgccttgatgtccccctcgcttgttcacgacatatcaccttatgattctgtcttgcatctttccggagtgtctggggatgtgcaagatttagtttgcgggaatcccaaatttcgccaaagtgggcaatgggggcagagcgagttttcatactctgttcaggacaacatgcaagatctcctaaccaaccagttcataccgtacaacacatcttcattgggtttaaatcatctctccccgaatttcaccgacttggattgcgcaccggtatacaatgataccaaggcttttggcactgttacacacaacagggcagtcccgagcactaatacccagagtgctcagcacgggagttcgtctatggtttcaagtaacaggccaatcactagcacagcttctcctactactcagtatggaggtccgaggactccatcccaaaccacccagtacgggggttcatctatggttaccaactcgatggaaatgtttgcttcagctgcacctcagggtattatgactacatctggcttgagtggcggttgcaactcagacttgatgcatctgccgaagcgccagcatgctcactctcttcctcctaccactggcagagatttaactgcatctgaagtggtatctggaaattcgatatcaaacatttccggggttggatcttttaacagcagccagaaaagcagtgcatccgtgatgatgtctcctttagctgcttcttctcacatgcacaaggctgctgctgtatctgaagaacttaagatggcaagtttcaaccctggtccattcgtacctacgcagaaaaagcagcaacatgagcagcaggatacgatgacctctaatcgtatatgggcggataagaacaacttgggaaaaattagttcatcgcccattccgatcatggggtttgagcagagtcaacagcaatccatgagcaattcctcccctgttaccagtttggggtttgagcaaaggcaaaaaatgtccatgggtagctctccctccatcacgatcattggatttgagcaaagacagaagcaacctatgagtagttcttcccccatttcaaacatggtttttgaaccaagacaaaaacagccaatgagtagctcttctcctatctctaatattgtctttgagcaaagacaactcccaactgtgggtagctctcctccgatttcaatctcaggatttgagccaaagaaacaaccatctttgagcaattctcctcccctctctaatctgggttttgagcaaaggctacaacccatgagtaatgcatctcctatttccaacttaccctttgagcaacaaagacaacaagcaaccatgagtaacaccagatctgcagaacccgattctgtcgagtctaccacgaagtggcccttgcggatggatggtgccataggtggatgtgctggcttaccaagcagtcagaaagctcctgttatcatgcagcctgagactgggactatgaagtgtcctattccgaggaccatgcccagcaatgctaaggcttgcccagctgtgcagaatgctaattccgtaaacaagcgccctcttacggttgatgacaaggaccaaactggatcgatgaataagaagtcgatgcaaaagtttttgggacctcaaggttgtagcagacttgaaagtatcagtgctttagctcaccaaaaagtgagtcaaagtacaacaagcggtcgtgctctagggcctgctttgaacaccaatctcaagcctcgtgcacgccaagggagtgccaatgatccgcagagcattgctgctagggtgcgaagagaaagaataagtgagcggctcaaagttttgcaagccttgatacctaacggtgataaagtggatatggtcaccatgctggagaaggctatcagctacgtgcagtgtttggaatttcagattaagatgttaaaaaatgactctttgtggcctaaggcgcttggccctctaccgaacactttgcaagagcttctcgaacttgctgggccagagtttgccggcatagatggcaagaatactgaggagtcgtcagagaaaccgaagaaatctgctcttgaagtaattgagttggacggcaatcagccttctgctgactaa

SEQ ID NO: 28 - protein, 173 aa, Physcomitrella patens
MNKKPMQKALGPQGCSRLESISALAHQKVSQSASGRALGPALNTNLKPRARQGSANDPQSIAARVRRERISERLKVLQALIPNGDKVDMVTMLEKAITYVQCLELQIKMLKNDSIWPKALGPLPNTLQELLELAGPEFSGTESKNVEEPPAKPKKSAPDVIEFDGNQPSADKE

SEQ ID NO: 29 - DNA, 522 bp, Physcomitrella patens
atgaataagaagcctatgcaaaaagctttgggacctcaaggatgcagcaggctagaaagcatcagtgctttagctcatcaaaaagtgagtcagagtgcaagtggtcgtgcactagggcctgctctgaacaccaacctcaagcctcgtgctcgtcaagggagtgccaatgacccacagagcattgccgctagggttcgaagagaaaggataagtgagcggctgaaagttttgcaagccttgatacctaatggtgataaggtagatatggtgaccatgctggagaaggctatcacctacgtgcagtgtctggaactccagattaagatgttaaagaatgattctatctggcccaaggcgcttggacctctaccaaacactcttcaagagcttctggagcttgctggaccagaattttctggaacggaaagcaagaatgtagaggagcccccagcgaagccaaagaaatcagctcctgacgttattgagttcgacggcaatcaaccttctgccgacaaagagtag

SEQ ID NO: 30 - protein, 67 aa, Physcomitrella patens; identical to SEQ ID NO: 19

SEQ ID NO: 31 - DNA, 201 bp, Physcomitrella patens
ggttcagcgactgatccgcagagtgtatatgccaggcatagaagggagaagatcaacgagcgcttgaagacattacagcacttggtaccaaatggagctaaggtagacatcgtgaccatgcttgacgaagccattcactacgtccaatttctgcagctccaagtgacgctgttgaagtcggatgaatattggatgtacgcc

SEQ ID NO: 32 - protein, 538 aa, Physcomitrella patens
MTDLISILESSGSSREEMCPVAVPSSVASSCERLIWEGWTAQPSPVEESTTSKLLPKLLPELETSSYSALTLQQPDALSSILSVLHPFSHYSSASLELARNPDWSLKSSNPLRESSSEAGIRTSSFEGLYSGQHTTKKIHLGVIPYHLSEDQRQCAVSPPENECRLLSANSSGSLHWWHSIGPESPSSTLAFHNIGIQHSTFEKCEPRGQSHSSWPAASGTSPTVQYFHAHSADNEGVEVVKQDDSQISKALATYQPHGDHSLVLNSDRIASTTSHSEDPCGPKPGRRPAASYDTEMILSPSESFLTTPNMLSTLECVISGASNISDQYMNFVREPQEQRLSSISDLSLIPDSHADPHSIGFISGTFRTDSHGTGIRKNRIFLSDEESDFLPKKRSKYTVRGDFQMDRFDAVWGNTGLRGSSCPGNSVSQMMAIYEFGPALNRNGRPRVQRGSATDPQSVHARARREKIAERLRKLQHLIPNGGKVDIVTMLDEAVHYVQFLKRQVTLLKSDEYWMYATPTSYRSKFDDCSLVPGENN

SEQ ID NO: 33 - DNA, 1617 bp, Physcomitrella patens
atgaccgatctgatttcgatcttggagtcatcagggtcatcacgagaggagatgtgccctgttgctgtgccaagctccgtggcttcttcttgtgaaaggttgatatgggaggggtggactgcacaaccatctcctgtcgaagaaagcaccaccagcaagttacttccaaagctacttccagagctcgagacatcatcctactctgcactcacccttcagcaacctgatgcgctctccagcatactttcagtcctccaccctttttctcattacagttcggccagtttagaactcgctcgcaatcctgactggagcttgaaatcttcaaatcctctgcgggaaagcagctcggaggctggcatccgaacctcatctttcgaaggcttgtactctggtcagcacaccaccaaaaagattcatttgggggtcataccctaccacttgtccgaagatcagcgccagtgcgctgtcagtcctccggaaaatgagtgccgcctactgtctgcaaattcctctggatcccttcactggtggcattccataggccccgagtctccttcctctactcttgcattccataatattgggatccaacactctaccttcgaaaagtgtgagcctaggggccagtcgcactcatcatggccagcggccagcggcacgtcgccaacagttcaatactttcatgcccattctgcagataatgaaggtgtcgaggtcgtcaagcaagatgactcgcagatatccaaggctctggcgacctatcaaccccacggcgaccatagtctcgtgctaaattcagaccgcattgcaagcacaaccagccactcagaagatccttgcggccctaaacctggacgcagaccagctgcatcatacgacaccgagatgattcttagcccaagtgagagtttcttgacaactcccaatatgttatcaacgttggagtgcgtaatatccggtgcaagtaacatatctgatcagtatatgaacttcgtcagagaaccgcaggagcaaaggctgtcctctatctccgatctgtcccttattcctgacagccacgcggatccgcacagtatcggatttatctctgggacctttagaacagactcccacggaactggaataagaaagaaccgcatctttctcagtgatgaggaatccgacttcttgcctaagaagcgatccaagtacacggtccgcggcgattttcagatggatcgcttcgacgcagtttgggggaataccggtcttcggggatctagctgtcctggaaattcagtatcccagatgatggcgatttacgaattcggacccgcactgaacaggaacggcaggccgcgagtacaacgtggttcggcgactgatccgcagagtgtacacgccagggcgcggagggagaaaatcgccgagcgcttgagaaagttgcagcacctcattccaaacggcgggaaggtggacatcgtaaccatgctcgacgaagccgttcactatgttcagtttttgaagcgacaagttacgcttctgaaatccgacgagtattggatgtacgccacgccgacctcgtaccggagcaaattcgacgactgcagtctggttcccggcgagaacaactga

SEQ ID NO: 34 - protein, 539 aa, Physcomitrella patens
MVQLYMSSVEEQRETMVQPYVSSMDSGSTSGRQTPSCVVQQGSNTFETSNLWEEWTQASNGDDTVSTSNFLPEISSFTSSRLSFQQSDSLTTWMSGFPPLSQTALSPDLSHSSDPVDHPPAFMQEGLGPGDSILDYSPALTEMYPKSSSKHNSSDCLPYPAASAPDKKMTDHELGSAISLAYDRGTVSRQLLRALGPLSPSSPLALQNGLQNPLGDPWDASPSAMPWPMATTGHAYGPGATRTSIPDHLANAINHLEGIAPSSASHASKPRHTDIFIAPNGTFDSTPGGWTPQYYDGSVTTDESVKAMKLIASLREAGHAEATIGFCTESKPSFLRGGDRTTSPVDSFFGKCVGAKTSIKQACSGKHPLELEEIVDSENSELNPTQLKRSKLFENHPNALWSDQSMNGRELRSYSHLVGSSLTASQPMDIIAIGPALNTDGKPRAKRGSATDPQSVYARHRREKINERLKSLQNLVPNGAKVDIVTMLDEAIHYVKFLQNQVELLKSDELWIYATPNKYNGMDISDLSDMYLQELESRA

SEQ ID NO: 35 - DNA, 1620 bp, Physcomitrella patens
atggtgcagttatacatgtcctcagttgaagagcagcgggaaacaatggtacagccatacgtctcaagcatggactcaggctcaacgtcggggcgccagacgccatcttgcgtcgttcagcagggaagtaacacatttgagacttcgaatctgtgggaggaatggacgcaagcatcgaacggcgacgatacagtctccaccagcaatttcctccccgaaatcagttccttcacgtcgagtcgtctctccttccagcaaagcgactctctcaccacttggatgtcagggttccctcccctctcccaaactgccttgagcccggatcttagtcactcctccgaccccgtggatcatcccccagcattcatgcaggagggtttaggccccggtgattctattctggactattcccccgctctcacagagatgtacccgaaaagtagctccaaacataattcctcggattgtttaccttaccctgcggccagtgcaccagacaaaaaaatgactgatcacgaactaggttcggctatttccctcgcgtatgatagaggcaccgtttcccgccagcttcttcgagccttgggcccattgtcgccttcatcgcctctagcattgcagaatgggctgcaaaacccgcttggggacccctgggatgcttctccatctgcaatgccgtggccaatggcaacaaccggtcatgcttatggaccaggcgccaccaggacttctattccagatcacttagcaaatgcaattaatcacctggagggcattgcaccgtccagtgccagtcatgcatcgaaacctcgtcacactgatattttcattgcacccaatggcacgttcgattcgacgccgggaggttggacaccgcagtattacgatgggtccgtgacgacagatgagtctgtgaaggcgatgaagctgattgcgtccctacgtgaagcaggccacgcagaggctacaattggattctgtacagagagcaagcctagttttctcaggggtggggacagaacaacctcgccagtggacagcttcttcggcaaatgtgtaggggccaaaacgagtataaagcaagcctgttctgggaaacaccctcttgaacttgaggagatcgttgatagtgaaaacagtgaattaaatcccacccagctcaaacgctctaaactttttgagaatcatccgaatgccttgtggagcgatcagagtatgaatggaagagaactgagatcgtactctcatttggttggcagcagtcttactgcatcgcagcccatggacataattgcaattggcccagcgctcaacactgatggcaaaccacgagcaaagcggggttcagcaaccgatcctcagagtgtttacgctagacataggagagaaaaaatcaacgaacgattgaagagtttacaaaacctagtacctaatggagccaaggttgacatagtaaccatgctggacgaagctatacattacgtcaaatttttacaaaatcaagttgagctgctgaagtccgacgagttgtggatttacgcaacaccaaataagtacaacggcatggacatttccgacctctctgacatgtatttgcaggagctggagtcacgtgcgtga

SEQ ID NO: 36 - protein, 538 aa, Physcomitrella patens
MVRFNYMYPVQEQLEAMTDQHTPSMDSVSSAGEKTSSCIVQQGGNASETSNLWEEWTQGSNGDDSVSTSNFLPELNSSTSSRLAFHQSDILSTWISGYHPLSQSSLSSEFSHTSDRENHPPAFMQEGLIPSGLILDSDPALTDIYTRSSSSDSLPYPTARIMDKALTDHELESAVPLAYEKGCVPPQVLRNLGPLSPSSPLAFQNGLLNPLRDPWDSCPSALPWSNVTTASQTYGQVTTRTFIPDHSASAIDKLEAVATITAGYGASKPQHTDVFIEPNGTFQSTPAGWAPQFYDGSEATGLLVKPMRAIASLGEAGCGEATSEFCTKTKPGLLKGGDTITSPVGSLLGDCKKAESSMKQVWPGKHRLELVELVDGEDTKSSPTQLKRPKHSTDYANVLLSDHILKGAELRSYFHSGDVGLNASQAMDIIVIGPALNTNGKPRAKRGSATDPQSVYARHRREKINERLKNLQNLVPNGAKVDIVTMLDEAIHYVKFLQTQVELLKSDEFWMFANPHNYNGIDISDPSSMHSPELESNI

SEQ ID NO: 37 - DNA, 1617 bp, Physcomitrella patens
atggtgcggtttaactacatgtacccggttcaagagcagctggaagccatgacggaccaacacaccccaagcatggattcggtctcgtcggccggagagaagacatcctcttgcatcgtccagcagggaggaaatgcatccgaaacttcaaacttgtgggaagaatggacacaagggtcgaacggcgacgattctgtctctaccagcaacttcctccccgaactgaattcctccacctccagtcgtctcgcattccaccaaagcgacattctttccacttggatctcaggctaccacccactctcgcaaagcagcctgagttccgaattcagccacacctccgaccgcgagaatcaccccccagcattcatgcaagagggtttaatccccagtggtttaattcttgactctgatcctgctctcacagatatttatacgagaagcagctcctcggactctttgccataccccacggctaggatcatggacaaagcattgaccgatcacgagcttgagtctgctgtcccacttgcatatgaaaaaggctgcgttcctccccaggttctgcgtaacctagggccattgtcaccttcttcgcctctggcattccagaatggactgctaaaccccctcagggacccttgggattcgtgtccatctgcattgccatggtcaaatgtgaccacagccagccagacttacggtcaagtgacaaccaggactttcattccagatcactctgcaagtgcaatcgacaagttggaggccgtcgcaacgatcactgccggatacggcgcgtcgaaaccacaacatactgacgtcttcatagaacccaacgggacgtttcagtcgactccggcagggtgggcaccgcagttttacgatggatccgaggcgacgggcctgttggtcaagccaatgagggccatcgcatctctgggtgaagccggctgtggggaggccactagtgaattctgcacaaagaccaagccaggacttctcaaaggtggggacacaataacctcgccggtgggtagcctgttgggcgattgcaaaaaagctgagtcaagtatgaagcaagtttggcctggaaaacaccgtcttgaactcgtggaactagtcgatggtgaagacaccaaatcaagtcccacccagctcaaacggccgaaacattctacggattatgcgaatgtcctgttgagcgatcatattctgaaaggagcggagctgcggtcctacttccattctggtgatgttggtctaaatgcatctcaagcgatggacattattgtaattggcccagccttgaatactaatggcaagccgcgagctaaacggggttcagccaccgatccccagagtgtgtacgctagacataggcgagaaaaaatcaacgaacgactgaagaatttacaaaatctcgtgccaaatggagccaaggttgacattgtgaccatgctagacgaagccatacactacgtcaaattcttgcaaactcaagttgagctgctgaaatccgacgagttctggatgttcgcaaatccacacaactacaacggcatagatatctccgatccctctagcatgcattcgccggagctggagtcgaatatttag

SEQ ID NO: 38 - protein, 67 aa, Physcomitrella patens; identical to SEQ ID NO: 21

SEQ ID NO: 39 - DNA, 201 bp, Physcomitrella patens
gggtcagctactgatcctcagagtgtgtacgcaaggcatcgccgggagaagattaacgagcgcctaaagacattgcagcggttggttcctaacggagaacaggtcgacattgtgaccatgctggaagaagccattcactttgtcaaatttttggagttccaactggagctgttgcgatccgatgatcgctggatgttcgcc

SEQ ID NO: 40 - protein, 80 aa, Selaginella moelendorfii
LNTNLKPRAKQGCANDPQSIAARQRRERISDRLKILQELIPNGSKVDLVTMLEKAINYVKFLQLQVKVLMNDEYWPPKGD

SEQ ID NO: 41 - DNA, 240 bp, Selaginella moelendorfii
ctcaacactaatcttaagccgcgagcaaagcaaggttgtgctaatgatccacaaagcattgctgccagacaacgaagagaacggataagtgaccggcttaaaatcctgcaggagctcataccaaatggatccaaggtcgatctggtaaccatgctggagaaggccatcaactacgtcaagttcttgcaattgcaagtcaaagttcttatgaacgatgagtattggccaccaaagggagat

SEQ ID NO: 42 - protein, 80 aa, Selaginella moelendorfii; identical to SEQ ID NO: 40

SEQ ID NO: 43 - DNA, 240 bp, Selaginella moelendorfii
ctcaacactaatcttaagccgcgagcaaagcaaggttgtgctaatgatccacaaagcattgctgccagacaacgaagagaacggataagtgaccggcttaaaatcctgcaggagctcataccaaatggatccaaggtcgatctggtaaccatgttggagaaggccatcaactacgtcaagttcttgcaattgcaagtcaaagttcttatgaacgatgagtattggccaccaaagggagat

SEQ ID NO: 44 - protein, 76 aa, Selaginella moelendorfii
LNTNFKPRARQGSANDPQSIAARHRRERISDRLKILQELVPNSTKVDLVTMLEKAINYVKFLQLQVKVLTSDDYWP

SEQ ID NO: 45 - DNA, 228 bp, Selaginella moelendorfii
ctcaacaccaatttcaagcctcgagccaggcagggaagcgccaatgatccccagagcatcgctgctagacatcgccgggagaggatcagtgacaggctcaagatcttgcaagagctcgttccaaacagcacaaaggttgatctagtgacgatgctggagaaggccatcaattacgtcaagttcctccagctgcaagttaaggtgcttacgtcggacgactactggcca

SEQ ID NO: 46 - protein, 76 aa, Selaginella moelendorfii; identical to SEQ ID NO: 44

SEQ ID NO: 47 - DNA, 228 bp, Selaginella moelendorfii
ctcaacaccaatttcaagcctcgagccaggcagggaagcgccaatgatccccagagcatcgctgctagacatcgccgggagaggatcagtgacaggctcaagatcttgcaagagctcgttccaaacagcacaaaggttgatctagtgacgatgctggagaaggccatcaattacgtcaagttcctccagctgcaagttaaggtgcttacgtcggacgactattggcca

SEQ ID NO: 48 - protein, 80 aa, Selaginella moelendorfii
LNTDGKPRAKRGSATDPQSIYARQRRERINERLRALQGLVPNGAKVDIVTMLEEAINYVKFLQLQVKLLSSDEYWMYAPT

SEQ ID NO: 49 - DNA, 240 bp, Selaginella moelendorfii
ctaaacaccgacggaaagccacgcgcaaagcgtggatctgccacggacccgcaaagcatctacgctcggcaaagaagagaaaggatcaacgagcgtttgagagcgctacaaggactcgtaccaaacggagcgaaggttgacattgtgacgatgctcgaggaagccatcaactatgtcaagtttttgcagctgcaagtaaagctgctcagctcggacgagtattggatgtacgcccccaca

SEQ ID NO: 50 - protein, 80 aa, Selaginella moelendorfii
LNTNGKPRAKRGSATDPQSVYARHRRERINERLKTLQHLVPNGAKVDIVTMLEEAIHYVKFLQLQVNMLSSDEYWIYAPT

SEQ ID NO: 51 - DNA, 240 bp, Selaginella moelendorfii
ctcaacacgaatggcaagcccagagcaaagcgtggatctgcaacagatccccaaagcgtttacgcaaggcaccggagagagaggatcaacgagaggctcaaaactttacaacaccttgttccaaatggtgcaaaggttgacatagtgacaatgcttgaagaagcaatacattacgtgaagtttctacagctgcaagtcaacatgttaagctctgatgagtactggatttatgcacccaca

SEQ ID NO: 52 - protein, 80 aa, Selaginella moelendorfii
LNTNGKPRAKRGSATDPQSVYARHRRERINERLKTLQHLVPNGAKVDIVTMLEEAIHYVKFLQLQVNMLSSDEYWTYAPT

SEQ ID NO: 53 - DNA, 240 bp, Selaginella moelendorfii
ctcaacacgaatggcaagccccgagcaaagcgtggatctgcaacagatccccaaagcgtttatgcaaggcaccggagagagaggatcaacgagaggctcaaaactttacaacaccttgttccaaatggtgcaaaggttgacattgtgacaatgcttgaagaagcaatacattacgtgaagtttctacagctgcaagtcaacatgttaagctctgatgagtactggacttatgcacccaca

SEQ ID NO: 54 - protein, 80 aa, Selaginella moelendorfii; identical to SEQ ID NO: 48

SEQ ID NO: 55 - DNA, 240 bp, Selaginella moelendorfii
ctaaacaccgacggaaagccacgcgcaaagcgtggatctgccacggacccgcaaagtatctacgctcggcaaagaagagaaaggatcaacgagcgtttgagagcgctacaaggactcgtaccaaacggagcgaaggttgacattgtgacgatgctcgaggaagccatcaactatgtcaagtttttgcagctgcaagtaaagctgctcagctcggacgagtattggatgtacgcccccaca

SEQ ID NO: 56 - protein, 295 aa, Oryza sativa
MMAAQASSKRGMLLPREAVLYDDEPSMPLEILGYHGNGVGGGGCVDADYYYSWSGSSSSSSSSVLSFDQAAVGGSGGGCARQLAFHPGGDDDDCAMWMDAAAGAMVENTSVVAGGGNNYCHRLQFHGGAAGFGLASPGSSVVDNGLEIHESNVSKPPPPAAKKRACPSGEARAAGKKQCRKGSKPNKAASASSPSPSPSPSPSPNKEQPQSAAAKVRRERISERLKVLQDLVPNGTKVDLVTMLEKAINYVKFLQLQVKVLATDEFWPAQGGKAPELSQVKDALDAILSSQHPNK

SEQ ID NO: 57 - DNA, 888 bp, Oryza sativa
atgatggcagctcaggcaagcagcaagcgcggcatgctgctgccacgggaggcggtgctctacgacgacgagccctccatgccgctggagatcttgggctaccacggcaatggcgtcggcggcggtggctgcgttgacgccgattactactacagctggtcggggtccagctccagctccagctcgtcggtgctcagctttgaccaggcggcggtcggcggcagcggcggcggctgcgcccggcagctggctttccatcccggcggcgacgacgacgactgcgccatgtggatggacgccgccgccggcgccatggtcgagaacacgtctgtcgtcgccggcggcggcaacaactactgtcatcgcctgcagttccacggcggcgccgccggtttcggactcgcgagcccaggctcgtcggtcgttgacaacggcctcgaaatccacgagagcaacgtcagcaagccgccaccgccggcagccaagaagcgcgcatgcccgagcggcgaggcgagagcagcggggaagaagcagtgcaggaaagggagcaagccaaacaaggctgcttctgcttcttctccttctccttctccttctccttctccttctcctaacaaggaacaacctcaaagcgccgctgcaaaggtaagaagagagcggatcagtgagaggctcaaagttcttcaggatctcgtgcctaatggcacaaaggtagacttggtcaccatgctagaaaaggcgatcaactacgtcaaattcctccagctgcaagtgaaggttttggctactgatgagttctggccggcacaaggagggaaagcaccagagctctctcaagtcaaggacgccttggacgccatcctatcttctcagcatccaaacaaatga

SEQ ID NO: 58 - protein, 300 aa, Oryza sativa
MRMALVRERAMVYGGGCDAEAFGGGFESSQMGYGHDALLDIDAAALFGGYEAAASAGCALVQDGAAGWAGAGASSSVLAFDRAAQAEEAECDAWIEAMDQSYGAGGEAAPYRSTTAVAFDAATGCFSLTERATGGGGGAGGRQFGLLFPSTSGGGVSPERAAPAPAPRGSQKRAHAESSQAMSPSKKQCGAGRKAGKAKSAPTTPTKDPQSLAAKNRRERISERLRILQELVPNGTKVDLVTMLEKAISYVKFLQLQVKVLATDEFWPAQGGKAPEISQVKEALDAILSSSSPLMGQLMN

SEQ ID NO: 59 - DNA, 903 bp, Oryza sativa
atgcgcatggcgctggtgcgggagcgcgcgatggtgtacggtggagggtgcgacgccgaggcgttcggcggcgggttcgagtcgtcccagatggggtacggccacgacgcgctgctcgacatcgacgcggcggcgctgttcggggggtacgaggcggccgccagcgccgggtgcgccctcgtgcaggacggcgccgcggggtgggcgggcgcgggcgcgtcgtcctcggtgctggcgttcgaccgcgccgctcaggcggaggaggccgagtgcgacgcgtggatcgaagccatggaccagagctacggcgccggcggcgaggcggcgccgtaccggtcgacgacggccgtcgccttcgacgcggccaccggctgcttcagcctgacggagagagccaccggcggcggcggcggcgcgggtgggcggcagttcgggctgctgttcccgagcacgtcgggcggcggcgtctcccccgaacgcgccgcgccggcgccggcgccccgcggctcgcagaagcgggcccacgcggagtcgtcgcaggccatgagccctagcaagaagcagtgcggcgccggcaggaaggcgggcaaggccaagtcggcgccgaccaccccaaccaaggacccgcaaagcctcgcggccaagaatcggcgcgagaggatcagcgagcggctgcggatcctgcaggagctcgtgcccaacggcaccaaggtcgacctcgtcaccatgctcgagaaggccatcagctacgtcaagttcctccagcttcaagtcaaggttcttgcgacggacgagttctggccggcgcagggagggaaggcgccggagatatcccaggtgaaggaggcgctcgacgccatcttgtcgtcgtcgtcgccgctgatgggacaactcatgaactga

SEQ ID NO: 60 - protein, 294 aa, Oryza sativa
MAMVAGDEAMSVPWHDVGVVVDPEAAGTAPFDAGAGYVPSYGQCQYYYYYDDHHHHPCSTELIHAGDAGSAVAVAYDGVDGWVHAAAAATSPSSSSALTFDGHGAEEHSAVSWMDMDMDAHGAAPPLIGYGPTAATSSPSSCFSSGGSGDSGMVMVTTTTPRSAAASGSQRRARPPPSPLQGSELHEYSKKQRANNKETQSSAAKSRRERISERLRALQELVPSGGKVDMVTMLDRAISYVKFMQMQLRVLETDAFWPASDGATPDISRVKDALDAIILSSSSPSQKASPPRSG

SEQ ID NO: 61 - DNA, 885 bp, Oryza sativa
atggctatggtggccggcgacgaggcgatgtcagtgccatggcacgacgtcggcgtcgtcgtcgaccccgaggcggccgggacggcgccgttcgacgccggcgccggctatgtcccatcgtacggtcagtgccaatactactactactacgacgaccaccaccaccacccgtgcagcacggagctgatccacgcgggcgacgctggcagtgcggttgcggttgcgtacgacggcgtcgacggctgggttcacgccgccgccgcagccacctccccgtcctcgtcatctgcgctcaccttcgatggtcacggcgccgaggagcacagcgcagtgtcgtggatggacatggacatggacgcgcacggcgccgcgcctcccctaatcggctacggcccgacggcggcgacctcctccccctcctcctgcttcagctccggcggctccggcgacagcggcatggtgatggtgaccaccaccaccccgaggagcgccgccgcctctggttcgcagaggcgggcacgcccgccgccgtcgccgttgcagggatcagagctgcacgagtactccaagaagcagcgcgccaacaacaaggagacacagagctcagctgccaagagccggcgggagaggatcagcgagcggctgagggcgctgcaggagctggtgccgagcggcgggaaggtggacatggtgaccatgctggacagggccatcagctacgtcaagttcatgcagatgcagctcagggtgctggagaccgacgcgttctggccggcgtccgacggcgccacgccggacatctcccgggtcaaggacgcgctcgacgccatcatcctctcctcgtcctcgccctcgcaaaaggcttctcctcctcggtcgggctag

SEQ ID NO: 62 - protein, 324 aa, Oryza sativa
MEDSEAMAQLLGVQYFGNDQEQQQPAAAAPPAMYWPAHDAADQYYGSAPYCYMQQQQHYGCYDGGAMVAGGDFFVPEEQLVADPSFMVDLNLEFEDQHGGDAGGAGSSAAAAAAATKMTPACKRKVEDHKDESCTDNVARKKARSTAATVVQKKGNKNAQSKKAQKGACSRSSNQKESNGGGDGGNVQSSSTNYLSDDDSLSLEMTSCSNVSSASKKSSLSSPATGHGGAKARAGRGAATDPQSLYARKRRERINERLKILQNLIPNGTKVDISTMLEEAVHYVKFLQLQIKLLSSDDMWMFAPIAYNGVNVGLDLKISPPQQQ

SEQ ID NO: 63 - DNA, 975 bp, Oryza sativa
atggaggactcggaggcgatggcgcagctgctcggcgtgcagtacttcggcaatgaccaggagcagcagcagccggcggcggcggcgccgccggcgatgtactggccggcgcacgacgcggccgaccagtactacggctcggcgccatactgctacatgcagcagcagcagcattacgggtgctacgacggcggcgcgatggtggccggcggcgacttcttcgtgccggaggagcagctggtggccgacccgagcttcatggtggacctgaacctcgagttcgaggaccagcacggcggcgatgctggcggcgctgggagcagcgccgccgccgccgccgccgccaccaagatgacaccggcgtgcaagaggaaggttgaggatcacaaggatgagagctgcacggacaacgtcgcgaggaagaaggcgcgctccacggcagcaacagtggtgcagaagaagggtaataagaacgcgcagtcaaagaaggcgcagaagggcgcgtgcagccggagcagcaaccagaaggagagcaatggcggcggcgacggcggcaatgtgcagagctcgagcaccaactacctctctgatgacgactcgctgtcgctggagatgacttcgtgcagcaacgtgagctcggcgtccaagaagtcgtcgttgtcatcgccggcgaccgggcacggcggcgcgaaggcgagggccgggcgcggggcggcgaccgatccgcaaagcctctatgccaggaagaggagagaaaggatcaatgaacggctaaagatactgcagaatcttatcccaaatggaaccaaggtggacatcagcacgatgcttgaagaagcagttcactacgtcaagttcttgcagctccaaatcaagcttctgagctcggatgatatgtggatgttcgcgccgatcgcgtacaacggggtcaacgtcgggctcgacctcaagatctctccaccgcagcagcaatga

SEQ ID NO: 64 - protein, 310 aa, Oryza sativa
MESGGVIAEAGWSSLDMSSQAEESEMMAQLLGTCFPSNGEDDHHQELPWSVDTPSAYYLHCNGGSSSAYSSTTSSNSASGSFTLIAPRSEYEGYYVSDSNEAALGISIQEQGAAQFMDAILNRNGDPGFDDLADSSVNLLDSIGASNKRKIQEQGRLDDQTKSRKSAKKAGSKRGKKAAQCEGEDGSIAVTNRQSLSCCTSENDSIGSQESPVAAKSNGKAQSGHRSATDPQSLYARKRRERINERLKILQNLVPNGTKVDISTMLEEAMHYVKFLQLQIKLLSSDEMWMYAPIAYNGMNIGIDLNLSQH

SEQ ID NO: 65 - DNA, 933 bp, Oryza sativa
atggagtccggaggggtgatcgcggaggcggggtggagctcgctcgacatgtcgtcgcaggccgaggagtcggagatgatggcgcagctgcttggaacctgcttcccctccaatggcgaggatgatcatcaccaagagcttccttggtcggttgacacccccagtgcctactacctccattgcaatggaggtagctcaagtgcatacagctctaccactagcagcaacagtgctagtggtagcttcactctcattgcaccaagatctgagtatgaggggtactatgtgagtgactctaatgaggcggccctcgggatcagcatccaggagcaaggtgcagctcagttcatggatgccattctcaaccggaacggcgatccgggcttcgatgatctcgctgactcgagcgttaatctgctggattccatcggcgcttctaacaagagaaagattcaggagcaaggcaggctagatgaccaaacgaaaagtaggaaatctgcgaagaaggctggctcgaagcggggaaagaaggcggcgcaatgtgaaggtgaagatggcagcattgctgtcaccaacaggcaaagcttgagctgctgcacctctgaaaatgattcgattggttctcaagaatctcctgttgctgctaagtcgaatggcaaggctcaatctggccatcggtcagcaaccgatccccagagcctctatgcaaggaaaagaagagagaggatcaatgagaggctcaagattctgcagaaccttgtaccaaatggaaccaaagtagatatcagcactatgcttgaagaggcaatgcattacgtgaagttcttgcagcttcaaatcaagctcctcagctctgatgaaatgtggatgtacgcaccgattgcttacaacgggatgaacatcgggatcgatttgaacctctctcagcattga

SEQ ID NO: 66 - protein, 246 aa, Oryza sativa
MDARCANIWSSADARSEESEMIDQLKSMFWSSTDAEINFYSPDSSVNSCVTTSTMPSSLFLPLMDDEGFGTVQLMHQVITGNKRMFPMDEHFEQQQKKPKKKTRTSRSVSSSSTITDYETSSELVNPSCSSGSSVGEDSIAATDGSVVLKQSDNSRGHKQCSKDTQSLYAKRRRERINERLRILQQLVPNGTKVDISTMLEEAVQYVKFLQLQIKLLSSDDTWMFAPLAYNGMNMDLGHTLAENQE

SEQ ID NO: 67 - DNA, 741 bp, Oryza sativa
atggatgcaaggtgtgcaaacatctggagctctgctgatgcaaggagtgaggaatctgagatgattgatcaactaaagtccatgttctggagcagcactgatgctgaaatcaacttttattctcctgacagtagtgtaaattcttgtgtcacaactagcacaatgcctagcagcttgtttcttcctctgatggatgatgagggatttggcacagtgcaattgatgcatcaggtcatcactgggaacaagaggatgttccccatggatgagcactttgagcagcagcagaagaagccgaagaagaaaacccgaacttctcgctcggtatcaagtagttcaaccattactgactatgagactagctctgaacttgtcaatcctagctgttcctccgggagcagcgtcggagaggattcaattgctgcaactgatggatctgtagtgctgaaacaaagtgacaattcaagaggccataagcagtgctccaaggatacacaaagcctctatgctaagaggagaagggaaaggattaatgagagactgagaatacttcagcagcttgttcccaatggcactaaagttgacatcagcacaatgctggaggaagcagttcagtatgtcaagtttttgcagttgcaaataaagctattgagctctgacgacacatggatgtttgcgcccctagcctataatggcatgaacatggatctcggtcatactcttgctgaaaaccaagaatga

SEQ ID NO: 68 - protein, 198 aa, Oryza sativa
MECSSFEAICNESEMIAHLQSLFWSSSDADPCFGSSSFSLISSEGYDTMTTEFVNSSTNVCFDYQDDSFVSAEETTIGNKRKVQMDTENELMTNRSKEVRTKMSVSKACKHSVSAESSQSYYAKNRRQRINERLRILQELIPNGTKVDISTMLEEAIQYVKFLHLQIKLLSSDEMWMYAPLAFDSGNNRLYQNSLSQE

SEQ ID NO: 69 - DNA, 597 bp, Oryza sativa
atggaatgcagctcctttgaagcaatctgcaatgagtcggagatgattgcgcatttgcagtcattgttctggagcagcagcgatgctgatccttgttttggtagctcatcattttctctcatcagtagtgagggctacgacacaatgaccacagagtttgtgaatagcagcacaaatgtatgttttgattaccaagatgatagcttcgtttcagcagaggagactaccattggtaacaagagaaaagttcagatggatactgagaatgagctgatgacgaaccgcagcaaggaagttcgcaccaagatgtcggtgtcaaaagcatgcaaacattctgtttctgcagagagctcacagtcttattatgcaaagaacaggagacagaggatcaatgagagattgagaatactgcaagaactgatccctaatggaacaaaagttgacatcagcacaatgttggaggaagcaattcagtatgtcaagtttctacacctgcaaatcaagctcttgagctctgatgaaatgtggatgtatgcgccccttgcttttgacagtggtaacaacaggctctatcagaactctctgtcacaagagtag

SEQ ID NO: 70 - protein, 304 aa, Oryza sativa
MEGGGLIADMSWTVFDLPSHSDESEMMAQLFSAFPIHGEEEGHEQLPWFDQSSNPCYYSCNASSTAYSNSNASSIPAPSEYEGYCFSDSNEALGVSSSIAPHDLSMVQVQGATEFLNVIPNHSLDSFGNGELGHEDLDSVSGTNKRKQSAEGEFDGQTRGSKCARKAEPKRAKKAKQTVEKDASVAIPNGSCSISDNDSSSSQEVADAGATSKGKSRAGRGAATDPQSLYARKRRERINERLKTLQNLVPNGTKVDISTMLEEAVHYVKFLQLQIKLLSSDEMWMYAPIAYNGMNIGLDLNIDT

SEQ ID NO: 71 - DNA, 915 bp, Oryza sativa
atggagggtggaggactgatcgccgatatgagctggaccgtcttcgacttgccatcgcacagcgatgagtcggagatgatggcgcagctcttcagtgcattccccatccatggtgaggaggaaggccatgagcagctcccatggtttgatcaatcttccaatccatgctactatagctgcaatgctagcagcactgcatacagcaacagcaatgctagtagcattcctgctccatctgagtatgaaggatactgcttcagtgactcaaatgaggccctgggtgtcagctccagcattgcaccacatgacctgagcatggtccaggtgcaaggtgcaactgagtttctgaatgtgatcccaaaccattcccttgattcattcggtaatggcgagctgggccacgaggatcttgattcggttagtgggactaacaagagaaaacagtcggcagaaggagaatttgatggccaaacaagaggttcaaaatgcgcgagaaaggctgaaccgaagcgagcgaagaaggccaagcaaactgtggagaaggatgcaagtgttgccatcccaaatgggagctgttccatttctgacaatgattccagttcatcccaggaggttgcagatgctggtgctacttcgaaaggcaaatcccgggctggccgcggagcagccactgatccccagagcctctatgcaaggaaaaggagagagaggatcaatgagaggctcaagacacttcagaaccttgtgcccaatggcaccaaagttgatatcagcaccatgcttgaggaggcagtccactatgtgaagttcctgcagcttcagatcaagctcctcagctccgatgaaatgtggatgtatgcgccaattgcgtacaacgggatgaacattgggctcgatctgaacattgatacatga

SEQ ID NO: 72 - protein, 279 aa, Oryza sativa
MAQFLGAHGDHCFTYEQMDESMEAMAAMFLPGLDTDSNSSSGCLNYDVPPQCWPQHGHSSSVTSFPDPAHSYGSFEFPVMDPFPIADLDAHCAIPYLTEDLISPPHGNHPSARVEEATKVVTPVATKRKSSAAMTASKKSKKAGKKDPIGSDEGGNTYIDTQSSSSCTSEEGNLEGNAKPSSKKMGTRANRGAATDPQSLYARKRRERINERLRILQNLVPNGTKVDISTMLEEAVQYVKFLQLQIKLLSSDDTWMYAPIAYNGVNISNIDLNISSLQK

SEQ ID NO: 73 - DNA, 840 bp, Oryza sativa
atggcgcagtttcttggagctcatggtgatcactgcttcacctacgagcaaatggatgagtccatggaggcaatggcagcgatgttcttgcctggccttgacaccgactccaattcttcttctggttgtctcaactacgatgtgcctccacaatgctggcctcagcatggccatagctctagcgtcaccagcttccctgatccagctcatagctatggaagctttgagttcccggtcatggatccgttcccgatcgccgatctcgacgcgcattgcgccatcccctaccttactgaggatctgatcagccctccacatggcaaccatccatcagcaagagtggaagaagctacaaaggttgttacaccagtggctaccaagaggaagtctagtgctgccatgacggcatcaaagaagagcaagaaggctggcaaaaaagatcctattggcagcgacgaaggcggcaacacctacattgatacgcaaagttctagcagttgcacctcagaggaaggaaacctggagggcaacgcgaagccgagctcgaagaagatgggtactagggccaaccgtggggcggcaaccgatccccagagtctctatgcaaggaagaggagagagaggatcaatgaaagattgaggatcctgcagaacttggttcccaatggaacaaaggttgacatcagtacaatgctggaggaagcagtgcagtatgtcaaatttttgcaacttcagattaagttgctaagctctgatgacacgtggatgtatgcaccaatcgcttacaatggagtcaacatcagcaatattgatctgaacatctcttctctgcaaaaataa

SEQ ID NO: 74 - protein, 334 aa, Populus trichocarpa
MALAKDRMGSVQTCPYNGNVMGDFSSMGSYGFDEYQKVAFYEEGNSTFEKTSGLMIKNLAMTSSPSSLGSPSSAISGELVFQATDHQAEEAHSLISFKGIGFDNIMHNNGSLLSFEQSSRVSQTSSQKDDYSAWEGNLSYNYQWNEMNPKCNTSPRLMEDFNCFQRAGNFISMTGKENHGDWLYAESTIVADSIQDSATPDASSFHKRPNMGESMQALKKQCNNATKKPKPKSAAGPAKDLQSIAAKNRRERISERLKVLQDLVPNGSKVDLVTMLEKAISYVKFLQLQVKVLATDELWPVQGGKAPDISQVKEAIDALLSSQTKDGNSSSSPK

SEQ ID NO: 75 - DNA, 1005 bp, Populus trichocarpa
atggcacttgccaaggaccgtatgggatcggttcaaacttgcccctataatggaaatgtgatgggggatttttcctccatggggtcttacggatttgatgaatatcagaaggtagcattttatgaagagggaaatagcacctttgagaaaaccagtgggcttatgatcaagaatttagctatgacctcttctccttcttctcttggcagtccgagcagcgcgatttctggtgaattagtgtttcaggctactgaccatcaagctgaggaagctcattctttgatcagcttcaaaggtatcggattcgataacatcatgcataataatggatctttgcttagctttgagcaaagtagtagggtttctcaaactagtagccagaaagatgactactcagcctgggagggtaatttgagttacaactaccagtggaacgaaatgaatccaaaatgtaacacaagtcctcggttgatggaagattttaattgctttcaaagagctggcaacttcatttccatgactggaaaggaaaatcatggtgattggttatacgctgaatccacaattgttgctgatagcattcaggattctgcaacaccagatgccagcagcttccataagcgtcctaatatgggagagagtatgcaggctctaaagaagcaatgcaacaatgcaacaaaaaagccaaaaccgaagtccgcagcaggtccagctaaggatctacagagtattgctgccaagaatcgacgagagaggattagcgagaggcttaaggtattgcaggatttagtccctaatggctcaaaggttgatttggttactatgctagagaaagccattagttatgttaagtttcttcaattgcaagtaaaggtgttagccactgatgaattatggccagttcaaggtggtaaagctcctgatatttctcaagtaaaggaagccatcgatgccctactctcatctcagactaaagacggaaactcaagctcaagcccaaagtaa

SEQ ID NO: 76 - protein, 330 aa, Populus trichocarpa
MALAKDRMDSVQTCALYGNVMGDLSSLGPNYRFDEEGDRNFEKNSALMIKNLAMSPSPPSLGSPSSANSGELVFQATDNQVEEAHSLINFKGTGFDSIMHANGSLISFEQSNRVSQTSSHKDDYSAWEGNLSCNYQWNQINPKCNANPRLMEDLNCYQSASNFNSITNSAEKENHGDWLYTHESTIVTDSIPDSATPDASSFHKRPNMGESMQALKKQRDSATKKPKPKSAGPAKDPQSIAAKNRRERISERLKMLQDLVPNGSKVDLVTMLEKAISYVKFLQLQVKVLATDEFWPVQGGKAPDISQVKGAIDATLSSQTKDRNSNSSSK

SEQ ID NO: 77 - DNA, 993 bp, Populus trichocarpa
atggcacttgccaaggaccgtatggattcggttcaaacttgcgccctttatggaaatgtgatgggggatctttcctccttggggcctaattatagatttgatgaagagggagataggaactttgagaaaaatagtgcgcttatgatcaagaatttagctatgagcccttctcctccttctcttggcagtccaagcagtgcaaattctggtgaactagtgtttcaggctactgacaatcaagttgaggaagctcattctttgatcaacttcaaaggtaccggatttgatagtatcatgcatgctaatggatctttgattagctttgagcaaagtaatagggtttctcaaactagtagtcacaaagatgactactctgcttgggagggtaatttgagttgcaattaccagtggaaccaaatcaatccaaaatgtaacgcaaatcctcggttgatggaagatcttaattgctatcaaagtgcaagcaacttcaactccataaccaacagtgctgaaaaggaaaaccatggtgattggttatacactcatgaatccacaattgttactgatagcattcccgattctgcaacaccagatgccagcagcttccataagcgtcccaatatgggagagagtatgcaggctctaaagaagcaacgcgacagcgccacaaaaaagccgaaacccaagtctgctggtccagctaaggatccacaaagtattgctgccaagaatcgacgagagcggattagcgagcgccttaagatgttgcaggatttagtccctaacggctccaaggttgatttggttactatgctagagaaagccattagttatgttaagtttcttcaattgcaagtaaaggtgttggccactgatgaattctggccagttcaaggtggtaaagctcctgatatttctcaagtaaagggagccattgatgccacactctcatctcagactaaagacagaaattcaaactcaagctcaaagtga

SEQ ID NO: 78 - protein, 354 aa, Populus trichocarpa
MAEGEWSSLGGMYTSEEADFMAQLLGNCPNQVDSSSNFGVPSSFWPNHEPTTDMEGANECLFYSLDFANINLHHFSQGSSSYSGGSGILFPNTSQDSYYMSDSHPILANNNSSMSMDFCMGDSYLVEGDDCSNQEMSNSNEEPGGNQTVAALPENDFRAKREPEMPASELPLEDKSSNPPQISKKRSRNSGDAQKNKRNASSKKSQKVASTSNNDEGSNAGLNGPASSGCCSEDESNASHELNRGASSSLSSKGTATLNSSGKTRASRGAATDPQSLYARKRRERINERLRILQTLVPNGTKVDISTMLEEAVQYVKFLQLQIKLLSSEDLWMYAPIAYNGMDIGLDHLKVTAP

SEQ ID NO: 79 - DNA, 1065 bp, Populus trichocarpa
atggcagagggagagtggagttctcttggtggaatgtacactagtgaggaggctgatttcatggcacagttgcttggtaactgtcctaatcaggttgattcaagttcaaactttggagttccatctagtttctggcctaaccacgaaccaacaacggacatggaaggggctaatgaatgtttattttattctttggattttgctaatattaatttgcaccatttttcacaagggagtagtagttatagtggtggcagtggcattctttttcccaacacaagccaagatagctactacatgagtgattctcatccaattttggctaacaataatagctcaatgtcaatggatttttgcatgggagactcatatctcgttgaaggcgatgactgctcaaaccaagaaatgagcaatagcaatgaggagcctggtggaaaccagactgtagctgctcttcctgaaaacgattttcgggccaagagagaaccagagatgccagcttctgaactacccctggaagacaaaagcagcaacccacctcagatttctaagaaaagatcacgaaattcaggagatgctcaaaagaacaagaggaatgcaagttcaaagaagagccagaaggttgcctcgactagcaacaatgatgaaggaagtaatgctggccttaatgggcctgcctcaagcggttgctgctcagaggatgaatccaatgcctctcatgagctcaatagaggagcgagttcaagtttgagctcgaaagggactgcaactctcaactcaagtggcaaaacaagagccagcaggggggcagccactgatccccagagtctctatgcaaggaaaagaagagaaagaataaatgagaggctgagaattctacaaacccttgtccccaacggaacaaaggttgacattagcacaatgcttgaagaagctgtccagtatgtgaagtttttgcaactccaaattaagctgctaagctctgaggacttgtggatgtatgcgcctatcgcttacaacgggatggacatcggtcttgatcatctgaaggttaccgcaccatga

SEQ ID NO: 80 - protein, 317 aa, Populus trichocarpa
MEPIGATAEGEWSSLSGMYTSEEADFMEQLLVNCPPNQVDSSSSFGVPSSFWPNHESTMNMEGANECLLYSLDIADTNLYHFSQVSSGYSGELSNGNVEESGGNQTVAALPEPESNLQPKRESKMPASELPLEDKSRKPPENSKKRSRRTGDAQKNKRNVRSKKSQKVASTGNNDEESNGGLNGPVSSGCCSEDESNASQELNGGASSSLSSKGTTTLNSSGKTRASKGAATDPQSLYARKRRERINERLRILQNLVPNGTKVDISTMLEEAVQYVKFLQLQIKLLSSEDLWMYAPIAYNGMDIGLDHLKLTTPRRL

SEQ ID NO: 81 - DNA, 954 bp, Populus trichocarpa
atggagcctattggagccactgcggagggagagtggagttctcttagtggaatgtacacaagtgaggaggctgatttcatggaacagttgcttgtcaactgtcctcctaatcaggttgattcaagttcaagctttggagttccatctagtttttggcctaaccatgaatcaacaatgaacatggaaggggccaatgaatgtttattgtattctttggatattgctgatactaatctgtaccatttttcacaagtgagcagtggttatagtggtgaattgagcaatggaaatgtggaagagtctggtggaaaccagactgtagctgctcttcctgaacctgaaagcaatttgcaacccaagagagaatcaaagatgccagcatctgaactacccctggaagataaaagcagaaagccacctgagaattccaagaaaagatcacgacgtacgggagatgcccaaaagaacaagaggaatgtaaggtcaaagaagagccagaaggttgcctcgactggcaacaatgatgaagaaagcaatggtggccttaatggtcctgtctcaagcggttgctgctcagaggatgaatccaatgcctcccaggagctcaatggaggagcgagttcaagtttgagctcaaaagggacaacaactctcaactcaagtggcaaaacaagagccagtaagggggcagccactgatccccagagcctctatgcaaggaaaagaagagaaagaataaatgagaggctgagaattctacaaaaccttgtccccaatggaacaaaggttgacattagcacaatgcttgaagaggctgtccagtatgtgaagtttttgcaactccaaattaagctgctaagctctgaagacctgtggatgtatgctcctatcgcgtacaatggtatggacatcggtcttgatcatctgaagcttaccacaccaagacgattgtag

SEQ ID NO: 82 - protein, 362 aa, Populus trichocarpa
MNTQAMEAFRDGELWNFSRMFSMEEPDCTPELLGQCSFLQDTDEGLHFTIPSAFFPAPESDASMAEDESLFYSWHTPNPNLHFDSQESSNNSNSSSSVFLPYSSHESYFFNDSNPIQATNNNSMSMDIMDEENIGLFMPLFPEIAMAETACMNGDMSGDKTGDLDDNLKPAANDVLAKGLQLKRKLDVPEPIANTLDDMKKKARVTRNVQKTRKVGQSKKNQKNAPDISHDEEESNAGPDGQSSSSCSSEEDNASQDSDSKVSGVLNSNGKTRATRGAATDPQSLYARKRRERINERLKILQNLVPNGTKVDISTMLEEAVHYVNFLQLQIKLLSSDDLWMYAPLAYNGIDIGLNQKLSMFL

SEQ ID NO: 83 - DNA, 1089 bp, Populus trichocarpa
atgaatacgcaggctatggaagcctttcgtgatggagaattatggaacttcagcagaatgttctccatggaagagcctgattgcaccccagaattacttggtcagtgctcttttcttcaggatactgatgaaggattgcattttacaatcccatcagctttcttccctgctcctgaatccgacgcgagcatggctgaggacgagagtttgttttattcttggcatactcccaaccccaatttgcattttgattctcaagaaagtagtaataacagtaattctagcagtagtgtatttcttccctattccagccatgaatcctacttcttcaatgattctaatcccattcaagctacgaacaataactctatgtccatggatattatggatgaggaaaatattggcttgtttatgccactttttcctgaaattgcaatggcagaaactgcctgtatgaatggagatatgagcggtgacaaaacaggagatttagatgataatctgaagccagcagctaatgatgttctggccaagggattgcagctcaaaaggaagcttgatgttccagaaccaatagccaacacattggacgacatgaagaaaaaagcccgggttacaagaaatgtgcaaaagactaggaaggttggacagtcaaaaaaaaatcagaagaacgcaccagatattagccatgatgaagaagagagtaatgctggaccagacggacaaagttccagcagttgtagttcagaagaggacaatgcctctcaggattctgattccaaggtttctggagttctcaattccaatggaaaaacaagagctactaggggagctgccacagacccccagagcctttatgcaaggaaaagaagggagaggataaacgagagactgaaaatcttgcagaatcttgtccctaacggaaccaaggttgatatcagcacgatgctagaagaggcagtccattacgtaaactttttgcagcttcaaatcaagcttttgagctcggatgatctatggatgtatgcacctctggcttacaatggaatagatattggactcaaccagaagctctctatgtttctatga

SEQ ID NO: 84 - protein, 302 aa, Musa acuminata
MAQESTWSSFDATMLAEEESRMIAQLLSNYQCFGEQDRDVGCCELPPSSCCSSHAADSCYCWSANENSNPGLCYWSQSGDESDGAHAIGTVPVFTNHCLVGDQVAVNQTLSIHEPTAAHAEMPKRKIESHASEDDFRRQSSKKKLQAPTNALKSVKKARPGRNQKSIVCGDEEENNARSSGRSCCSYSSEEDSQAFQADLNAKTRSNRWPATDPQSLYAKQRRERINARLRTLQNLVPNGTKVDISTMLEEAVRYVKFLQLQIKLLSSDELWMYAPVVHSGMIDGQVNSEIFVSANTRNEWF

SEQ ID NO: 85 - DNA, 909 bp, Musa acuminata
atggctcaggagtcaacttggagctcgtttgatgctacaatgcttgctgaggaggagtcccgaatgatcgcacaattgctcagcaactaccagtgttttggcgagcaagatcgagatgttggatgctgtgaactcccgccatcgtcttgttgttcttctcatgcagctgattcatgttactgttggtcagcaaatgagaacagtaacccgggtttgtgctactggtctcagagtggagatgaatccgatggagcacatgcaatcggcactgtgccggtcttcacgaaccattgcttggtgggagatcaagtcgctgtgaatcaaactttgagcattcacgaacctactgctgctcatgcagagatgccaaagcgcaagatagagtctcatgcttctgaagatgatttccgtcgtcaaagttctaagaaaaagcttcaggctccgacgaatgctctgaagagcgtgaagaaggcacgacctgggaggaaccagaagagcattgtgtgtggtgatgaggaagagaacaatgccaggagcagtggccggagttgctgcagctacagctctgaggaagactcacaagctttccaggctgatcttaatgcaaaaacacgatcgaatcgatggccagccacagatcctcaaagcctctatgcaaagcaaagaagggaaagaatcaatgctagattgaggacattgcagaacctggtgcctaatggaactaaagttgacattagcacaatgctcgaagaagctgttcgttacgtcaagttcttgcagctgcagataaagcttttgagctcggatgagctgtggatgtacgctcctgttgtccacagtgggatgattgatggccaagtcaactcagagatatttgtgtctgcaaatactcgtaatgagtggttctga

SEQ ID NO: 86 - protein, 366 aa, Medicago truncatula
MEPIGTFPEGEWDFFRKMFASEDHEYYSQQFLDQNSLLLGENDGLNNGTQSTFCTAEIGENERMFYSFDHAHIQNSNYIPQTQENSYNSNSSASDDTNYYFSYPNHVLENNINNCISNDFRMDENLFASSVPSLNEIVMEENVRMNEDSASDDHIVEKNGYNTQIMEPFDLHTKHEMQMKLKRKLDVIEVEVPVEEKINNNPKKKPRVSNDGQGCMKNARSKKNHKVIASHEEEMTEEINRGSNGNSSSSNISEDDNASQENSGGTTLNSNGKTRASRGSATDPQSLYARKRRERINERLRVLQNLVPNGTKVDISTMLEEAVNYVKFLQTQIKLLSSDDMWMYAPLAYNGLDLGLNLNLNSSLPL

SEQ ID NO: 87 - DNA, 1101 bp, Medicago truncatula
atggaacctataggtactttccctgaaggagaatgggatttctttcgcaaaatgtttgcaagtgaagatcatgaatattactcacaacaatttcttgatcaaaattcacttcttctaggggaaaatgatgggttgaacaatggaacacagtccacattttgcactgctgaaattggtgaaaatgagcgtatgttttattcttttgatcatgctcatatccaaaactctaactatattcctcaaactcaagagaatagttacaatagcaattctagtgctagtgatgatacaaattactattttagttatcctaatcatgtactagaaaataatattaataattgtatatccaatgattttcgcatggatgagaatttgtttgcttcttctgttccatcccttaatgagattgtaatggaagagaatgtgagaatgaatgaagattctgcaagtgatgatcatattgtggagaaaaatggttacaatactcaaataatggaaccttttgatcttcacaccaagcatgagatgcaaatgaagctcaaaaggaaacttgatgtgatagaagtggaggttcccgttgaagaaaaaattaacaacaatccgaagaaaaaacctcgtgtttcgaatgatggccaaggatgcatgaaaaatgcaaggtcaaagaagaaccacaaagttattgctagccatgaagaggagatgacagaagagattaatagaggatcaaatggaaatagttctagtagtaacatttctgaggatgataatgcttctcaagaaaatagtggaggaactactctcaactcaaatgggaagacaagagctagtagaggatctgcaacagatccccaaagtctatatgcaaggaaaagaagagagagaataaatgaacgactaagagtcttacaaaatcttgtaccaaacggaacaaaggttgatatcagtacaatgcttgaagaggcagtcaattatgtgaaatttttacagactcaaatcaagcttttgagctctgatgatatgtggatgtatgcaccacttgcttacaatggacttgaccttggactcaatctcaacctcaacagctctctaccactatga

SEQ ID NO: 88 - protein, 258 aa, Glycine max
  misc_feature (1)..(1): Xaa is unknown
XFLCFSQGSSSSTDNSGNNIFSITSSGAYSCDPEANFDSVSMVLCLGDAKFSPHSFQCDDNSNQQINENTDEESSLDPWKLAIADNNLQAKREYEMMVSEPVEVDRSRNLENLAKRLKSSIEVSKTLRSAKSGKNSKSASVSNDEDDRSLSLQAQRNSCFSQSDSNAYLEPNGGASKDPAPPNLHRKSRATTGAATDPQSLYARKRRERINERLRILQNLVPNGTKVDISTMLEEAVQYVKFLQLQIKLLSSDDLWMY

SEQ ID NO: 89 - DNA, 773 bp, Glycine max
attttttgtgtttctcacaagggagtagctccagtactgataatagtggtaataatatcttttccattacaagtagtggagcctactcctgtgatccagaagcaaactttgattctgtgtccatggttttgtgccttggagatgccaaatttagtccccatagttttcaatgtgatgacaactcaaaccaacagataaatgaaaacactgatgaagagtcaagtctagacccatggaagttggctatagctgacaataatttgcaggctaagagggagtatgaaatgatggtttctgaacctgtagaagtggatagaagcagaaacctggagaacctagcaaaaagactaaagagttcaatagaggtttcaaaaacattgaggagtgctaaatcagggaaaaattcaaaatctgcttcagtgagcaacgatgaagatgatagaagcttgagcctccaagcccaaaggaatagctgtttttcacagagtgactctaatgcttatctggagccaaatggaggggcatcaaaagatcctgcacctcccaatttgcatagaaaatcaagagcaactaccggtgctgccactgatccacagagcctctatgcaagaaagagaagagaaagaataaatgaaaggttgagaatactgcaaaatcttgttcccaacggaactaaggtggatatcagcaccatgcttgaggaagctgtccaatacgtgaagtttttacagctccaaattaagcttctgagctctgacgatctgtggatgtat

SEQ ID NO: 90 - protein, 176 aa, Glycine max
  misc_feature (1)..(1): Xaa is unknown
XNLENLPKRLKSSIEVPKTSRNAKSRKNSKSASTSNDEDDRSLSLQVQRNNSCFSQSDSNAYLEPNGGASKDPAPPNLDRKSRATTSAAADPQSLYARKRRERINERLRILQNLVPNGTKVDISTMLEEAVQYVKFLQLQIKLLSSEDLWMYAPIVYNGINIGLDLGISPTKGRSM

SEQ ID NO: 91 - DNA, 571 bp, Glycine max
gaaacctggagaacctaccaaaaagactaaagagctcaatagaggtcccaaaaacatcgaggaatgctaaatcaaggaaaaattcaaaatctgcttcaactagcaacgatgaagatgatagaagcttgagcctccaagtccaaaggaataatagctgtttttcacagagtgactctaatgcttatcttgagccaaatggaggggcatcaaaagatcctgcacctcctaatttggatagaaaatcaagagcaactaccagtgccgccgctgatccacagagcctctatgcaagaaagagaagagaaagaataaatgaaaggctgagaatactgcaaaatcttgtccccaacggaactaaggtggatatcagcaccatgcttgaagaagctgtccaatacgttaagtttttacagctccaaattaagcttctgagctctgaagatttgtggatgtatgctccaattgtttacaatggaataaacattggactagacctcggtatttctccaaccaaaggaagatcaatgtgatagcatagcaattaaagaggatataatatttcattaactta

SEQ ID NO: 92 - protein, 108 aa, Lactuca saligna
  misc_feature (1)..(1): Xaa is unknown
XRSKEAEILSSNGKRKASRGSATDPQSVYARKRRERINERLRILQNLVPNGTKVDISTMLEEAVEYVKFLQLQIKLLSSDDMWMYAPIAYDGMDIGLHSTTIPSSSTR

SEQ ID NO: 93 - DNA, 419 bp, Lactuca saligna
tgagatcaaaagaggctgaaattctgagctcaaatggcaagagaaaagcaagtagggggtcagcaactgatccacaaagtgtctatgcacggaaaagaagagaaagaattaacgaacgtttaagaatattacaaaatcttgttcctaatggtacaaaggttgatataagcacaatgcttgaagaggctgttgagtacgtgaagtttttgcagcttcaaatcaagctcttgagctccgatgatatgtggatgtatgctccgattgcatacgatggaatggacattgggcttcattcaacaaccatcccatcatcgtcaacaagataatgcaaagttgggctatccatattgtcacatttttgttgaataaaaggcaatcgataacaaaattcaaagtttataaagagtacacatttatgc

SEQ ID NO: 94 - protein, 274 aa, Triticum aestivum
MASKRATTRELRAMYDDEPSSMSLELFGYHGVVVDGDDENDDTATALPQLSFVDNFKGGCGSAADYYSWAYNASGGTPGASSSSTSSVLSFEHAGGAGHQLAYNSGTGDDDCALWMDSMADHQHGAARFGFMNPGSADVVPEIQESSIKQPAKSAQKRSSSGGEAQAAAKKQCGGGRKSKAKVVPTKDPQSAVAKVRRERISERLKVLQDLVPNGTKVDMVTMLEKAITYVKFLQLQVKVLATDEFWPVQGGKAPELSQVKTALDAILSSQQQP

SEQ ID NO: 95 - DNA, 825 bp, Triticum aestivum
atggcgagcaagcgggccaccacgcgggagctccgggcgatgtacgacgacgagccctcctccatgtccctcgagctcttcggctaccatggcgtggtcgtcgacggtgacgatgaaaacgacgacactgccaccgccctgccccagctctccttcgtcgacaacttcaaaggtgggtgcgggtcggcggcggactactacagctgggcgtacaacgcctccggcgggacgccgggcgcctcctccagctccacctcgtcggtgctcagctttgagcatgccggcggtgccggtcatcagctggcttataattccggcacaggcgacgatgactgcgcgctctggatggacagcatggccgatcatcagcacggcgcggccaggtttgggttcatgaacccagggtcggccgatgtcgtcccagaaatccaggagagcagcatcaagcagccggccaagtctgcgcagaagcgctcgagctcgggtggtgaggcgcaagcagcggcgaagaagcagtgtggaggaggcaggaagagcaaggccaaagttgtccctaccaaggatcctcagagcgctgttgcaaaggtccgaagagagcgcatcagtgagaggctcaaagttctgcaggatcttgtacccaacggcacgaaggtggacatggtcaccatgctcgagaaggcaatcacctatgtcaagttcctgcagctgcaagtcaaggtgttggcgaccgacgagttctggccggtgcaaggagggaaggcgccggagctctcccaagtgaagaccgcgctggacgccatcctttcttcccagcagcaaccctag

SEQ ID NO: 96 - protein, 18 aa, Carthamus tinctorius
DSQIIHPMPCDELHKSLI

SEQ ID NO: 97 - DNA, 769 bp, Carthamus tinctorius
gattcacagataatccaccctatgccgtgtgatgaactccacaaatccttaatttaattgtaccacatcaggcgacgttatccatattgggtgttcactgatggtgaaagcacatctttcgcgcgacctctactcaatgactcaagaattagaggtgaactattgcttacactatctactactaaacattgtaaagtgactgccagttctatgagacgttcgtatagcatgatgcatgatcatgagaaaagctaaaagatacagcgcagaaagagccagaagctcgtttctaaaggcaacgaaagtgaagctgaccatgatgcagtttttgggcaaataatgaaaatgtgtggatctgacaatgactcgaattggcctcgggagtcgagcacaagtccaagaccaaaagaggctgcaaatctgaactcaaatgggaagacaaaagcaaatagggggtcagcaacggatccacaaagtgtctacgcacggaagagaagagaacgaattaatgaacggttaagaatactacagagtctggttcctaatggtacaaaggttgatataagcacaatgcttgaagatgctgtccagtatgtgaaatttttgcagctccaaatcaagccgttgagctctgatgatctgtggatgtatgcccccatcgcgtacaacgggatggagacggggcttgattctacgatcccctcgccaaggtgaagactatccaaagttgccgcatcttttttcttgaaaaaagggaagcctggggcaa

SEQ ID NO: 98 - protein, 309 aa, Brachypodium distachyon
MALVREPMVLYDGGFDASEASAFDSIGCFGHGHGHDALLGGVDAAALFGGYAHDEPAGASASAYVKDGSHWAGVGASVLAFDRAARGHGAQAMATAAAQEEEECDAWIDAMDEDNGEAAPAPSIGFDPATGCFSLTQRPGAGARRPFGLLFPSASGGAPSPDSAAPAPASRGSQKRPSAGIARAQDAEPRASKKQCGASRKTTAKAKSPAPAITSPKDPQSLAAKNRREKISERLRTLQEMVPNGTKVDMVTMLEKAISYVKFLQLQVKVLATDEFWPAQGGMAPEISQVKEALDAILSSQRGQFNCSS

SEQ ID NO: 99 - DNA, 930 bp, Brachypodium distachyon
atggcattagtgcgggagccgatggtactgtatgacggcggtttcgacgcctcggaggcgtcggcattcgactccatcggctgcttcggccacggccacggccacgacgcgctcctaggcggcgtcgacgcggccgcgctgttcgggggctacgcgcacgacgagccggccggcgccagcgccagcgcctacgtgaaggacggctcgcactgggccggcgtgggtgcgtccgtgctcgcgttcgaccgtgccgctcggggccacggcgcgcaggccatggcgaccgcggccgctcaggaggaggaagaatgcgacgcgtggatcgacgccatggacgaggacaatggcgaggcggcgccggcgccgtccatcggcttcgacccggccacgggctgcttcagcctcacgcagcggcccggcgccggcgcgcggcgcccgttcgggctcctgttcccgagcgcgtccggtggcgcgccctcgcccgacagcgccgcgccagcgccggcatcccgcggttcccagaagcggccatccgccgggattgcgcgcgcgcaggacgcggagccgcgggccagcaagaagcagtgcggcgcgagcaggaagacgacggccaaggcgaagtcgcctgcgcctgccatcacctcgcccaaggacccgcagagcctcgctgcaaagaaccggagggagaagatcagcgagcggctccggacgttgcaggagatggtgcccaacggcaccaaggtggacatggtcaccatgctcgagaaggccatcagctacgtcaagttcctgcagctgcaagtcaaggtgctcgcgacggacgagttctggccggcgcagggagggatggcgccggagatctcccaggtgaaggaggcgctcgacgccatcctgtcgtcgcagagggggcaattcaactgctccagctag

SEQ ID NO: 100 - protein, 272 aa, Brachypodium distachyon
MASRHATTREPHLRTMYDDEPSMSLELFGYHGVVVDGDDDGDTATDLPQLTFVDNFKGGCGSADYYGWAYSASGGASGACSSSSSSVLSFEQAGGAGHQLAYNAGTGDDDCALWMDGMADQHDTAKFGFMDPGMSDVSLEIQESSMKPPAKMAQKRACQGGETQAAAKKQCGGSKKSKAKAAPAKDPQSAVAKVRRERISERLKVLQDLVPNGTKVDMVTMLEKAITYVKFLQLQVKVLATDDFWPVQGGKAPELSQVKDALDAILSSQNQS

SEQ ID NO: 101 - DNA, 819 bp, Brachypodium distachyon
atggcaagcaggcacgccactacacgggagccacacctccggaccatgtacgacgacgagccatccatgtccctcgagctcttcggctaccatggcgtcgtcgtcgacggtgacgacgatggcgacaccgccaccgaccttccccagctcacctttgttgacaacttcaaaggcgggtgtgggtcagccgactactacggctgggcgtacagcgcctccggtggtgcgtcaggcgcctgctccagctccagctcgtcggtgctcagctttgagcaggcgggtggtgccggtcatcagctggcttataacgccggcacaggtgacgatgactgcgcgctctggatggacggcatggctgaccagcatgacacagccaagtttgggttcatggacccaggcatgtctgatgtcagcctagaaatccaggagagcagcatgaaaccgccggccaagatggcacagaagcgcgcttgccagggtggtgagacgcaagcagcggcgaagaagcagtgtggaggaagcaagaagagcaaggcaaaagctgcccctgccaaggatcctcaaagcgccgttgcaaaggtccgaagagagcgcatcagcgagaggctcaaagttctgcaggatctcgtgcccaatggcacaaaggttgacatggtcaccatgctcgaaaaggcaatcacctatgtcaagttcctgcagctgcaagtcaaggtattggcgactgatgacttctggccggtgcaaggagggaaagctccggagctctcccaagtgaaggacgctctggacgcgatcctgtcttcccagaatcaatcctag

SEQ ID NO: 102 - protein, 240 aa, Brachypodium distachyon
MALVGQATKLCYDGFAGDGVPPFMDAACLAFDHGYDYNNPHAWEFPTGAEPGNSSAFDVAWTGVSSTSPVLTFDAAEWMDATATDRLSSYSPSAATVPASYKRPRAHVQPQQEAEEQESITPNPKKQCGDGKVVIKSSAAATGTSPRKEPQSQAAKSRRERIGERLRALQELVPNGSKVDMVTMLDKAITYVKFMQLQLTVLETDAFWPAQGGAAPEISQVKAALDAIILSSSQKPRQWS

SEQ ID NO: 103 - DNA, 723 bp, Brachypodium distachyon
atggctctagtgggtcaggcaacgaagctctgctacgacggcttcgccggagacggtgtgccgccgttcatggacgcagcttgtctggcattcgaccacgggtatgattacaacaatccccacgcatgggaattccccaccggcgccgagccaggcaacagcagcgcgttcgacgttgcctggaccggcgtctcctccacttctccggtgctcacattcgacgccgccgagtggatggacgccacggccacggaccggctgagctcctacagcccgtctgcggccaccgtgccggcctcttacaagcggcctcgtgcgcacgtgcagccacagcaggaagcagaagaacaggaaagcattactcccaatcccaagaagcagtgcggcgatgggaaagtagttatcaagtcatcggcggcggctaccggcaccagtccacgcaaggaaccccaaagccaagctgccaagagccgtcgtgagcggatcggcgagcggctgagagcgctgcaggagctggtgcccaacggcagcaaggtggacatggtcaccatgctcgacaaggccatcacttatgtcaagttcatgcagctccagctcacggtgctcgagacagacgcgttctggcctgcgcagggtggcgcggcgccggagatctcccaggtgaaggcggcgctcgacgccatcatcctctcctcgtcgcagaagcctcgtcagtggagctag

SEQ ID NO: 104 - protein, 821 aa, Brachypodium distachyon
MEAGGLISEAGWTMFDFPSQGEESEIMSQLLGAFPSHLEEGHQDLPWYQASDPSYYDCNLNTSSESNASSLAVPSECMGYYLGDSSESLDLSSCIAPNDLNLVQEQDATEFLNMTPNLSLDLRGNGESSCEDLTSVGPTNKRKHSSAEEGIDCQARGQKFARKAEPKRTKKTKQSGWEVAVATRNGSTASCCTSDDDSNASQESADTGVCPKGKARAARGASTDPQSLYARKRRERINERLKTLQTLVPNGTKVDMSTMLEEAVHYVKFLQLQIKVLSSDDMWMYAPLAYNGMNIGLDLNIYTPERWRTASAAPSTEGREYAGVDRISDLPDGILGDIVSLLPTAEGARTQILKRRWRHIWRCSAPLNLDCCTLVARGGGREAEDELVGLIPSILSSHQGTGRRFHVPSSRHSDRAATIEAWLQSAALDNLQELDLWCTHTYLYDYVPLPPAVFRFSATVRVVTIANCNLRDSAVQGLQFPQLKQLGFKDIIIMEDSLHHMIAACPDLECLMIERSLGFACVRINSLSLRSIGVSTDHPHPHELQFVELVIDNAPCLKRLLHLEMCYHLDMHITVISAPKLETLSCCSSVSRSSTKLSFGSAAIQGLHIDSLTTVVRTVQILAVEMHSLCLDTIIDFMKCFPCLQKLYIKSFVSGNNWWQRKHRNVIKSLDIRLKTIALESYGGNQSDINFVTFFVLNARVLELMTFDVCSEHYTVEFLAEQYRKLQLDKRASRAARFHFTSNRCVRGIPYIGRAELFLPIKCSHVDTSPNLSSFRLSAVFSVCITRNLLRLKKAMWVISLYYSPEFTKQVAVHNPNEMPF

SEQ ID NO: 105 - DNA, 2466 bp, Brachypodium distachyon
atggaggctggagggctgatttctgaggctggctggaccatgtttgacttcccgtcgcaaggcgaggaatcagagatcatgtcgcagctgctaggcgccttcccctcccatcttgaggaaggccatcaggatctgccttggtaccaggcttctgacccatcctactatgactgtaatcttaatacaagtagtgaaagcaatgctagtagtcttgctgttccatccgagtgtatgggctactatttgggtgattcaagtgagtccctggacctgagctcctgcattgcaccaaatgacctgaacttggtccaggagcaagatgcaactgagtttctgaatatgacaccaaatctttcccttgatttacgtgggaatggtgagtcgagctgcgaggatctcacttcggtcggtcctactaacaagcgaaagcactcctcggcagaagaaggaatcgactgccaagcaagaggccagaaattcgccagaaaggctgaaccgaagcgaacaaagaagaccaagcaaagcggatgggaggttgctgttgccaccaggaatggaagcacagcgagctgctgcacctctgatgatgactcaaacgcttctcaagaatctgcagataccggtgtttgtccgaaaggcaaggctcgggctgcccgtggcgcatcaactgatccccagagcctctatgcaaggaaaaggagggaaaggatcaatgagagactgaagacactgcagacccttgtgcccaatggaaccaaagtagatatgagcaccatgcttgaggaggcagtccactacgtgaagttcctgcagcttcagatcaaggtcttgagctctgatgatatgtggatgtatgcgccgctagcatacaacgggatgaacattgggcttgatctgaacatatatactccggagaggtggaggacagcgtccgcggcgccctcaaccgaagggcgtgaatacgccggcgtcgaccgcatcagcgacctccccgacggcatcctcggcgacatcgtctcgttgctccccaccgccgaaggagcccgcacccagatcctcaagcgcaggtggcgccacatctggcgctgctccgcccctctcaacctcgattgctgtaccttggtcgcccgtggcggcggccgtgaggctgaagatgaactcgtcggtctcataccgtccatcctttcttctcaccaaggcaccggccgccgcttccacgtcccctcgtcgcgccactctgaccgagctgctaccattgaagcctggctccaatctgctgccctcgacaatctccaggagctcgatttatggtgcacccacacctatctttacgactatgttccgctgccacccgccgtctttcgcttctccgccaccgtccgtgttgtcaccatcgcaaattgtaacctccgtgacagcgccgtccaaggccttcaattcccacaacttaaacagctcggattcaaagatatcatcatcatggaggattcgctgcaccacatgattgctgcgtgtccagatctcgagtgcttgatgattgaaaggagcttaggttttgcttgcgtccggatcaattcccttagtcttagaagcatcggtgtgagcactgaccaccctcacccacatgagctccagtttgtggaactcgtcattgataatgcaccttgtcttaagagattgctccatcttgaaatgtgttatcaccttgacatgcatataacagtaatctccgcgcctaaactggagaccttgagctgctgttcttctgtgagtcgctcctccaccaaactctcgtttggctccgcggccattcagggattgcacattgatagcctaacaacagtggtgcgcactgtccaaattttagctgtagagatgcattctctttgtctagacacaattattgacttcatgaaatgctttccatgtctgcagaagttgtacattaagtcatttgtaagtggaaacaattggtggcaacgtaaacaccggaacgttatcaaatcccttgacatccgtctcaagacaatagcgttggaaagttatgggggcaatcagtctgacatcaactttgtcacattctttgtcttgaacgcgagagtgctagagttgatgacatttgacgtttgttctgagcattacactgtggagttcttggcagagcaatataggaagcttcagctagataagagggcttcaagagccgctcggttccattttacaagtaaccgatgtgtccgtggtattccgtatatcggacgtgccgagctattcttgcctatcaaatgttctcatgttgacaccagtccaaacttgagtagtttccgtttgtctgcagtattttcagtttgtattacccggaaccttttgcgtttaaaaaaagctatgtgggtcattagtttgtattattctccagaatttacaaaacaagtggccgtgcacaatcccaatgaaatgccgttttag

SEQ ID NO: 106 - protein, 256 aa, Brachypodium distachyon
MEAKCGAIWSSIDARSEDSEMIAHLQSMFWSNSDVALNLCSSNTSGNSCVTASTLPSSLFLPLVDNESYGAAPSVDTGMDSCFDHQHQSITGHKRISHMDEQMKKTRKKSRTVPSVSKALGSSLVDNQMNADIFNQSSSCCSSGEDSIGTSEKSIVANQSDNTSGCKRPSKNMQSLYAKKRRERINEKLRVLQQLIPNGTKVDISTMLEEAVQYVKFLQLQIKVLSSDETWMYAPLAYNGMDIGLTLALRTAANQE

SEQ ID NO: 107 - DNA, 771 bp, Brachypodium distachyon
atggaggccaagtgtggagctatttggagctctatcgatgcgaggagcgaggactctgagatgattgctcacctgcagtccatgttctggagcaacagtgatgttgctctcaacctctgttcgtcaaacaccagtggcaattcttgtgtcacagctagcacattgcctagcagcttgttccttcctcttgtcgataatgagagctatggtgcagcgccatcggtggacaccggcatggattcatgctttgatcaccagcatcagagcattactggtcacaagaggatatcgcacatggatgagcagatgaagaagacgagaaagaagtcccggactgttccatcggtatcaaaggctctgggttccagcctagtcgataatcagatgaatgctgacattttcaatcagagctcctcctgctgcagctcgggagaagattcaattggaacatctgagaaatccattgttgcaaaccagagtgacaatacgagtggttgtaagcggccttcaaagaatatgcaaagcctttatgcaaagaagagaagagagaggatcaacgagaagttgagagtactgcagcagctgattcccaatggcaccaaagttgacatcagcacaatgttggaggaagcagttcagtatgtcaagtttctgcagctgcaaataaaggtcttaagctctgacgagacatggatgtatgcgcccctcgcctacaatggtatggacatcggtctcactctcgctctgagaactgctgcaaaccaagagtga

SEQ ID NO: 108 - protein, 283 aa, Zea mays
MALVREHGGYYGGFDSVEAAAFDTLGYGHGASLGFDASSALFGEGGYAAGGGDAWAGAGASTVLAFNRTTAAAAVGVEEEEEECDAWIDAMDEDDQSSGPAAAAPEARHALTASVGFDASTGCFTLTERASSSSGGAGRPFGLLFPSTSSSGGTPERTAPVRVPQKRTYQAVSPNKKHCGAGRKASKAKLASTAPTKDPQSLAAKQNRRERISERLRALQELVPNGTKVDLVTMLEKAISYVKFLQLQVKVLATDEFWPAQGGKAPEISQVREALDAILSSAS

SEQ ID NO: 109 - DNA, 849 bp, Zea mays
atggcgttggtgagggagcacggtgggtactacggaggcttcgacagcgtcgaggcggcggccttcgacacgctcggctacggccacggcgcgtcgctgggctttgacgcgtcgtcggcgctgttcggggaaggcggttatgcggcgggcggcggggacgcctgggcgggcgcgggggcgtcgaccgtcctggcgttcaaccgcacaacggcagcggcggccgtgggtgtggaagaggaggaggaggagtgcgacgcgtggatcgacgctatggacgaggacgaccagagctccggccccgccgcggcggcgccagaggcgcgccacgcgctgacggcctccgtgggtttcgacgcctccacggggtgcttcaccctgacggagagggcgtcgtcgtcgtcaggcggagcggggcgcccgttcggcctgctgttcccgagcacgtcgtcgtcgggcggcacgcccgagcgcacggcgccggtgcgcgtcccgcagaaacggacctaccaggctgtgagccccaacaagaagcactgcggcgcgggcaggaaggcgagcaaggccaagctcgcgtccacagccccaaccaaagatccccagagcctcgcggccaagcagaaccggcgcgagcggatcagcgagcggctgcgggcgctgcaggagctggtgcccaacggcaccaaggtcgacctggtcaccatgctcgagaaggccatcagctacgttaagttcctccagttgcaagtcaaggttctggcaacagacgaattctggccggcacagggagggaaggcgccggagatctcccaggtgagggaggcgctcgacgccatcttgtcgtcggcgtcg

SEQ ID NO: 110 - protein, 279 aa, Zea mays
MAQFLGAADDHCFTYEYEHVDESMEAIAALFLPTLDTDSANFSSSCFNYAVPPQCWPQPDHSSSVTSLLDPAENFEFPVRDPLPPSGFDPHCAVAYLTEDSSPLHGKRSSVIEEEAANAAPAAKKRKAGAAMQGSKKSRKASKKDNIGDADDDGGYACVDTQSSSSCTSEDGNFEGNTNSSSKKTCARASRGAATEPQSLYARKRRERINERLRILQNLVPNGTKVDISTMLEEAAQYVKFLQLQIKLLSCDDTWMYAPIAYNGINIGNVDLNIYSLQK

SEQ ID NO: 111 - DNA, 840 bp, Zea mays
atggctcagtttcttggggcggctgatgatcactgcttcacctacgagtatgagcatgtggatgagtccatggaagcaatagcagccctgttcttgcctacccttgacaccgactccgccaacttctcctctagctgtttcaactatgctgtccctccacagtgctggcctcagccagaccatagctctagcgttaccagtttgcttgatccagccgagaactttgagtttccagtcagggacccgctccccccaagcggcttcgatccacattgcgctgtcgcctacctcactgaggattcgagccctctgcatggcaaacgttcatcagtcattgaggaagaagcagccaacgccgcacctgctgctaagaagaggaaggctggtgctgcaatgcagggatcaaagaaatccaggaaggcgagcaaaaaggataacatcggcgacgccgacgatgatggcggctatgcctgtgttgacacgcaaagctccagtagctgcacctccgaggacgggaacttcgaaggaaatacgaattcaagctccaagaagacctgcgccagggccagccgcggagcagcaactgaacctcagagtctctatgcaaggaagaggagagagaggatcaacgaaaggttgagaatcttgcagaacttggttccaaatggaacaaaagtagacattagcacgatgctcgaggaagcggcgcagtatgtcaagtttttacagctccagattaagctgttgagctgtgacgacacatggatgtatgcgccaatcgcgtacaatggaattaacatcggcaatgttgatctgaacatctactctctgcaaaagtaa

SEQ ID NO: 112 - protein, 307 aa, Zea mays
  misc_feature 7: Xaa is Ile or Val
MEDGGLXSEAGAWAELGTGGDESEELVAQLLGAFFRSHGEEGRHQLLWSDDQASSDDVHGDGSLAVPLAYDGCCGYLSYSGSNSDELPLGSSSRAAPAGGPPEELLGAAETEYLNNVAAADHPFFKWCGNGEGLDGPTSVVGTLGLGSGRKRARKKSGDEDEDPSTAIASGSGPTSCCTTSDSDSNASPLESADAGARRPKGNENARAAGRGAAAATTTTAEPQSIYARVRRERINERLKVLQSLVPNGTKVDMSTMLEEAVHYVKFLQLQIRVLQLLSSDDTWMYAPIAYNGMGIGIDLRMHGQDR

SEQ ID NO: 113 - DNA, 924 bp, Zea mays
atggaggacggagggttgrtcagcgaggccggcgcctgggccgagctcggcaccggcggcgacgagtcggaggagctggtggcgcagctgctgggcgccttcttccggtcccacggcgaggaaggccggcaccagctgctttggtctgacgaccaagcttcttccgacgacgtgcacggcgacggcagccttgccgtgccgctcgcatacgacggctgctgcggctatctgagctactcaggtagcaactcggacgagctccccctcgggagcagctcccgcgctgcgccagcaggtggcccaccggaggagctgctcggtgcagctgagactgagtacctgaataatgtggccgccgcagaccatcccttcttcaaatggtgtgggaatggtgagggtctggatggtccgacgagcgtcgtgggcacgcttgggcttggctcgggccggaaacgcgcgcgcaagaagagcggggacgaagacgaagacccgagcacggccatcgccagcggaagcggccccacgagctgctgcactacctccgacagcgactcaaacgcgtctcctctggagtccgcggacgccggcgctcgtcgccccaagggcaacgagaatgcccgggcagctggccgcggcgcggcggcggcgacgacgacgacagcggagccccagagcatctacgcaagggtacggagggagcggatcaacgagaggctcaaggtgctgcagagcctggtgcccaacggcaccaaggtggacatgagcaccatgctcgaggaggccgtccactacgtcaagttcctgcagcttcagatcagggtgctgcagctcctgagctccgacgacacgtggatgtacgcgcccatcgcgtacaacgggatgggcatcgggatcgacctccgcatgcatggacaggacagatga

SEQ ID NO: 114 - protein, 141 aa, Zea mays
SKKSRKASKKDCIVDDDDVYVDPQSSGSCTSEEGNFEGNTYSSAKKTCTRASRGGATDPQSLYARKRRERINERLRILQNLVPNGTKVDISTMLEEAAQYVKFLQLQIKLLSSDDMWMYAPIAYNGINISNVDLNIPALQK

SEQ ID NO: 115 - DNA, 414 bp, Zea mays
tcaaagaaatccaggaaggcgagcaaaaaagattgtattgtcgatgacgacgatgtctatgttgacccgcaaagctccggtagctgcacctccgaggaggggaattttgaagggaatacgtattcaagcgcgaaaaagacctgcaccagggccagccgcggaggagcaactgatcctcagagtctctatgcaaggaagaggagagagaggatcaatgaaaggttgagaatcttgcagaacttggtccccaatggaacaaaggttgacattagtacgatgctcgaggaagcagcacagtatgtcaaatttttacagcttcagattaagctgttgagctctgacgacatgtggatgtatgcgccaatcgcgtacaatgggatcaacatcagcaatgttgatctgaacatccctgca

SEQ ID NO: 116 - protein, 65 aa, Carthamus tinctorius
LYHIRRRYPYWVFTDGESTSFARPLLNDSRIRGELLLTLSTTKHCKVTASSMRRSYSMMHDHEKS

SEQ ID NO: 117 - protein, 152 aa, Carthamus tinctorius
KIQRRKSQKLVSKGNESEADHDAVFGQIMKMCGSDNDSNWPRESSTSPRPKEAANLNSNGKTKANRGSATDPQSVYARKRRERINERLRILQSLVPNGTKVDISTMLEDAVQYVKFLQLQIKPLSSDDLWMYAPIAYNGMETGLDSTIPSPR

SEQ ID NO: 118 - protein, 18 aa, Carthamus tinctorius
RLSKVAASFFLKKGKPGA

SEQ ID NO: 119 - DNA, 30 bp, Artificial sequence
  Synthetic sequence Primer
ccaggatccatggcactcgttaatgaccat

SEQ ID NO: 120 - DNA, 31 bp, Artificial sequence
  Synthetic sequence Primer
ccagtcgacttaattggtgatcagattcgaa

SEQ ID NO: 121 - DNA, 29 bp, Artificial sequence
  Synthetic sequence Primer
ccaggatccatgggagaatggagcaacaa

SEQ ID NO: 122 - DNA, 28 bp, Artificial sequence
  Synthetic sequence Primer
ccagtcgactcatctcggtgagctgaga

SEQ ID NO: 123 - DNA, 28 bp, Artificial sequence
  Synthetic sequence Primer
cggggtaccatggaagccatgggagaat

SEQ ID NO: 124 - DNA, 30 bp, Artificial sequence
  Synthetic sequence Primer
cgcggatcctcatctggtcagtgcattgag

SEQ ID NO: 125 - DNA, 30 bp, Artificial sequence
  Synthetic sequence Primer
cggggtaccatggacgtttttgttgatggt

SEQ ID NO: 126 - DNA, 30 bp, Artificial sequence
  Synthetic sequence Primer
cgcggatcctcacataagccgagacaaaag

SEQ ID NO: 127 - protein, 67 aa, Arabidopsis thaliana
RISDDPQTVVARRRRERISEKIRILKRIVPGGAKMDTASMLDEAIRYTKFLKRQVRILQPHSQIGAP

SEQ ID NO: 128 - protein, 67 aa, Physcomitrella patens
RISKDPQSVAARHRRERISDRIRVLQRLVPGGTKMDTASMLDEAIHYVKFLKLQLQVCDTCNLVPVD

SEQ ID NO: 129 - protein, 72 aa, Arabidopsis thaliana
KTKPKPTTSPKDPQSLAAKNRRERISERLKILQELVPNGTKVDLVTMLEKAISYVKFLQVQVKVLATDEFWP

SEQ ID NO: 130 - protein, 72 aa, Arabidopsis thaliana
KIKPKATTSPKDPQSLAAKNRRERISERLKVLQELVPNGTKVDLVTMLEKAIGYVKFLQVQVKVLAADEFWP

SEQ ID NO: 131 - protein, 71 aa, Arabidopsis thaliana
KTRASRGAATDPQSLYARKRRERINERLRILQNLVPNGTKVDISTMLEEAVHYVKFLQLQIKLLSSDDLWM

SEQ ID NO: 132 - protein, 71 aa, Arabidopsis thaliana
KTRASRGAATDPQSLYARKRRERINERLRILQHLVPNGTKVDISTMLEEAVQYVKFLQLQIKLLSSDDLWM

SEQ ID NO: 133 - protein, 71 aa, Arabidopsis thaliana
KTRATKGTATDPQSLYARKRREKINERLKTLQNLVPNGTKVDISTMLEEAVHYVKFLQLQIKLLSSDDLWM

SEQ ID NO: 134 - protein, 71 aa, Arabidopsis thaliana
KAKANRGIASDPQSLYARKRRERINDRLKTLQSLVPNGTKVDISTMLEDAVHYVKFLQLQIKLLSSEDLWM

SEQ ID NO: 135 - protein, 72 aa, Brachypodium distachyon
KSPAPAITSPKDPQSLAAKNRREKISERLRTLQEMVPNGTKVDMVTMLEKAISYVKFLQLQVKVLATDEFWP

SEQ ID NO: 136 - protein, 71 aa, Brachypodium distachyon
KSKAKAAPAKDPQSAVAKVRRERISERLKVLQDLVPNGTKVDMVTMLEKAITYVKFLQLQVKVLATDDFWP

SEQ ID NO: 137 - protein, 74 aa, Brachypodium distachyon
KSSAAATGTSPRKEPQSQAAKSRRERIGERLRALQELVPNGSKVDMVTMLDKAITYVKFMQLQLTVLETDAFWP

SEQ ID NO: 138 - protein, 71 aa, Brachypodium distachyon
KARAARGASTDPQSLYARKRRERINERLKTLQTLVPNGTKVDMSTMLEEAVHYVKFLQLQIKVLSSDDMWM

SEQ ID NO: 139 - protein, 72 aa, Brachypodium distachyon
DNTSGCKRPSKNMQSLYAKKRRERINEKLRVLQQLIPNGTKVDISTMLEEAVQYVKFLQLQIKVLSSDETWM

SEQ ID NO: 140 - protein, 71 aa, Carthamus tinctorius
KTKANRGSATDPQSVYARKRRERINERLRILQSLVPNGTKVDISTMLEDAVQYVKFLQLQIKPLSSDDLWM

SEQ ID NO: 141 - protein, 71 aa, Glycine max
KSRATTGAATDPQSLYARKRRERINERLRILQNLVPNGTKVDISTMLEEAVQYVKFLQLQIKLLSSDDLWM

SEQ ID NO: 142 - protein, 71 aa, Glycine max
KSRATTSAAADPQSLYARKRRERINERLRILQNLVPNGTKVDISTMLEEAVQYVKFLQLQIKLLSSEDLWM

SEQ ID NO: 143 - protein, 71 aa, Lactuca saligna
KRKASRGSATDPQSVYARKRRERINERLRILQNLVPNGTKVDISTMLEEAVEYVKFLQLQIKLLSSDDMWM

SEQ ID NO: 144 - protein, 71 aa, Musa acuminata
KTRSNRWPATDPQSLYAKQRRERINARLRTLQNLVPNGTKVDISTMLEEAVRYVKFLQLQIKLLSSDELWM

SEQ ID NO: 145 - protein, 71 aa, Medicago truncatula
KTRASRGSATDPQSLYARKRRERINERLRVLQNLVPNGTKVDISTMLEEAVNYVKFLQTQIKLLSSDDMWM

SEQ ID NO: 146 - protein, 72 aa, Oryza sativa
SPSPSPSPNKEQPQSAAAKVRRERISERLKVLQDLVPNGTKVDLVTMLEKAINYVKFLQLQVKVLATDEFWP

SEQ ID NO: 147 - protein, 72 aa, Oryza sativa
KAKSAPTTPTKDPQSLAAKNRRERISERLRILQELVPNGTKVDLVTMLEKAISYVKFLQLQVKVLATDEFWP

SEQ ID NO: 148 - protein, 71 aa, Oryza sativa
YSKKQRANNKETQSSAAKSRRERISERLRALQELVPSGGKVDMVTMLDRAISYVKFMQMQLRVLETDAFWP

SEQ ID NO: 149 - protein, 71 aa, Oryza sativa
KARAGRGAATDPQSLYARKRRERINERLKILQNLIPNGTKVDISTMLEEAVHYVKFLQLQIKLLSSDDMWM

SEQ ID NO: 150 - protein, 71 aa, Oryza sativa
KAQSGHRSATDPQSLYARKRRERINERLKILQNLVPNGTKVDISTMLEEAMHYVKFLQLQIKLLSSDEMWM

SEQ ID NO: 151 - protein, 71 aa, Oryza sativa
NSRGHKQCSKDTQSLYAKRRRERINERLRILQQLVPNGTKVDISTMLEEAVQYVKFLQLQIKLLSSDDTWM

SEQ ID NO: 152 - protein, 71 aa, Oryza sativa
KACKHSVSAESSQSYYAKNRRQRINERLRILQELIPNGTKVDISTMLEEAIQYVKFLHLQIKLLSSDEMWM

SEQ ID NO: 153 - protein, 71 aa, Oryza sativa
KSRAGRGAATDPQSLYARKRRERINERLKTLQNLVPNGTKVDISTMLEEAVHYVKFLQLQIKLLSSDEMWM

SEQ ID NO: 154 - protein, 71 aa, Oryza sativa
GTRANRGAATDPQSLYARKRRERINERLRILQNLVPNGTKVDISTMLEEAVQYVKFLQLQIKLLSSDDTWM

SEQ ID NO: 155 - protein, 71 aa, Physcomitrella patens
KPRARQGSANDPQSIAARVRRERISERLKVLQALIPNGDKVDMVTMLEKAISYVQCLEFQIKMLKNDSLWP

SEQ ID NO: 156 - protein, 71 aa, Physcomitrella patens
KPRARQGSANDPQSIAARVRRERISERLKVLQALIPNGDKVDMVTMLEKAITYVQCLELQIKMLKNDSIWP

SEQ ID NO: 157 - protein, 71 aa, Physcomitrella patens
KPRARRGSATDPQSVYARHRREKINERLKTLQHLVPNGAKVDIVTMLDEAIHYVQFLQLQVTLLKSDEYWM

SEQ ID NO: 158 - protein, 71 aa, Physcomitrella patens
RPRVQRGSATDPQSVHARARREKIAERLRKLQHLIPNGGKVDIVTMLDEAVHYVQFLKRQVTLLKSDEYWM

SEQ ID NO: 159 - protein, 71 aa, Physcomitrella patens
KPRAKRGSATDPQSVYARHRREKINERLKSLQNLVPNGAKVDIVTMLDEAIHYVKFLQNQVELLKSDELWI

SEQ ID NO: 160 - protein, 71 aa, Physcomitrella patens
KPRAKRGSATDPQSVYARHRREKINERLKNLQNLVPNGAKVDIVTMLDEAIHYVKFLQTQVELLKSDEFWM

SEQ ID NO: 161 - protein, 71 aa, Physcomitrella patens
RPRAKRGSATDPQSVYARHRREKINERLKTLQRLVPNGEQVDIVTMLEEAIHFVKFLEFQLELLRSDDRWM

SEQ ID NO: 162 - protein, 71 aa, Populus trichocarpa
KPKSAAGPAKDLQSIAAKNRRERISERLKVLQDLVPNGSKVDLVTMLEKAISYVKFLQLQVKVLATDELWP

SEQ ID NO: 163 - protein, 72 aa, Populus trichocarpa
KPKPKSAGPAKDPQSIAAKNRRERISERLKMLQDLVPNGSKVDLVTMLEKAISYVKFLQLQVKVLATDEFWP

SEQ ID NO: 164 - protein, 71 aa, Populus trichocarpa
KTRASRGAATDPQSLYARKRRERINERLRILQTLVPNGTKVDISTMLEEAVQYVKFLQLQIKLLSSEDLWM

SEQ ID NO: 165 - protein, 71 aa, Populus trichocarpa
KTRASKGAATDPQSLYARKRRERINERLRILQNLVPNGTKVDISTMLEEAVQYVKFLQLQIKLLSSEDLWM

SEQ ID NO: 166 - protein, 71 aa, Populus trichocarpa
KTRATRGAATDPQSLYARKRRERINERLKILQNLVPNGTKVDISTMLEEAVHYVNFLQLQIKLLSSDDLWM

SEQ ID NO: 167 - protein, 71 aa, Selaginella moelendorfii
KPRAKQGCANDPQSIAARQRRERISDRLKILQELIPNGSKVDLVTMLEKAINYVKFLQLQVKVLMNDEYWP

SEQ ID NO: 168 - protein, 71 aa, Selaginella moelendorfii; identical to SEQ ID NO: 167

SEQ ID NO: 169 - protein, 71 aa, Selaginella moelendorfii
KPRARQGSANDPQSIAARHRRERISDRLKILQELVPNSTKVDLVTMLEKAINYVKFLQLQVKVLTSDDYWP

SEQ ID NO: 170 - protein, 71 aa, Selaginella moelendorfii; identical to SEQ ID NO: 169

SEQ ID NO: 171 - protein, 71 aa, Selaginella moelendorfii
KPRAKRGSATDPQSIYARQRRERINERLRALQGLVPNGAKVDIVTMLEEAINYVKFLQLQVKLLSSDEYWM

SEQ ID NO: 172 - protein, 71 aa, Selaginella moelendorfii
KPRAKRGSATDPQSVYARHRRERINERLKTLQHLVPNGAKVDIVTMLEEAIHYVKFLQLQVNMLSSDEYWI

SEQ ID NO: 173 - protein, 71 aa, Selaginella moelendorfii
KPRAKRGSATDPQSVYARHRRERINERLKTLQHLVPNGAKVDIVTMLEEAIHYVKFLQLQVNMLSSDEYWT

SEQ ID NO: 174 - protein, 71 aa, Selaginella moelendorfii; identical to SEQ ID NO: 171

SEQ ID NO: 175 - protein, 71 aa, Triticum aestivum
KSKAKVVPTKDPQSAVAKVRRERISERLKVLQDLVPNGTKVDMVTMLEKAITYVKFLQLQVKVLATDEFWP

SEQ ID NO: 176 - protein, 73 aa, Zea mays
KAKLASTAPTKDPQSLAAKQNRRERISERLRALQELVPNGTKVDLVTMLEKAISYVKFLQLQVKVLATDEFWP

SEQ ID NO: 177 - protein, 71 aa, Zea mays
CARASRGAATEPQSLYARKRRERINERLRILQNLVPNGTKVDISTMLEEAAQYVKFLQLQIKLLSCDDTWM

SEQ ID NO: 178 - protein, 81 aa, Zea mays
NARAAGRGAAAATTTTAEPQSIYARVRRERINERLKVLQSLVPNGTKVDMSTMLEEAVHYVKFLQLQIRVLQLLSSDDTWM

SEQ ID NO: 179 - protein, 71 aa, Zea mays
CTRASRGGATDPQSLYARKRRERINERLRILQNLVPNGTKVDISTMLEEAAQYVKFLQLQIKLLSSDDMWM

SEQ ID NO: 180 - protein, 72 aa, Arabidopsis thaliana
KPNRRNVRISDDPQTVVARRRRERISEKIRILKRIVPGGAKMDTASMLDEAIRYTKFLKRQVRILQPHSQIG

SEQ ID NO: 181 - DNA, 25 bp, Artificial sequence
  Synthetic sequence Primer
ggattgatttaattaccatatttat

SEQ ID NO: 182 - DNA, 18 bp, Artificial sequence
  Synthetic sequence Primer
caagtatcaaacgatgtg

SEQ ID NO: 183 - DNA, 25 bp, Artificial sequence
  Synthetic sequence Primer
caccggtaccgaccgttaccgaccg

SEQ ID NO: 184 - DNA, 24 bp, Artificial sequence
  Synthetic sequence Primer
taccggtaccgaaaacgaacggga

SEQ ID NO: 185 - DNA, 23 bp, Artificial sequence
  Synthetic sequence Primer
gttcccaatggcaccaaggtaca

SEQ ID NO: 186 - DNA, 25 bp, Artificial sequence
  Synthetic sequence Primer
cccatttggacgtgaatgtagacac

SEQ ID NO: 187 - DNA, 19 bp, Artificial sequence
  Synthetic sequence Primer
cgtgtggaccgacgtctga

SEQ ID NO: 188 - DNA, 29 bp, Artificial sequence
  Synthetic sequence Primer
cattttataataacgctgcggacatctac

SEQ ID NO: 189 - DNA, 58 bp, Artificial sequence
  Synthetic sequence Primer
gggacaactttgtatagaaaagttgttctcaaagagggacaagaccaaagcccatgac

SEQ ID NO: 190 - DNA, 61 bp, Artificial sequence
  Synthetic sequence Primer
ggggactgcttttttgtacaaacttgctagacactaataagtttgataagtgattttttgt

SEQ ID NO: 191 - DNA, 59 bp, Artificial sequence
  Synthetic sequence Primer
ggggacagctttcttgtacaaagtggccatggcactcgttaatgaccatcccaacgaga

SEQ ID NO: 192 - DNA, 57 bp, Artificial sequence
  Synthetic sequence Primer
ggggacaactttgtataataaagttgctgataaatcgagatcttaggtatgtcgtcc

SEQ ID NO: 193 - DNA, 53 bp, Artificial sequence
  Synthetic sequence Primer
ggggacaactttgtatagaaaagttgtgataatggattggagaaaaattaaag

SEQ ID NO: 194 - DNA, 52 bp, Artificial sequence
  Synthetic sequence Primer
ggggactgcttttttgtacaaacttgtattgcaatgttcgttaatgagtgac

SEQ ID NO: 195 - DNA, 53 bp, Artificial sequence
  Synthetic sequence Primer
ggggacagctttcttgtacaaagtgggtaattacatctcaaccccaaattctt

SEQ ID NO: 196 - DNA, 49 bp, Artificial sequence
  Synthetic sequence Primer
acaactttgtataataaagttgatgtataatttccgaagatgcttaaaa

SEQ ID NO: 197 - DNA, 27 bp, Artificial sequence
  Synthetic sequence Primer
ccaggatccatggcaggtccagcagga

SEQ ID NO: 198 - DNA, 30 bp, Artificial sequence
  Synthetic sequence Primer
ccagtcgacttagtcagcagaaggctgatt

SEQ ID NO: 199 - DNA, 30 bp, Artificial sequence
  Synthetic sequence Primer
cctctagaagtacttgtgatccacagccta

SEQ ID NO: 200 - DNA, 25 bp, Artificial sequence
  Synthetic sequence Primer
ggctcgagccgtactgggtggtttg

SEQ ID NO: 201 - DNA, 33 bp, Artificial sequence
  Synthetic sequence Primer
ccgttaacttctacatgttgcgttatttatggt

SEQ ID NO: 202 - DNA, 43 bp, Artificial sequence
  Synthetic sequence Primer
ccggcgcgccaatatttatataaataagcataatacacttcga

SEQ ID NO: 203 - DNA, 29 bp, Artificial sequence
  Synthetic sequence Primer
gcaacgcgtgggtttgatcaaagacggaa

SEQ ID NO: 204 - DNA, 29 bp, Artificial sequence
  Synthetic sequence Primer
gctactagtcgtcaacctaacccaaacat

SEQ ID NO: 205 - DNA, 29 bp, Artificial sequence
  Synthetic sequence Primer
gagggatccgtgaggtgaaagcagtgaaa

SEQ ID NO: 206 - DNA, 28 bp, Artificial sequence
  Synthetic sequence Primer
ccaaagcttaggcctgtgaactcggaca

SEQ ID NO: 207 - DNA, 26 bp, Artificial sequence
  Synthetic sequence Primer
gctgctagggtaacataaacattctt

SEQ ID NO: 208 - DNA, 21 bp, Artificial sequence
  Synthetic sequence Primer
ctggacactggaatgaaccta

SEQ ID NO: 209 - DNA, 20 bp, Artificial sequence
  Synthetic sequence Primer
cccatggagtcaaagattca

SEQ ID NO: 210 - DNA, 19 bp, Artificial sequence
  Synthetic sequence Primer
ccgcgaattcgagctcggt

SEQ ID NO: 211 - DNA, 24 bp, Artificial sequence
  Synthetic sequence Primer
cccaaatatgcatttttaatcttt

SEQ ID NO: 212 - DNA, 22 bp, Artificial sequence
  Synthetic sequence Primer
gcgacaatccagcagcctctat

SEQ ID NO: 213 - DNA, 20 bp, Artificial sequence
  Synthetic sequence Primer
gtaggagggcgtggatatgt

SEQ ID NO: 214 - DNA, 15 bp, Artificial sequence
  Synthetic sequence Primer
cgagtgctggggcgt

SEQ ID NO: 215 - DNA, 20 bp, Artificial sequence
  Synthetic sequence Primer
gggacctcaaggatgcagca

SEQ ID NO: 216 - DNA, 20 bp, Artificial sequence
  Synthetic sequence Primer
cgaactcaataacgtcagga

SEQ ID NO: 217 - DNA, 22 bp, Artificial sequence
  Synthetic sequence Primer
tcccagaatcgctaagattgcc

SEQ ID NO: 218 - DNA, 18 bp, Artificial sequence
  Synthetic sequence Primer
cctttcccttaagctctg

SEQ ID NO: 219 - DNA, 18 bp, Artificial sequence
  Synthetic sequence Primer
ctcacacgggagagagca

SEQ ID NO: 220 - DNA, 19 bp, Artificial sequence
  Synthetic sequence Primer
cttcgattcttggctgcta

SEQ ID NO: 221 - DNA, 21 bp, Artificial sequence
  Synthetic sequence Primer
gcctagcagccaagaaccgaa

SEQ ID NO: 222 - DNA, 22 bp, Artificial sequence
  Synthetic sequence Primer
ctcatcggctgcaagtacctta

SEQ ID NO: 223 - DNA, 24 bp, Artificial sequence
  Synthetic sequence Primer
ctggttggttaggagatcttgcat

SEQ ID NO: 224 - DNA, 24 bp, Artificial sequence
  Synthetic sequence Primer
gttgtaatttggtccatttctgct

SEQ ID NO: 225 - DNA, 20 bp, Artificial sequence
  Synthetic sequence Primer
gggacctcaaggatgcagca

SEQ ID NO: 226 - DNA, 20 bp, Artificial sequence
  Synthetic sequence Primer
cgaactcaataacgtcagga

SEQ ID NO: 227 - DNA, 26 bp, Artificial sequence
  Synthetic sequence Primer
gagataggagcatctgtaccgcttgt

SEQ ID NO: 228 - DNA, 18 bp, Artificial sequence
  Synthetic sequence Primer
cgcatggtgggatcggct